(19) United States
(12) Patent Application Publication
(10) Pub. No.: US 2021/0010692 A1
(43) Pub. Date: Jan. 14, 2021

(54) MEDICAL RESIDENTIAL AND LABORATORY USES OF PURIFIED AIR

(71) Applicant: Lifeaire Systems, LLC, Allentown, PA (US)

(72) Inventor: Kathryn C. WORRILOW, Fogelsville, PA (US)

(21) Appl. No.: 16/769,336

(22) PCT Filed: Apr. 2, 2019
(86) PCT No.: PCT/US2019/025292

§ 371 Date: Jun. 3, 2020

Related U.S. Application Data
(60) Provisional application No. 62/651,316, filed on Apr. 2, 2018.

(51) Int. Cl. F24F 3/16; A61L 9/20; B01D 46/00

(52) U.S. Cl. CPC F24F 3/16; A61L 9/20; A61L 2209/16; A61L 2209/14; B01D 46/0028

(57) ABSTRACT

The air purification technology and methods of the disclosure will reduce the levels of airborne biological, chemical, and particulate contamination and thus improve outcomes in, for example, all areas of basic, applied, commercial, industrial, biological, and chemical research and manufacturing, as well as hospital settings, and long-term care facilities, as set forth herein.

This application claims benefit of U.S. Ser. No. 62/651,316 filed Apr. 2, 2018, the entirety of which is incorporated herein by reference.

### BACKGROUND OF THE INVENTION

The air purification technology and methods of the disclosure will reduce the levels of airborne biological, chemical, and particulate contamination and thus improve outcomes in all areas of, for example, basic, applied, commercial, industrial, biological, and chemical research and manufacturing, as well as hospital settings, and long-term care facilities, as set forth herein.

The system and methods of the disclosure were initially designed to protect the environment of the human embryo in the In Vitro Fertilization (IVF) laboratory. Because it was designed to protect the human embryo, it was critical that the system remediate all chemical and biological contaminants from the air. It has been designed to kill the anthrax spore, the most difficult biological to kill. The system has been tested by the National Homeland Security Research Center and patents have recently been issued by the U.S. and International Patent Offices. Because of its effectiveness towards the anthrax spore, the system and methods of the disclosure are able to remediate airborne pathogens such as Clostridium difficile, MRSA, aspergillus, streptococcus, etc., each representing a significant source and causality of hospital acquired infections (HAIs) and a consistent threat to both the residential and clinical environments. The System has been in place for over 4 years, protecting and supporting the growth of the human embryo, the most sensitive cell in physiology. By removing a significant source of airborne pathogens from the environment, the technology is associated with significant increase in clinical outcomes and improved patient care. The system has been tested by third party analysis, and while effective in removing all chemical and biological pathogens from the air, it produces no byproducts.

The goal is a transformational air purification technology (in-duct or in-room) towards the removal of infectious pathogens and in the concomitant reduction of resident illness, HAI rates and increased cost avoidance.

The system and methods of the disclosure remove chemical and biological pathogens from the air and produces no byproducts. The air purification technology has been in place for 4 years in the protection of the human embryo. As a result of its effectiveness in removing airborne chemical and biological pathogens, physicians have realized a significant increase in their clinical outcomes and level of patient care offered.

It has been estimated that 1.7 million or 1 of every 20 inpatients within the United States develops an HAI each year. This number represents 5-10% of all patients admitted annually. Approximately 99,000 of patients afflicted with an HAI die each year. The number of estimated patients with an HAI exceeds that of any required reportable disease in the U.S. and the number of deaths attributed to HAIs exceeds many of the top ten leading causes of death reported from the U.S. vital statistics. It is understood that these estimates are likely under-representing the true caseload due to erroneous reporting and the fact that much of the data are voluntarily reported. As of 2014, only 30 states and the District of Columbia support mandatory reporting criteria. Apart from the morbidity and mortality associated with HAIs, it has been estimated that HAIs cost the U.S. between $35B and $88B annually.

Additionally, more than 1.5 million residents in United States reside in long-term care facilities (LTCF). In recent years, the acuity of illness of LTCF residents has increased. The risk of LTCF residents developing HAIs approaches the levels seen in acute care hospital patients, impacting resident wellness, quality of life, and overall costs. Historically, it has been understood that the source of the pathogens responsible for HAIs and general illnesses (influenza, pneumonia, tuberculosis, etc.) are associated with the patient and surface areas. Infection control protocols, patient preparation and hand washing protocols have been implemented and have been helpful in reducing overall infection rates. Recent literature from the CDC and leading scientists has demonstrated that significant sources of pathogens responsible for HAIs are airborne. Specifically, the data indicates that of those pathogens responsible for HAIs, 69-80% are airborne. Infectious airborne pathogens include but are not limited to Clostridium difficile, antibiotic resistant MRSA, aspergillus, pseudomonas, streptococcus, staphylococcus, tuberculosis, smallpox and influenza. Many of the infectious pathogens can remain airborne and viable for weeks to months at a time. Physical filtration such as high efficiency particulate air (HEPA) filtration is commonly used to reduce levels of airborne pathogens. Physical filtration captures only those infectious pathogens of a certain size. Many bacterial and viral pathogens responsible for infections and illness are not captured. Those pathogens captured often continue to proliferate on the filter and thus remain a permanent threat to the patient, resident and staff in the space being protected. The proprietary technology offered by the System and methods of the disclosure does not capture the pathogens. It removes all airborne bacterial and viral pathogens by destroying the DNA and RNA associated with the viable particulates. The technology renders the pathogens noninfectious on a single pass through the system while not disturbing patient, resident or personnel operations.

The system and methods of the disclosure provide sterile air to the life sciences and clinical environment by killing/removing airborne biological, particulate, and chemical pathogens from all source/outside and recirculated air from the space, thereby removing the variable of air from all ongoing processes, leading to, for example, improved living cell culture, gene therapy and viral production processes.

The system and methods of the disclosure employ reformulated and optimized Ultraviolet Germicidal Irradiation (UVGI) technology such that infectious biologicals are inactivated on a single pass. The system and methods of the disclosure also employ proprietary and patented technologies that include multiple levels of filtration, purification and inactivation media such that chemical, particulate and infectious biological pathogens are removed from the outside and recirculated air. The system design was mathematically and genomically modeled to kill and inactivate Clostridium difficile, MRSA, aspergillus, pseudomonas, streptococcus, staphylococcus, tuberculosis, smallpox, influenza and other such infectious biologicals on a single pass through the system. The revolutionary technology offered by the system and methods of the disclosure has the potential to dramatically reduce contaminants within the active clinical setting and concomitantly impact the associated illness and HAI rate.

The air purification technology and methods of the disclosure will reduce the levels of airborne biological, chemical, and particulate contamination and thus improve outcomes in all areas of, for example, basic, applied, commercial, industrial, biological, and chemical research and manufacturing, as well as hospital settings, and LTCF, as set forth herein. The air purification technology offered by the system and methods of the disclosure will reduce the levels of airborne biological, chemical, and particulate contamination and thus impact the illness, HAI, length of stay (LOS) and readmission rate in the long-term care clinical environment.

Existing filtration devices have been found insufficient to optimize air quality to truly acceptable levels for example, basic, applied, commercial, industrial, biological, and chemical research and manufacturing, as well as hospital settings, and long-term care facilities. For example, it has been found that laboratory air that had been filtered with only HEPA filters was actually of lesser quality than outside air. Additionally, some filters produce by-products or other contaminants that actually detract from the quality of the air in for example, basic, applied, commercial, industrial, biological, and chemical research and manufacturing, as well as hospital settings, and LTCF. For example, carbon filters can create carbon dusting that is harmful to the IVF process. This is not to say, however, that carbon filters or HEPA filters should not be used to treat air supplied to, for example, basic, applied, commercial, industrial, biological, and chemical research and manufacturing, as well as hospital settings, and LTCF. On the contrary, it is preferred that carbon filters, HEPA filters, or their respective equivalents, are included among filtration media used to treat air supplied to an for example, basic, applied, commercial, industrial, biological, and chemical research and manufacturing, as well as hospital settings, and LTCF. Attaining optimal air quality in for example, basic, applied, commercial, industrial, biological, and chemical research and manufacturing, as well as hospital settings, and LTCF or other substantially enclosed space requires proper selection, combination and sequencing of various filtration media. Other embodiments of air purification devices and methods may be found in U.S. Pat. Nos. 9,522,210; 8,252,100; 8,252,099; U.S. Patent Application Publication No. 2016/0199769; U.S. Patent Application Publication No. 2015/0367017; U.S. Patent Application Publication No. 2012/0283508; U.S. Patent Application Publication No. 2012/0014856; and U.S. Patent Application Publication No. 2011/0232481, all which are incorporated herein by reference in their entireties.

All references cited herein are incorporated herein by reference in their entireties.

### BRIEF SUMMARY OF THE INVENTION

The disclosure provides a method of reducing airborne biological contaminants, chemical contaminants, and particulate contaminants in a health care facility by providing purified air, the method comprising the steps of: providing an air purifier in a health care facility; providing source air which is to be purified to the air purifier; purifying the source air with the air purifier, thereby providing purified air, wherein the purified air has characteristics selected from the group consisting of: a. a total volatile organic compound (TVOC) content of less than about 5 ppb; b. a Biologicals content of less than about 1 CFU/M3; c. a Particulate content of from about 1,000 0.3 micrometer particles per ft3 of air to about 30,000 0.3 micrometer particles per ft3 of air, or from about 600 0.5 micrometer particles per ft3 of air to about 10,000 0.5 micrometer particles per ft3 of air; and d. combinations thereof, supplying the purified air to the health care facility. The disclosure provides a method wherein the health care facility is a hospital. The disclosure provides a method wherein the health care facility is selected from the group consisting of a medical unit, a surgical unit, a critical care unit, an intensive care unit, an emergency care unit, a pediatric unit, an emergency unit, an outpatient unit, a specialty care unit, a dermatology unit, an endocrinology unit, a gastroenterology, an internal medicine unit, an oncology unit, a neurology unit, an orthopedic unit, an ophthalmic unit, an ear nose and throat unit, a neonatal unit, an obstetrics and gynecology unit, a cardiac unit, a psychiatric unit, a post-operative recovery unit, a radiology unit, a plastic surgery unit, an urology unit, and combinations thereof. The disclosure provides a method wherein the purified air has reduced biological contaminants compared to non-purified or source air, and wherein the biological contaminants are selected from the group consisting of viral contaminants, bacterial contaminants, fungal contaminants, parasitic contaminants, and combinations thereof. The disclosure provides a method wherein the purified air has reduced viral contaminants compared to non-purified or source air, and wherein the viral contaminant is selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, Astrovirus, HAV, Rotavirus, and combinations thereof. The disclosure provides a method wherein the purified air has reduced bacterial contaminants compared to non-purified air, and wherein the bacterial contaminant is selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof. The disclosure provides a method wherein the purified air has reduced fungal contaminants compared to non-purified air, and wherein the fungal contaminant is selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof. The disclosure provides a method wherein the purified air has reduced parasitic contaminants compared to non-purified air, and wherein the parasitic contaminant is selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof. The disclosure provides a method wherein the purified air has reduced chemical contaminants compared to non-purified air, and wherein the chemical contaminants are selected from the group consisting of tobacco smoke, engine exhaust, volatile organic compounds (VOCs) from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, the like, and combinations thereof. VOCs include organic solvents, certain paint additives, aerosol spray can propellants, fuels (such as gasoline and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone. The disclosure provides a method wherein the purified air has reduced particulate contaminants compared to non-purified air, and wherein the particulate contaminants are selected from the group consisting of indoor allergens, dust mite feces, dander, cockroach-derived allergens, and combinations thereof. The disclosure provides a method wherein the air purifier is selected from the group consisting of a unit installed into an existing HVAC system or air handling unit (AHU), an air purifier which is a stand-alone unit that is not part of an HVAC system or AHU, an air purifier which is a stand-alone unit which purifies in-room air, an air purifier which is an in-room unit that purifies in-room air, an air purifier which is an in-room unit that is an in-ceiling unit that purifies in-room air, an air purifier which is an in-room unit that is a ceiling unit that purifies in-room air, and combinations thereof.

The disclosure provides a method of reducing HAIs in a health care facility by providing purified air, the method comprising the steps of: providing an air purifier in a health care facility; providing source air which is to be purified to the air purifier; purifying the source air with the air purifier, thereby providing purified air, wherein the purified air has characteristics selected from the group consisting of: a. a TVOC content of less than about 5 ppb; b. a Biologicals content of less than about 1 CFU/M3; c. a Particulate content of from about 1,000 0.3 micrometer particles per ft3 of air to about 30,000 0.3 micrometer particles per ft3 of air, or from about 600 0.5 micrometer particles per ft3 of air to about 10,000 0.5 micrometer particles per ft3 of air; and d. combinations thereof, supplying the purified air to the health care facility. The disclosure provides a method wherein the health care facility is a hospital. The disclosure provides a method wherein the health care facility is selected from the group consisting of a medical unit, a surgical unit, a critical care unit, an intensive care unit, an emergency care unit, a pediatric unit, an emergency unit, an outpatient unit, a specialty care unit, a dermatology unit, an endocrinology unit, a gastroenterology, an internal medicine unit, an oncology unit, a neurology unit, an orthopedic unit, an ophthalmic unit, an ear nose and throat unit, a neonatal unit, an obstetrics and gynecology unit, a cardiac unit, a psychiatric unit, a post-operative recovery unit, a radiology unit, a plastic surgery unit, an urology unit, and combinations thereof. The disclosure provides a method wherein the purified air has reduced biological contaminants compared to non-purified or source air, and wherein the biological contaminants are selected from the group consisting of viral contaminants, bacterial contaminants, fungal contaminants, parasitic contaminants, and combinations thereof. The disclosure provides a method wherein the purified air has reduced viral contaminants compared to non-purified or source air, and wherein the viral contaminant is selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, Astrovirus, HAV, Rotavirus, and combinations thereof. The disclosure provides a method wherein the purified air has reduced bacterial contaminants compared to non-purified air, and wherein the bacterial contaminant is selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof. The disclosure provides a method wherein the purified air has reduced fungal contaminants compared to non-purified air, and wherein the fungal contaminant is selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof. The disclosure provides a method wherein the purified air has reduced parasitic contaminants compared to non-purified air, and wherein the parasitic contaminant is selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof. The disclosure provides a method wherein the purified air has reduced chemical contaminants compared to non-purified air, and wherein the chemical contaminants are selected from the group consisting of tobacco smoke, engine exhaust, volatile organic compounds from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, the like, and combinations thereof. VOCs include organic solvents, certain paint additives, aerosol spray can propellants, fuels (such as gasoline and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone. The disclosure provides a method wherein the purified air has reduced particulate contaminants compared to non-purified air, and wherein the particulate contaminants are selected from the group consisting of indoor allergens, dust mite feces, dander, cockroach-derived allergens, and combinations thereof. The disclosure provides a method wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier which is an in-room unit that is an in-ceiling unit that purifies in-room air, an air purifier which is an in-room unit that is a ceiling unit that purifies in-room air, and combinations thereof.

The disclosure provides a method of reducing healthcare associated infections (HAIs) in a LTCF by providing purified air, the method comprising the steps of: providing an air purifier in a health care facility; providing source air which is to be purified to the air purifier; purifying the source air with the air purifier, thereby providing purified air, wherein the purified air has characteristics selected from the group consisting of: a. a TVOC content of less than about 5 ppb; b. a Biologicals content of less than about 1 CFU/M3; c. a Particulate content of from about 1,000 0.3 micrometer particles per ft3 of air to about 30,000 0.3 micrometer particles per ft3 of air, or from about 600 0.5 micrometer particles per ft3 of air to about 10,000 0.5 micrometer particles per ft3 of air; and d. combinations thereof, supplying the purified air to the LTCF. The disclosure provides a method wherein the LTCF is selected from the group consisting of an Inpatient Hospice, Long Term Care Unit, Long Term Care Alzheimer's Unit, Long Term Care Behavioral Health/Psych Unit, Ventilator Dependent Unit, a Long Term Care Rehabilitation Unit, and combinations thereof. The disclosure provides a method wherein purified air has reduced biological contaminants compared to non-purified air, and wherein the biological contaminants are selected from the group consisting of viral contaminants, bacterial contaminants, fungal contaminants, parasitic contaminants, and combinations thereof. The disclosure provides a method wherein the purified air has reduced viral contaminants compared to non-purified air, and wherein the viral contaminant is selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, Astrovirus, HAV, Rotavirus, and combinations thereof. The disclosure provides a method wherein the purified air has reduced bacterial contaminants compared to non-purified air, and wherein the bacterial contaminant is selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof. The disclosure provides a method wherein the purified air has reduced fungal contaminants compared to non-purified air, and wherein the fungal contaminant is selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum; and combinations thereof. The disclosure provides a method wherein the purified air has reduced parasitic contaminants compared to non-purified air, and wherein the parasitic contaminant is selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium; and combinations thereof. The disclosure provides a method wherein the purified air has reduced chemical contaminants compared to non-purified air, and wherein the chemical contaminants are selected from the group consisting of tobacco smoke, engine exhaust, VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, the like, and combinations thereof. VOCs include organic solvents, certain paint additives, aerosol spray can propellants, fuels (such as gasoline and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone. The disclosure provides a method wherein the purified air has reduced particulate contaminants compared to non-purified air, and wherein the particulate contaminants are selected from the group consisting of indoor allergens, dust mite feces, dander, cockroach-derived allergens, and combinations thereof. The disclosure provides a method wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier is an in-room unit that is an in-ceiling unit that purifies in-room air, an air purifier which is an in-room unit that is a ceiling unit that purifies in-room air, and combinations thereof.

The disclosure provides a method of reducing airborne biological contaminants, chemical contaminants, and particulate contaminants in a LTCF by providing purified air, the method comprising the steps of: providing an air purifier in a LTCF; providing source air which is to be purified to the air purifier; purifying the source air with the air purifier, thereby providing purified air, wherein the purified air has characteristics selected from the group consisting of: a. a TVOC content of less than about 5 ppb; b. a Biologicals content of less than about 1 CFU/M3; c. a Particulate content of from about 1,000 0.3 micrometer particles per ft3 of air to about 30,000 0.3 micrometer particles per ft3 of air, or from about 600 0.5 micrometer particles per ft3 of air to about 10,000 0.5 micrometer particles per ft3 of air; and d. combinations thereof, supplying the purified air to the long term care facility. The disclosure provides a method wherein the LTCF is selected from the group consisting of an Inpatient Hospice, Long Term Care Unit, Long Term Care Alzheimer's Unit, Long Term Care Behavioral Health/Psych Unit, Ventilator Dependent Unit, a Long Term Care Rehabilitation Unit, and combinations thereof. The disclosure provides a method wherein the purified air has reduced biological contaminants compared to non-purified or source air, and wherein the biological contaminants are selected from the group consisting of viral contaminants, bacterial contaminants, fungal contaminants, parasitic contaminants, and combinations thereof. The disclosure provides a method wherein the purified air has reduced viral contaminants compared to non-purified air, and wherein the viral contaminant is selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, Astrovirus, HAV, Rotavirus, and combinations thereof. The disclosure provides a method wherein the purified air has reduced bacterial contaminants compared to non-purified air, and wherein the bacterial contaminant is selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof. The disclosure provides a method wherein the purified air has reduced fungal contaminants compared to non-purified air, and wherein the fungal contaminant is selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum; and combinations thereof. The disclosure provides a method wherein the purified air has reduced parasitic contaminants compared to non-purified air, and wherein the parasitic contaminant is selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium; and combinations thereof. The disclosure provides a method wherein the purified air has reduced chemical contaminants compared to non-purified air, and wherein the chemical contaminants are selected from the group consisting of tobacco smoke, engine exhaust, volatile organic compounds from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, the like, and combinations thereof. VOCs include organic solvents, certain paint additives, aerosol spray can propellants, fuels (such as gasoline and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone. The disclosure provides a method wherein the purified air has reduced particulate contaminants compared to non-purified air, and wherein the particulate contaminants are selected from the group consisting of indoor allergens, dust mite feces, dander, and cockroach-derived allergens. The disclosure provides a method wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier is an in-room unit that is an in-ceiling unit that purifies in-room air, an air purifier which is an in-room unit that is a ceiling unit that purifies in-room air, and combinations thereof.

The disclosure provides a method of reducing airborne biological contaminants, chemical contaminants, and particulate contaminants in a laboratory facility by providing purified air, the method comprising the steps of: providing an air purifier in a laboratory facility; providing source air which is to be purified to the air purifier; purifying the source air with the air purifier, thereby providing purified air, wherein the purified air has characteristics selected from the group consisting of: a. a TVOC content of less than about 5 ppb; b. a Biologicals content of less than about 1 CFU/M3; c. a Particulate content of from about 1,000 0.3 micrometer particles per ft3 of air to about 30,000 0.3 micrometer particles per ft3 of air, or from about 600 0.5 micrometer particles per ft3 of air to about 10,000 0.5 micrometer particles per ft3 of air; and d. combinations thereof, supplying the purified air to the laboratory facility. The disclosure provides a method wherein the laboratory facility is selected from the group consisting of a biochemistry, bioinformatics, biotechnology, cell biology, chemical biology, cell therapy, cell and organ transplantation, developmental biology, ecology, endocrinology, epidemiology, evolution, genetics, gene therapy, genomics, gerontology, immunology, infectious diseases, microbiology, molecular biology, nephrology, neurology, ophthalmology, pediatrics, pharmacology, physiology, plant biology, psychiatry, public health, structural biology, surgery, urology, drug discovery, molecular therapeutics, epidemiology, carcinogenesis, inflammation, pain, nutrition, reproduction, virology, toxicology, pathology, dermatology, gastroenterology, musculoskeletal studies, pregnancy, pulmonary studies, breast cancer, cardiovascular studies, cerebrospinal research, allergy and asthma studies, hepatology, atherosclerosis, diabetes studies, hematology, oncology, osteoporosis studies, rheumatology studies, vaccine studies, circadian rhythms studies, proteome studies, respiratory research, thrombosis studies, anti-viral and anti-microbial and anti-parasite studies gene regulation studies, cell culture studies of all kinds, organ culture and transplant studies of all kinds, protein production studies, in vitro and in vivo cell and organ growth and differentiation studies of all kinds, and combinations thereof. The disclosure provides a method wherein the purified air has reduced biological contaminants compared to non-purified or source air, and wherein the biological contaminants are selected from the group consisting of viral contaminants, bacterial contaminants, fungal contaminants, parasitic contaminants, and combinations thereof. The disclosure provides a method wherein the purified air has reduced viral contaminants compared to non-purified air, and wherein the viral contaminant is selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, Astrovirus, HAV, Rotavirus, and combinations thereof. The disclosure provides a method wherein the purified air has reduced bacterial contaminants compared to non-purified air, and wherein the bacterial contaminant is selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof. The disclosure provides a method wherein the purified air has reduced fungal contaminants compared to non-purified air, and wherein the fungal contaminant is selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum; and combinations thereof. The disclosure provides a method wherein the purified air has reduced parasitic contaminants compared to non-purified air, and wherein the parasitic contaminant is selected from the group consisting or Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium; and combinations thereof. The disclosure provides a method wherein the purified air has reduced chemical contaminants compared to non-purified air, and wherein the chemical contaminants are selected from the group of tobacco smoke, engine exhaust, volatile organic compounds from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, the like, and combinations thereof. VOCs include organic solvents, certain paint additives, aerosol spray can propellants, fuels (such as gasoline, and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone. The disclosure provides a method wherein the purified air has reduced particulate contaminants compared to non-purified air, and wherein the particulate contaminants are selected from the group consisting of indoor allergens, dust mite feces, dander, cockroach-derived allergens, and combinations thereof. The disclosure provides a method wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier which is an in-room unit that is an in-ceiling unit that purifies in-room air, an air purifier which is an in-room unit that is a ceiling unit that purifies in-room air, and combinations thereof.

The disclosure provides a method of providing purified air to a laboratory, the method comprising the steps of: providing an air purifier in a laboratory facility; providing source air which is to be purified to the air purifier; purifying the source air with the air purifier, thereby providing purified air, wherein the purified air has characteristics selected from the group consisting of: a. a TVOC content of less than about 5 ppb; b. a Biologicals content of less than about 1 CFU/M3; c. a Particulate content of from about 1,000 0.3 micrometer particles per ft3 of air to about 30,000 0.3 micrometer particles per ft3 of air, or from about 600 0.5 micrometer particles per ft3 of air to about 10,000 0.5 micrometer particles per ft3 of air; and d. combinations thereof, supplying the purified air to the laboratory facility. The disclosure provides a method wherein the laboratory facility is selected from the group consisting of a biochemistry, bioinformatics, biotechnology, cell biology, chemical biology, cell therapy, cell and organ transplantation, developmental biology, ecology, endocrinology, epidemiology, evolution, genetics, gene therapy, genomics, gerontology, immunology, infectious diseases, microbiology, molecular biology, nephrology, neurology, ophthalmology, pediatrics, pharmacology, physiology, plant biology, psychiatry, public health, structural biology, surgery, urology, drug discovery, molecular therapeutics, epidemiology, carcinogenesis, inflammation, pain, nutrition, reproduction, virology, toxicology, pathology, dermatology, gastroenterology, musculoskeletal studies, pregnancy, pulmonary studies, breast cancer, cardiovascular studies, cerebrospinal research, allergy and asthma studies, hepatology, atherosclerosis, diabetes studies, hematology, oncology, osteoporosis studies, rheumatology studies, vaccine studies, circadian rhythms studies, proteome studies, respiratory research, thrombosis studies, anti-viral and anti-microbial and anti-parasite studies gene regulation studies, cell culture studies of all kinds, organ culture and transplant studies of all kinds, protein production studies, in vitro and in vivo cell and organ growth and differentiation studies of all kinds, and combinations thereof. The disclosure provides a method wherein the purified air has reduced biological contaminants compared to non-purified air, wherein the biological contaminants are selected from the group consisting of viral contaminants, bacterial contaminants, fungal contaminants, parasitic contaminants, and combinations thereof. The disclosure provides a method wherein the purified air has reduced viral contaminants compared to non-purified air, wherein the viral contaminants are selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, Astrovirus, HAV, Rotavirus, and combinations thereof. The disclosure provides a method wherein the purified air has reduced bacterial contaminants compared to non-purified air, wherein the bacterial contaminants are selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof. The disclosure provides a method wherein the purified air has reduced fungal contaminants compared to non-purified air, wherein the fungal contaminants are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum; and combinations thereof. The disclosure provides a method wherein the purified air has reduced parasitic contaminants compared to non-purified air, wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium; and combinations thereof. The disclosure provides a method wherein the purified air has reduced chemical contaminants compared to non-purified air, wherein the chemical contaminants are selected from the group consisting of tobacco smoke, engine exhaust, volatile organic compounds from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, the like, and combinations thereof. VOCs include organic solvents, certain paint additives, aerosol spray can propellants, fuels (such as gasoline, and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone. The disclosure provides a method wherein the purified air has reduced particulate contaminants compared to non-purified air, wherein the particulate contaminants are selected from the group consisting of indoor allergens, dust mite feces, dander, cockroach-derived allergens, and combinations thereof. The disclosure provides a method wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier which is an in-room unit that is an in-ceiling unit that purifies in-room air, an air purifier which is an in-room unit that is a ceiling unit that purifies in-room air, and combinations thereof.

The disclosure provides a method of performing a laboratory process in purified air, the method comprising the steps of: providing an air purifier in a laboratory process facility; providing source air which is to be purified to the air purifier; purifying the source air with the air purifier, thereby providing purified air, wherein the purified air has characteristics selected from the group consisting of: a. a TVOC content of less than about 5 ppb; b. a Biologicals content of less than about 1 CFU/M3; c. a Particulate content of from about 1,000 0.3 micrometer particles per ft3 of air to about 30,000 0.3 micrometer particles per ft3 of air, or from about 600 0.5 micrometer particles per ft3 of air to about 10,000 0.5 micrometer particles per ft3 of air; and d. combinations thereof, supplying the purified air to the laboratory facility, and performing the laboratory process. The disclosure provides a method wherein the laboratory process is selected from the group consisting of cell culture, gene therapy, viral production, biochemistry, bioinformatics, biotechnology, cell biology, chemical biology, cell therapy, cell and organ transplantation, developmental biology, ecology, endocrinology, epidemiology, evolution, genetics, gene therapy, genomics, gerontology, immunology, infectious diseases, microbiology, molecular biology, nephrology, neurology, ophthalmology, pediatrics, pharmacology, physiology, plant biology, psychiatry, public health, structural biology, surgery, urology, drug discovery, molecular therapeutics, epidemiology, carcinogenesis, inflammation, pain, nutrition, reproduction, virology, toxicology, pathology, dermatology, gastroenterology, musculoskeletal studies, pregnancy, pulmonary studies, breast cancer, cardiovascular studies, cerebrospinal research, allergy and asthma studies, hepatology, atherosclerosis, diabetes studies, hematology, oncology, osteoporosis studies, rheumatology studies, vaccine studies, circadian rhythms studies, proteome studies, respiratory research, thrombosis studies, anti-viral and anti-microbial and anti-parasite studies gene regulation studies, cell culture studies of all kinds, organ culture and transplant studies of all kinds, protein production studies, in vitro and in vivo cell and organ growth and differentiation studies of all kinds, and combinations thereof. The disclosure provides a method wherein the purified air has reduced biological contaminants compared to non-purified air, wherein the biological contaminants are selected from the group consisting of viral contaminants, bacterial contaminants, fungal contaminants, parasitic contaminants, and combinations thereof. The disclosure provides a method wherein the purified air has reduced viral contaminants compared to non-purified air, wherein the viral contaminants are selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, Astrovirus, HAV, Rotavirus, and combinations thereof. The disclosure provides a method wherein the purified air has reduced bacterial contaminants compared to non-purified air, wherein the bacterial contaminants are selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof. The disclosure provides a method wherein the purified air has reduced fungal contaminants compared to non-purified air, wherein the fungal contaminants are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum; and combinations thereof. The disclosure provides a method wherein the purified air has reduced parasitic contaminants compared to non-purified air, wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium; and combinations thereof. The disclosure provides a method wherein the purified air has reduced chemical contaminants compared to non-purified air, wherein the chemical contaminants are selected from the group consisting of tobacco smoke, engine exhaust, volatile organic compounds from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, the like, and combinations thereof. VOCs include organic solvents, certain paint additives, aerosol spray can propellants, fuels (such as gasoline, and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone. The disclosure provides a method wherein the purified air has reduced particulate contaminants compared to non-purified air, wherein the particulate contaminants are selected from the group consisting of indoor allergens, dust mite feces, dander, cockroach-derived allergens, and combinations thereof. The disclosure provides a method wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier which is an in-room unit that is an in-ceiling unit that purifies in-room air, an air purifier which is an in-room unit that is a ceiling unit that purifies in-room air, and combinations thereof.

### BRIEF DESCRIPTION OF SEVERAL VIEWS OF THE DRAWINGS

Certain embodiments of the disclosure will be described in conjunction with the following drawings in which like reference numerals designate like elements and wherein:

FIG. 1 is a top view of an air purifier according to the present disclosure.

FIG. 2 is a side view of an air purifier according to the present disclosure.

FIG. 3 is an internal view of the air purifier along the plane defined by section line A-A of FIG. 1.

FIG. 4 is an internal view of the air purifier along the plane defined by section line B-B of FIG. 2.

FIG. 5 is a Chart showing Clinical Outcome Data: Pre- and Post-comprehensive Control of Ambient Air.

FIG. 6 is a chart showing Log Reduction of Airborne Biological Pathogens.

FIG. 7 is a chart showing the Viral Kill.

FIG. 8 is a chart showing HEPA Filtration Versus LifeAire Purification.

FIG. 9 is a chart showing the impact of ambient air quality.

FIG. 10 is a chart showing Analysis of Clinical Pregnancy Rate, TVOC, and Biological.

FIG. 11 is a chart showing the survival period for various pathogens.

FIG. 12 is chart showing Results for Viable Airborne and Surface Bacteria and Fungi, and VOC Load in Each Zone (AHU-HEPA, MIXED, and LSAR); Legend: Air handling unit (“AHU”); Viable fungi by air (“VFBA”) (CFU/m3); Viable fungi by swab (“VFBS”) (CFU/in2); Viable bacteria by air (“VBBA”) (CFU/m3); Viable bacteria by swab (“VBBS”)(CFU/in2); Volatile organic compounds (“VOC”) (ppb); Particulates (“PT”) (mg/m3).

### DETAILED DESCRIPTION OF THE INVENTION

The air purification technology, system, and methods of the disclosure will reduce the levels of airborne biological, chemical, and particulate contamination and thus improve outcomes in all areas of, for example, basic, applied, commercial, industrial, biological, and chemical research and manufacturing, as well as hospital settings, and LTCs, as set forth herein.

The phrase “chemical or biological interaction or reaction” is understood to mean an interaction with the contaminant through either chemical or biological processes that renders the contaminant incapable of causing harm. Examples of this are reduction, oxidation, chemical denaturing, and physical damage to microorganisms, bio-molecules, ingestion, and encasement.

### Categories of Air Contamination

The Categories of Air Contamination which can be reduced or eliminated by the air purification technology, system, and methods of the disclosure include, for example, VOCs: ethanol, styrene, toluene, aldehydes; Viable Particulates: biological and viral particulates, microbial and fungal pathogens; Nonviable Particulates: classification of ISO and Class Rating, non-infectious but serve as “vehicles” for infectious viable particulates

Methods of preferred embodiments can be employed to abate any of the substances discussed herein, but the methods are particularly preferred for biological contaminants, chemical contaminants, particulate contaminants, pathogens, molds, allergens, and VOCs.

### Pathogens

Pathogens that can be controlled by the system and methods of the disclosure include, but are not limited to, Anthrax (Bacillus anthracis); Botulism (Clostridium botulinum toxin); Brucella species (brucellosis); Brucellosis (Brucella species); Burkholderia mallei (glanders); Burkholderia pseudomallei (melioidosis); Chlamydia psittaci (psittacosis); Cholera (Vibrio cholerae); Clostridium botulinum toxin (botulism); Clostridium perfringens (Epsilon toxin); Coxiella burnetii (Q fever); E. coli O157:H7 (Escherichia coli); Emerging infectious diseases such as Nipah virus and hantavirus; Norwalk virus; Severe Acute Respiratory Syndrome (SARS); Acquired Immune Deficiency Syndrome (AIDS) virus; Human Immunodeficiency Virus (HIV); Epsilon toxin of Clostridium perfringens; Escherichia coli O157:H7 (E. coli); Food safety threats (e.g., Salmonella species, Escherichia coli O157:H7, Shigella); Francisella tularensis (tularemia); Glanders (Burkholderia mallei); Melioidosis (Burkholderia pseudomallei); Plague (Yersinia pestis); Psittacosis (Chlamydia psittaci); Q fever (Coxiella burnetii); Ricin toxin from Ricinus communis (castor beans); Rickettsia prowazekii (typhus fever); Salmonella species (salmonellosis); Salmonella typhi (typhoid fever); Salmonellosis (Salmonella species); Shigella (shigellosis); Shigellosis (Shigella); Smallpox (Variola major); Staphylococcal enterotoxin B; Tularemia (Francisella tularensis); Typhoid fever (Salmonella Typhi); Typhus fever (Rickettsia prowazekii); Variola major (smallpox); Vibrio cholerae (cholera); Viral encephalitis (alphaviruses [e.g., Venezuelan equine encephalitis, eastern equine encephalitis, western equine encephalitis]); Viral hemorrhagic fevers (filoviruses [e.g., Ebola, Marburg] and arenaviruses [e.g., Lassa, Machupo]); Water safety threats (e.g., Vibrio cholerae, Cryptosporidium parvum); and Yersinia pestis (plague); Non-limiting examples of microbes and infection types and biochemical activities that cause or can cause reductions in performance include ventilator-associated pneumonia, Staphylococcus aureus (including methicillin resistant strains), Candida albicans, Pseudomonas aeruginosa, Acinetobacter baumannii, Stenotrophomonas maltophilia, E. coli O157:H7, Clostridium difficile, Tuberculosis, Urinary tract infections, pneumonia, Gastroenteritis, Enterococcus (including Vancomycin-resistant strains), Legionnaires' disease, Puerperal fever, antibiotic resistance, specific metabolic pathways or enzymes in them, and specific types of genes or gene segments. Microbial contamination within a factory facility generally leads to a reduction in performance, which causes loss of product, and increased wastage. Non-limiting examples of performance reducing microbial contamination, and related illnesses, include E. coli, including O157:H7, botulism, bovine spongiform encephalopathy, Listeria, Campylobacter, norovirus, Trichinosis, Staphylococcus aureus, Salmonella, and the genes and biochemical activities uniquely or specifically associated with them.

Sources of air contamination include outside air, recirculated air, HVAC components, staff, procedures and protocols. Greater than 90% of all surface fomites and pathogens originate from the air.

### Biological Contaminants

Biological Contaminants that can be controlled by the systems and methods of the disclosure include, but are not limited to viral contaminants, bacterial contaminants, fungal contaminants, parasitic contaminants, and combinations thereof.

Viruses—A non-exhaustive list of viruses and their species which can be removed, prevented, and/or treated by the air purification system and methods of the disclosure include, for example: Abadina virus (Reoviridae), Abelson murine leukemia virus (Retroviridae), Abras virus (Bunyaviridae), Absettarov virus (Flaviviridae), Abu Hammad virus (Bunyaviridae), Abu Mina virus (Bunyaviridae), Acado virus (Reoviridae), Acara virus (Bunyaviridae), Acciptrid herpesvirus (Herpesviridae), Acheta domestica densovirus (Parvoviridae), Acrobasis zelleri entomopoxvirus (Poxviridae), Adelaide River virus (Rhabdoviridae), Adeno-associated virus (Parvoviridae), Aedes aegypti densovirus (Parvoviridae), Aedes aegypti entomopoxvirus (Poxviridae), Aedes albopictus densovirus (Parvoviridae), Aedes pseudoscutellaris densovirus (Parvoviridae), African green monkey cytomegalovirus (Herpesviridae), African green monkey HHV-like virus (Herpesviridae), African green monkey polyomavirus (Papovaviridae), African horse sickness viruses (Reoviridae), African swine fever virus, African swine fever-like viruses, AG-virus (Bunyaviridae), AG-virus, (Bunyaviridae), Agaricus bisporus virus, Aguacate virus (Bunyaviridae), Ahlum water-borne virus (Tombusviridae), Aino virus (Bunyaviridae), Akabane virus (Bunyaviridae), AKR (endogenous) murine leukemia virus (Retroviridae), Alajuela virus (Bunyaviridae), Alcelaphine herpesvirus (Herpesviridae), Alenquer virus (Bunyaviridae), Aleutian disease virus (Parvoviridae), Aleutian mink disease virus (Parvoviridae), Alfuy virus (Flaviviridae), Allerton virus (Herpesviridae), Allitrich herpesvirus (Herpesviridae), Allomyces arbuscula virus, Almeirim virus (Reoviridae), Almpiwar virus (Rhabdoviridae), Altamira virus, (Reoviridae), Amapari virus (Arenaviridae), American ground squirrel herpesvirus, (Herpesviridae), Amsacta moorei entomopoxvirus (Poxviridae), Amyelosis chronic stunt virus (Caliciviridae), Ananindeua virus (Bunyaviridae), Anatid herpesvirus (Herpesviridae), Andasibe virus (Reoviridae), Anhanga virus (Bunyaviridae), Anhembi virus (Bunyaviridae), Anomala cuprea entomopoxvirus (Poxviridae), Anopheles A virus (Bunyaviridae), Anopheles virus (Bunyaviridae), Antequera virus (Bunyaviridae), Aotine herpesvirus (Herpesviridae), Apeu virus (Bunyaviridae), Aphodius tasmaniae entomopoxvirus (Poxviridae), Apoi virus (Flaviviridae), Aransas Bay virus (Bunyaviridae), Arbia virus (Bunyaviridae), Arboledas virus (Bunyaviridae), Arbroath virus (Reoviridae), Argentine turtle herpesvirus (Herpesviridae), Arkonam virus (Reoviridae), Aroa virus (Flaviviridae), Arphia conspersa entomopoxvirus (Poxviridae), Aruac virus (Rhabdoviridae), Arumowot virus (Bunyaviridae), Asinine herpesvirus (Herpesviridae), Atlantic cod ulcus syndrome virus (Rhabodoviridae), Atlantic salmon reovirus Australia (Reoviridae), Atlantic salmon reovirus Canada (Reoviridae), Atlantic salmon reovirus USA (Reoviridae), Atropa belladorma virus (Rhabdoviridae), Aucuba bacilliform virus, Badnavirus, Aujeszky's disease virus (Herpesviridae), Aura virus (Togaviridae), Auzduk disease virus (Poxviridae), Avalon virus (Bunyaviridae), Avian adeno-associated virus (Parvoviridae), Avian carcinoma, Mill Hill virus (Retroviridae), Avian encephalomyelitis virus (Picornaviridae), Avian infectious bronchitis virus (Coronaviridae), Avian leukosis virus—RSA (Retroviridae), Avian myeloblastosis virus (Retroviridae), Avian myelocytomatosis virus (Retroviridae), Avian nephrites virus (Picornaviridae), Avian paramyxovirus (Paramyxoviridae), Avian reovirus (Reoviridae), B virus (Parvoviridae), B-lymphotropic papovavirus (Papovaviridae), Babahoya virus (Bunyaviridae), Babanki virus (Togaviridae), Baboon herpesvirus (Herpesviridae), Baboon polyomavirus (Papovaviridae), Bagaza virus (Flaviviridae), Bahia Grande virus (Rhabdoviridae), Bahig virus (Bunyaviridae), Bakau virus (Bunyaviridae), Baku virus (Reoviridae), Bald eagle herpesvirus (Herpesviridae), Bandia virus (Bunyaviridae), Bangoran virus (Rhabdoviridae), Bangui virus (Bunyaviridae), Banzi virus (Flaviviridae), Barmah Forest virus (Togaviridae), Barranqueras virus (Bunyaviridae), Barur virus (Rhabdoviridae), Batai virus (Bunyaviridae), Batarna virus (Bunyaviridae), Batken virus (Bunyaviridae), Bauline virus (Reoviridae), Beak and feather disease virus (Circoviridae), BeAn virus (Rhabdoviridae), BeAr virus (Bunyaviridae), Bebaru virus (Togaviridae), Belem virus (Bunyaviridae), Belmont virus ((Bunyaviridae)), Belterra virus (Bunyaviridae), Benevides virus (Bunyaviridae), Benfica virus (Bunyaviridae), Berne virus, (Coronaviridae), Berrimah virus (Rhabdoviridae), Bertioga virus (Bunyaviridae), Bhanja virus (Bunyaviridae), Bimbo virus (Rhabdoviridae), Bimiti virus (Bunyaviridae), Birao virus (Bunyaviridae), BivensArm virus (Rhabdoviridae), BK virus (Papovaviridae), Bluetongue viruses (Reoviridae), Bobaya virus (Bunyaviridae), Bobia virus (Bunyaviridae), Bobwhite quail herpesvirus (Herpesviridae), Boid herpesvirus (Herpesviridae), Bombyx mori densovirus (Parvoviridae), Boolarra virus (Nodaviridae), Boraceia virus (Bunyaviridae), Border disease virus (Flaviviridae), Boma disease virus, Botambi virus (Bunyaviridae), Boteke virus, (Rhabdoviridae), Bouboui virus (Flaviviridae), Bovine adeno-associated virus (Parvoviridae), Bovine adenoviruses (Adenoviridae), Bovine astrovirus (Astroviridae), Bovine coronavirus (Coronaviridae), Bovine diarrhea virus (Flaviviridae), Bovine encephalitis herpesvirus (Herpesviridae), Bovine enteric calicivirus (Caliciviridae), Bovine enterovirus (Picornaviridae), Bovine ephemeral fever virus (Rhabdoviridae), Bovine herpesvirus (Herpesviridae), Bovine immunodeficiency virus (Retroviridae), Bovine leukemia virus (Retroviridae), Bovine mamillitis virus (Herpesviridae), Bovine papillomavirus (Papovaviridae), Bovine papular stomatitis virus (Poxviridae), Bovine parainfluenza virus (Paramyxoviridae), Bovine parvovirus (Parvoviridae), Bovine polyomavirus (Papovaviridae), Bovine Respiratory Syncytial Virus (Paramyxoviridae), Bovine rhinovirus (Picornaviridae), Bovine syncytial virus (Retroviridae), Bozo virus (Bunyaviridae), Broadhaven virus (Reoviridae), Bruconha virus (Bunyaviridae), Brus Laguna virus (Bunyaviridae), Budgerigar fledgling disease virus (Papovaviridae), Buenaventura virus (Bunyaviridae), Buffalopox virus (Poxviridae), Buggy Creek virus (Togaviridae), Bujaru virus (Bunyaviridae), Bukalasa bat virus (Flaviviridae), Bunyamwera virus (Bunyaviridae), Bunyip creek virus (Reoviridae), Bushbush virus (Bunyaviridae), Bussuquara virus (Flaviviridae), Bwamba virus (Bunyaviridae), Cache Valley virus (Bunyaviridae), Cacipacore virus (Flaviviridae), Caddo Canyon virus (Bunyaviridae), Caimito virus (Bunyaviridae), Calchaqui virus (Rhabdoviridae), California encephalitis virus (Bunyaviridae), California harbor sealpox virus (Poxviridae), Callistephus chinensis chlorosis virus (Rhabdoviridae), Callitrichine herpesvirus (Herpesviridae), Camel contagious ecthyma virus (Poxviridae), Camelpox virus (Poxviridae), Camptochironomus tentans entomopoxvirus (Poxviridae), Cananeia virus (Bunyaviridae), Canarypox virus (Poxviridae), Candiru virus (Bunyaviridae), Canid herpesvirus (Herpesviridae), Caninde virus (Reoviridae), Canine adeno-associated virus (Parvoviridae), Canine adenovirus (Adenoviridae), Canine calicivirus (Caliciviridae), Canine coronavirus (Coronaviridae), Canine distemper virus (Paramyxoviridae), Canine herpesvirus (Herpesviridae), Canine minute virus (Parvoviridae), Canine oral papillomavirus (Papovaviridae), Canine parvovirus (Parvoviridae), Canna yellow mottle virus (Badnavirus), Cape Wrath virus (Reoviridae), Capim virus (Bunyaviridae), Caprine adenovirus (Adenoviridae), Caprine arthritis encephalitis virus (Retroviridae), Caprine herpesvirus (Herpesviridae), Capuchin herpesvirus AL- (Herpesviridae), Capuchin herpesvirus AP- (Herpesviridae), Carajas virus (Rhabdoviridae), Caraparu virus (Bunyaviridae), Carey Island virus (Flaviviridae), Casphalia extranea densovirus (Parvoviridae), Catu virus (Bunyaviridae), Caviid herpesvirus ((Herpesviridae)), CbaAr virus (Bunyaviridae), Cebine herpesvirus (Herpesviridae), Cercopithecine herpesvirus (Herpesviridae), Cervid herpesvirus (Herpesviridae), CG-virus (Bunyaviridae), Chaco virus (Rhabdoviridae), Chagres virus (Bunyaviridae), Chamois contagious ecthyma virus (Poxviridae), Chandipura virus (Rhabdoviridae), Changuinola virus (Reoviridae), Charleville virus (Rhabdoviridae), Chelonid herpesvirus (Herpesviridae), Chelonid herpesvirus (Herpesvirzdae), Chelonid herpesvirus (Herpesviridae), Chenuda virus (Reoviridae), Chick syncytial virus (Retroviridae), Chicken anemia virus (Circoviridae), Chicken parvovirus (Paruoviridae), Chikungunya virus (Togaviridae), Chilibre virus (Bunyaviridae), Chim virus (Bunyaviridae), Chimpanzee herpesvirus (Herpesviridae), Chironomus attenuatus entomopoxvirus (Poxviridae), Chironomus luridus entomopoxvirus (Poxviridae), Chironomus plumosus erltomopoxvirus (Poxviridae), Chobar Gorge virus (Reoviridae), Choristoneura biennis entomopoxvirus (Poxviridae), Choristoneura conflicta entomopoxvirus (Poxviridae), Choristoneura diversuma entomopoxvirus (Poxviridae), Chorizagrotis auxiliars entomopoxvirus (Poxviridae), Chub reovirus Germany (Reoviridae), Ciconiid herpesvirus (Herpesviridae), Clo Mor virus (Bunyaviridae), CoAr-virus (Bunyaviridae), Coastal Plains virus (Rhabdoviridae), Cocal virus (Rhabdoviridae), Coital exanthema virus (Herpesviridae), ColAn-virus (Bunyaviridae), Colocasia bobone disease virus, (Rhabdoviridae), Colorado tick fever virus, (Reoviridae), Columbia SK virus, (Picornaviridae), Columbid herpesvirus, (Herpesviridae), Connecticut virus, (Rhabdoviridae), Contagious ecthyma virus, (Poxviridae), Contagious pustular dermatitis virus, (Poxviridae), Corfu virus, (Bunyaviridae), Corriparta virus, (Reoviridae), Cotia virus, (Poxviridae), Cowpox virus, (Poxviridae), Crimean-Congo hemorrhagic fever virus, (Bunyaviridae), CSIRO village virus, (Reoviridae), Cynara virus, (Rhabdoviridae), Cyprinid herpesvirus, (Herpesviridae), Dabakala virus, (Bunyaviridae), D'Aguilar virus, (Reoviridae), Dakar bat virus, (Flaviviridae), DakArk virus, (Rhabdoviridae), Deer papillomavirus, (Papovaviridae), Demodema boranensis entomopoxvirus, (Poxviridae), Dengue virus, (Flaviviridae), Dengue virus group, (Flaviviridae), Dependovirus, (Parvoviridae), Dera Ghazi Khan virus, (Bunyaviridae), Dera Ghazi Khan virus Group, (Bunyaviridae), Dermolepida albohirtum entomopoxvirus, (Poxviridae), Dhori virus, (Orthomyxoviridae), Diatraea saccharalis densovirus, (Parvoviridae), Dobrava-Belgrade virus, (Bunyaviridae), Dolphin distemper virus, (Paramyxoviridae), Dolphinpox virus, (Poxviridae), Douglas virus, (Bunyaviridae), Drosophila C virus, (Picornaviridae), Dry Tortugas virus, (Bunyaviridae), duck adenovirus, (Adenoviridae), Duck adenovirus, (Adenoviridae), Duck astrovirus, (Astroviridae), Duck hepatitis B virus, (Hepadnaviridae), Duck plague herpesvirus syn. anatid herpesvirus, (Herpesviridae), Dugbe virus, (Bunyaviridae), Duvenhage virus, (Rhabdoviridae), Eastern equine encephalitis virus, (Togaviridae), Ebola virus Filoviridae, Echinochloa hoja blanca virus; Genus Tenuivirus, Echinochloa ragged stunt virus, (Reoviridae), ectromelia virus, (Poxviridae), Edge Hill virus, (Flaviviridae), Egtved virus syn. viral hemorrhagic septicemia virus, (Rhabdoviridae), Elapid herpesvirus, (Herpesviridae), Elephant loxondontal herpesvirus, (Herpesviridae), Elephant papillomavirus, (Papovaviridae), Elephantid herpesvirus, (Herpesviridae), Ellidaey virus, (Reoviridae), Embu virus, (Poxviridae), Encephalomyocarditis virus, (Picornaviridae), Enseada virus, (Bunyaviridae), Entamoeba virus, (Rhabdoviridae), Entebbe bat virus, (Flaviviridae), Epizootic hemorrhagic disease viruses, (Reoviridae), Epstein-Barr virus, (Herpesviridae), Equid herpesvirus, (Herpesviridae), Equid herpesvirus, (Nerpesviridae), Equid herpesvirus, (Herpesviridae), Equine abortion herpesvirus, (Herpesviridae), Equine adeno-associated virus, (Parvoviridae), Equine adenovirus, (Adenoviridae), Equine arteritis virus, (Arterivirus), Equine cytomegalovirus, (Herpesviridae), Equine encephalosis viruses, (Reoviridae), Equine herpesvirus, (Herpesviridae), Equine infectious anemia virus, (Retroviridae), Equine papillomavirus, (Papovaviridae), Equine rhinopneumonitis virus, (Herpesviridae), Equine rhinovirus, (Picornaviridae), Eret-virus, (Bunyaviridae), Erinaceid herpesvirus, (Herpesviridae), Erve virus, (Bunyaviridae), Erysimum latent virus, Tymovirus, Esocid herpesvirus, (Herpesviridae), Essaouira virus, (Reoviridae), Estero Real virus, (Bunyaviridae), Eubenangee virus, (Reoviridae), Euonymus fasciation virus, (Rhabdoviridae), European bat virus, (Rhabdoviridae), European brown hare syndrome virus, (Caliciviridae), European elk papillomavirus, (Papovaviridae), European ground squirrel cytomegalovirus, (Herpesviridae), European hedgehog herpesvirus, (Herpesviridae), Everglades virus, (Togaviridae), Eyach virus, (Reoviridae), Facey's Paddock virus, (Bunyaviridae), Falcon inclusion body disease, (Herpesviridae), Falconid herpesvirus, (Herpesviridae), Farallon virus, (Bunyaviridae), Felid herpesvirus, (Herpesviridae), Feline calicivirus, (Caliciviridae), Feline herpesvirus, (Herpesviridae), Feline immunodeficiency virus, (Retroviridae), Feline infectious peritonitis virus, (Coronaviridae), Feline leukemia virus, (Retroviridae), Feline parlleukopenia virus, (Parvoviridae), Feline parvovirus, (Parvoviridae), Feline syncytial virus, (Retroviridae), Feline viral rhinotracheitis virus, (Herpesviridae), Fetal rhesus kidney virus, (Papovaviridae), Field mouse herpesvirus, (Herpesviridae), Figulus subleavis entomopoxvirus, (Poxviridae), Fiji disease virus, (Reoviridae), Fin V-virus, (Bunyaviridae), Finkel-Biskis-Jinkins murine sarcoma virus, (Retroviridae), Flanders virus, (Rhabdoviridae), Flexal virus, (Arenaviridae), Flock house virus, Nodaviridae, Foot-and-mouth disease virus A, (Picornaviridae), Foot-and-mouth disease virus ASIA, (Picornaviridae), Foot-and-mouth disease virus, (Picornaviridae), Forecariah virus, (Bunyaviridae), Fort Morgan virus, (Togaviridae), Fort Sherman virus, (Bunyaviridae), Foula virus, (Reoviridae), Fowl adenoviruses, (Adenoviridae), Fowl calicivirus, (Caliciviridae), Fowlpox virus, (Poxviridae), Fraser Point virus, (Bunyaviridae), Friend murine leukemia virus, (Retroviridae), Frijoles virus, (Bunyaviridae), Frog herpesvirus, (Herpesviridae), Fromede virus, (Reoviridae), Fujinami sarcoma virus, (Retroviridae), Fukuoka virus, (Rhabdoviridae), Gabek Forest virus, (Bunyaviridae), Gadget's Gully virus, (Flaviviridae), Galleria mellonella densovirus, (Parvoviridae), Gallid herpesvirus, (Herpesviridae), Gamboa virus, (Bunyaviridae), Gan Gan virus, (Bunyaviridae), Garba virus, (Rhabdoviridae), Gardner-Arnstein feline sarcoma virus, (Retroviridae), Geochelone carbonaria herpesvirus, (Herpesviridae), Geochelone chilensis herpesvirus, (Herpesviridae), Geotrupes sylvaticus entomopoxvirus, (Poxviridae), Gerbera symptomless virus, (Rhabdoviridae), Germiston virus, (Bunyaviridae), Getah virus, (Togaviridae), Gibbon ape leukemia virus, (Retroviridae), Ginger chlorotic fleckvirus, Sobemovirus, Glycine mottle virus, Tombusviridae, Goat herpesvirus, (Herpesviridae), Goatpox virus, (Poxviridae), Goeldichironomus holoprasimus entomopoxvirus, (Poxviridae), Golden shiner reovirus, (Reoviridae), Gomoka virus, (Reoviridae), Gomphrena virus, (Rhabdoviridae), Gonometa virus, (Picornaviridae), Goose adenoviruses, (Adenoviridae), Goose parvovirus, (Parvoviridae), Gordil virus, (Bunyaviridae), Gorilla herpesvirus, (Herpesviridae), Gossas virus, (Rhabdoviridae), Grand Arbaud virus, (Bunyaviridae), Gray Lodge virus, (Rhabdoviridae), Gray patch disease agent of green sea turtle, (Herpesviridae), Great Island virus, (Reoviridae), Great Saltee Island virus, (Reoviridae), Great Saltee virus, (Bunyaviridae), Green iguana herpesvirus, (Herpesviridae), Green lizard herpesvirus, (Herpesviridae), Grey kangaroopox virus, (Poxviridae), Grimsey virus, (Reoviridae), Ground squirrel hepatitis B virus, (Hepadnaviridae), GroupA-K rotaviruses, (Reoviridae), Gruid herpesvirus, (Herpesviridae), GUU-virus, (Bunyaviridae), Guajara virus, (Bunyaviridae), Guama virus, (Bunyaviridae), Guanarito virus, (Arenaviridae), Guaratuba virus, (Bunyaviridae), Guaroa virus, (Bunyaviridae), Guinea pig cytomegalovirus, (Herpesviridae), Guinea pig herpesvirus, (Herpesviridae), Guinea pig type C oncovirus, (Retroviridae), Gumbo Limbo virus, (Bunyaviridae), Gurupi virus, (Reoviridae), H-virus, (Parvoviridae), H virus, (Bunyaviridae), Hamster herpesvirus, (Herpesviridae), Hamster polyomavirus, (Papovaviridae), Hantaan virus, (Bunyaviridae), Hanzalova virus, (Flaviviridae), Hardy-Zuckerman feline sarcoma virus, (Retroviridae), Hare fibroma virus, (Poxviridae), Hart Park virus, (Rhabdoviridae), Hartebeest herpesvirus, (Herpesviridae), Harvey murine sarcoma virus, (Retroviridae), Hazara virus, (Bunyaviridae), HB virus, (Parvoviridae), Hepatitis virus, (Picornaviridae), Hepatitis virus, (Hepadnaviridae), Hepatitis virus, (Flaviviridae), Herpesvirus M, (Herpesviridae), Herpesvirus papio, (Herpesviridae), Herpesvirus platyrrhinae type, (Herpesviridae), Herpesvirus pottos, (Herpesviridae), Herpesvirus saimiri, (Herpesviridae), Herpesvirus salmonis, (Herpesviridae), Herpesvirus sanguinus, (Herpesviridae), Herpesvirus scophthalmus, (Herpesviridae), Herpesvirus sylvilagus, (Herpesviridae), Herpesvirus T, (Herpesviridae), Herpesvirus tarnarinus, (Herpesviridae), Highlands J virus, (Togaviridae), Hirame rhabdovirus, (Rhabdoviridae), Hog cholera virus, (Flaviviridae), HoJo virus, (Bunyaviridae), Hepatitis delta virus, Satellites, Deltavirus, Hsiung Kaplow herpesvirus, (Herpesviridae), Hepatitis E virus, (Caliciviridae), Hepatopancreatic parvo-like virus of shrimps, (Parvoviridae), Heron hepatitis B virus, (Hepadnaviridae), Herpes ateles, (Herpesviridae), Herpes simiae virus, (Herpesviridae), Herpes simplex virus, (Herpesviridae), Herpes virus B, (Herpesviridae), Herpesvirus aotus, (Herpesviridae), Herpesvirus ateles strain, (Herpesviridae), Herpesvirus cuniculi, (Herpesviridae), Herpesvirus cyclopsis, (Herpesviridae), Huacho virus, (Reoviridae), Hughes virus, (Bunyaviridae), Human adenoviruses, (Adenoviridae), Human astrovirus, (Astroviridae), Human calicivirus, (Caliciviridae), Human caliciviruses, (Caliciviridae), Human coronavirus E, (Coronaviridae), Human coronavirus OC, (Coronaviridae), Human coxsackievirus, (Picornaviridae), Human cytomegalovirus, (Herpesviridae), Human echovirus, (Picornaviridae), Human enterovirus, (Picornaviridae), Human foamy virus, (Retroviridae), Human herpesvirus, (Herpesviridae), Human herpesvirus, Nerpesviridae, Human herpesvirus, (Herpesviridae), Human immunodeficiency virus, (Retroviridae), Human papillomavirus, (Papovaviridae), Human parainfluenza virus, (Paramyxoviridae), Human poliovirus, (Picornaviridae), Human Respiratory Syncytial Virus, (Paramyxoviridae), Human rhinovirus, (Picornaviridae), Human spumavirus, (Retroviridae), Human T-lymphotropic virus, (Retroviridae), Humpty Doo virus, (Rhabdoviridae), HV-virus, (Bunyaviridae), Hypr virus, (Flaviviridae), Laco virus, (Bunyaviridae), Ibaraki virus, (Reoviridae), Icoaraci virus, (Bunyaviridae), Ictalurid herpesvirus, (Herpesviridae), Len virus, (Reoviridae), Ife virus, (Reoviridae), Iguanid herpesvirus, (Herpesviridae), Besha virus, (Bunyaviridae), Ilheus virus, (Flaviviridae), Inclusion body rhinitis virus, (Herpesviridae), Infectious bovine rhinotracheitis virus, (Herpesviridae), Infectious bursal disease virus, Birnaviridae, Infectious hematopoietic necrosis virus, (Rhabdoviridae), Infectious laryngotracheitis virus, (Herpesviridae), Infectious pancreatic necrosis virus, Birnavirzdae, InfluenzaA virus (A/PR//(HN), (Orthomyxoviridae), Influenza B virus (B/Lee/), (Orthomyxoviridae), Influenza C virus (C/California/), (Orthomyxoviridae), Ingwavuma virus, (Bunyaviridae), mini virus, (Bunyaviridae), Inkoo virus, (Bunyaviridae), Inner Frame virus, (Reoviridae), Ippy virus, (Arenaviridae), Irituia virus, (Reoviridae), Isfahan virus, (Rhabdoviridae), Israel turkey meningoencephalitis virus, (Flaviviridae), Issyk-Kul virus, (Bunyaviridae), Itaituba virus, (Bunyaviridae), Itaporanga virus, (Bunyaviridae), Itaqui virus, (Bunyaviridae), Itimirirn virus, (Bunyaviridae), Itupiranga virus, (Reoviridae), Jaagsiekte virus, (Retroviridae), Jacareacanga virus, (Reoviridae), Jamanxi virus, (Reoviridae), Jamestown Canyon virus, (Bunyaviridae), Japanaut virus, (Reoviridae), Japanese encephalitis virus, (Flaviviridae), Jan virus, (Reoviridae), JC virus, (Papovaviridae), Joa virus, (Bunyaviridae), Joinjakaka virus, (Rhabdoviridae), Juan Diaz virus, (Bunyaviridae), Jugra virus, (Flaviviridae), Juncopox virus, (Poxviridae), Junin virus, (Arenaviridae), Junonia coenia densovirus, (Parvoviridae), Jurona virus, (Rhabdoviridae), Jutiapa virus, (Flaviviridae), K virus, (Papovaviridae), K virus, (Bunyaviridae), Kachemak Bay virus, (Bunyaviridae), Kadarn virus, (Flaviviridae), Kaeng Khoi virus, (Bunyaviridae), Kaikalur virus, (Bunyaviridae), Kairi virus, (Bunyaviridae), Kaisodi virus, (Bunyaviridae), Kala Iris virus, (Reoviridae), Kamese virus, (Rhabdoviridae), Karnmavanpettai virus, (Reoviridae), Kannamangalam virus, (Rhabdoviridae), Kao Shuan virus, (Bunyaviridae), Karimabad virus, (Bunyaviridae), Karshi virus, (Flaviviridae), Kasba virus, (Reoviridae), Kasokero virus, (Bunyaviridae), Kedougou virus, (Flaviviridae), Kemerovo virus, (Reoviridae), Kenai virus, (Reoviridae), Kennedya virus Y, Potyviridae, Kern Canyon virus, (Rhabdoviridae), Ketapang virus, (Bunyaviridae), Keterah virus, (Bunyaviridae), Keuraliba virus, (Rhabdoviridae), Keystone virus, (Bunyaviridae), Kharagysh virus, (Reoviridae), Khasan virus, (Bunyaviridae), Kilham rat virus, (Parvoviridae), Kimberley virus, (Rhabdoviridae), Kindia virus, (Reoviridae), Kinkajou herpesvirus, (Herpesviridae), Kirsten murine sarcoma virus, (Retroviridae), Kismayo virus, (Bunyaviridae), Klamath virus, (Rhabdoviridae), Kokobera virus, (Flaviviridae), Kolongo virus, (Rhabdoviridae), Koolpinyah virus, (Rhabdoviridae), Koongol virus, (Bunyaviridae), Kotonkan virus, (Rhabdoviridae), Koutango virus, (Flaviviridae), Kowanyama virus, (Bunyaviridae), Kumlinge virus, (Flaviviridae), Kunjin virus, (Flaviviridae), Kwatta virus, (Rhabdoviridae), Kyzylagach virus, (Togaviridae), La Crosse virus, (Bunyaviridae), La Joya virus, (Rhabdoviridae), La-Piedad-Michoacan-Mexico virus, (Paramyxoviridae), Lacertid herpesvirus, (Herpesviridae), Lactate dehydrogenase-elevating virus, (Arterivirus), Lagos bat virus, (Rhabdoviridae), Lake Clarendon virus, (Reoviridae), Lake Victoria cormorant herpesvirus, (Herpesviridae), Langat virus, Flaviviridae, Langur virus, (Retroviridae), Lanjan virus, (Bunyaviridae), Lapine parvovirus, (Parvoviridae), Las Maloyas virus, (Bunyaviridae), Lassa virus, (Arenaviridae), Lato river virus, (Tombusviridae), Le Dantec virus, (Rhabdoviridae), Leanyer virus, (Bunyaviridae), Lebombo virus, (Reoviridae), Lednice virus, (Bunyaviridae), Lee virus, (Bunyaviridae), Leporid herpesvirus, (Herpesviridae), Leucorrhinia dubia densovirus, (Parvoviridae), Lipovnik virus, (Reoviridae), Liverpool vervet monkey virus, (Herpesviridae), Llano Seco virus, (Reoviridae), Locusta migratona entomopoxvirus, (Poxviridae), Lokem virus, (Bunyaviridae), Lone Star virus, (Bunyaviridae), Lorisine herpesvirus, (Herpesviridae), Louping ill virus, Flaviviridae, Lucke frog herpesvirus, (Herpesviridae), Lum virus, (Parvoviridae), Lukuni virus, (Bunyaviridae), Lumpy skin disease virus, (Poxviridae), Lundy virus, (Reoviridae), Lymantria dubia densovirus, (Parvoviridae), Lymphocytic choriomeningitis virus, (Arenaviridae), Machupo virus, (Arenaviridae), Macropodid herpesvirus (Herpesviridae), Madrid virus, (Bunyaviridae), Maguari virus, (Bunyaviridae), Main Drain virus, (Bunyaviridae), Malakal virus, (Rhabdoviridae), Malignant catarrhal fever virus of European cattle, (Herpesviridae), Malpais Spring virus, (Rhabdoviridae), Malva silvestris virus, (Rhabdoviridae), Manawa virus, (Bunyaviridae), Manawatu virus, (Nodaviridae), Manitoba virus, (Rhabdoviridae), Manzanilla virus, (Bunyaviridae), Map turtle herpesvirus, (Herpesviridae), Mapputta virus, (Bunyaviridae), Maprik virus, (Bunyaviridae), Maraba virus, (Rhabdoviridae), Marburg virus, (Filoviridae), Marco virus, (Rhabdoviridae), Marek's disease herpesvirus, (Herpesviridae), Marituba virus, (Bunyaviridae), Marmodid herpesvirus, (Herpesviridae), Marmoset cytomegalovirus, (Herpesviridae), Marmoset herpesvirus, (Herpesviridae), Marmosetpox virus, (Poxviridae), Marrakai virus, (Reoviridae), Mason-Pfizer monkey virus, (Retroviridae), Masou salmon reovirus, (Reoviridae), Matruh virus, (Bunyaviridae), Matucare virus, (Reoviridae), Mayaro virus, (Togaviridae), Mboke virus, (Bunyaviridae), Meaban virus, (Flaviviridae), Measles (Edmonston) virus, (Paramyxoviridae), Medical Lake macaque herpesvirus, (Herpesviridae), Melanoplus sanguinipes entomopoxvirus, (Poxviridae), Melao virus, (Bunyaviridae), Meleagrid herpesvirus, (Herpesviridae), Melilotus latent virus, (Rhabdoviridae), Melolontha melolontha entomopoxvirus, (Poxviridae), Mengovirus, (Picornaviridae), Mermet virus, (Bunyaviridae), Mice minute virus, (Parvoviridae), Mice pneumotropic virus, (Papovaviridae), Microtus pennsylvanicus herpesvirus, (Herpesviridae), Middelburg virus, (Togaviridae), Miller's nodule virus, (Poxviridae), Mill Door virus, (Reoviridae), Minatitlan virus, (Bunyaviridae), Mink calicivirus, (Caliciviridae), Mink enteritis virus, (Parvoviridae), Minnal virus, (Reoviridae), Mirabilis mosaic virus, Caulimovirus, Mirim virus, (Bunyaviridae), Mitchell river virus, (Reoviridae), Mobala virus, (Arenaviridae), Modoc virus, (Flaviviridae), Moju virus, (Bunyaviridae), Mojui dos Campos virus, (Bunyaviridae), Mokola virus, (Rhabdoviridae), Molluscum contagiosum virus, (Poxviridae), Molluscum-likepox virus, (Poxviridae), Moloney murine sarcoma virus, (Retroviridae), Moloney virus, (Retroviridae), Monkey pox virus, (Poxviridae), Mono Lake virus, (Reoviridae), Montana myotis leukoencephalitis virus, (Flaviviridae), Monte Dourado virus, (Reoviridae), Mopeia virus, (Arenaviridae), Moriche virus, (Bunyaviridae), Mosqueiro virus, (Rhabdoviridae), Mossuril virus, (Rhabdoviridae), Mount Elgon bat virus, (Rhabdoviridae), Mouse cytomegalovirus, (Herpesviridae), Mouse Elberfield virus, (Picornaviridae), Mouse herpesvirus strain, (Herpesviridae), Mouse mammary tumor virus, (Retroviridae), Mouse thymic herpesvirus, (Herpesviridae), Movar herpesvirus, (Herpesviridae), Mucambo virus, (Togaviridae), Mudjinbarry virus, (Reoviridae), Muir Springs virus, (Rhabdoviridae), Mule deerpox virus, (Poxviridae), Multimammate mouse papillomavirus, (Papovaviridae), Mumps virus, (Paramyxoviridae), Murid herpesvirus, (Herpesviridae), Murine adenovirus, (Adenoviridae), Z murine adenovirus, (Adenoviridae), Murine hepatitis virus, (Coronaviridae), Murine herpesvirus, (Herpesviridae), Murine leukemia virus, (Retroviridae), Murine parainfluenza virus, (Paramyxoviridae), Murine poliovirus, (Picornaviridae), Murine polyomavirus, (Papovaviridae), Murray Valley encephalitis virus, (Flaviviridae), Murre virus, (Bunyaviridae), Murutucu virus, (Bunyaviridae), Mykines virus, (Reoviridae), Mynahpox virus, (Poxviridae), Myxoma virus, (Poxviridae), Nairobi sheep disease virus, (Bunyaviridae), Naranjal virus, (Flaviviridae), Nasoule virus, (Rhabdoviridae), Navarro virus, (Rhabdoviridae), Ndelle virus, (Reoviridae), Ndumu virus, (Togaviridae), Neckar river virus, (Tombusviridae), Negishi virus, (Flaviviridae), Nelson Bay virus, New Minto virus, (Rhabdoviridae), Newcastle disease virus, (Paramyxoviridae), Ngaingan virus, (Rhabdoviridae), Ngari virus, (Bunyaviridae), Ngoupe virus, (Reoviridae), Nile crocodilepox virus, (Poxviridae), Nique virus, (Bunyaviridae), Nkolbisson virus, (Rhabdoviridae), Nola virus, (Bunyaviridae), North Clett virus, (Reoviridae), North End virus, (Reoviridae), Northern cereal mosaic virus, (Rhabdoviridae), Northern pike herpesvirus, (Herpesviridae), Northway virus, (Bunyaviridae), NorwaLk virus, (Caliciviridae), Ntaya virus, (Flaviviridae), Nugget virus, (Reoviridae), Nyabira virus, (Reoviridae), Nyamanini virus, Unassigned, Nyando virus, (Bunyaviridae), Oak-Vale virus, (Rhabdoviridae), Obodhiang virus, (Rhabdoviridae), Oceanside virus, (Bunyaviridae), Ockelbo virus, (Togaviridae), Odrenisrou virus, (Bunyaviridae), Oedaleus senegalensis entomopoxvirus, (Poxviridae), Oita virus, (Rhabdoviridae), Okhotskiy virus, (Reoviridae), Okola virus, (Bunyaviridae), Olifantsvlei virus, (Bunyaviridae), Omo virus, (Bunyaviridae), Omsk hemorrhagic fever virus, (Flaviviridae), Onchorhynchus masou herpesvirus, (Herpesviridae), O'nyong-nyong virus, (Togaviridae), Operophtera brurnata entomopoxvirus, (Poxviridae), Orangutan herpesvirus, (Herpesviridae), Orf virus, (Poxviridae), Oriboca virus, (Bunyaviridae), Oriximina virus, (Bunyaviridae), Oropouche virus, (Bunyaviridae), Orungo virus, (Reoviridae), Oryctes rhinoceros virus, Unassigned, Ossa virus, (Bunyaviridae), Ouango virus, (Rhabdoviridae), Oubi virus, (Bunyaviridae), Ourem virus, (Reoviridae), Ovine adeno-associated virus, (Parvoviridae), Ovine adenoviruses, (Adenoviridae), (Astroviridae), Ovine herpesvirus, (Herpesviridae), Ovine pulmonary adenocarcinoma virus, (Retroviridae), Owl hepatosplenitis herpesvirus, (Herpesviridae), P virus, (Bunyaviridae), Pacheco's disease virus, (Herpesviridae), Pacora virus, (Bunyaviridae), Pacui virus, (Bunyaviridae), Pahayokee virus, (Bunyaviridae), Palestina virus, (Bunyaviridae), Palyam virus, (Reoviridae), Pan herpesvirus, (Herpesviridae), Papio Epstein-Barr herpesvirus, (Herpesviridae), Para virus, (Bunyaviridae), Pararnushir virus, (Bunyaviridae), Parana virus, (Arenaviridae), Parapoxvirus of red deer in New Zealand, (Poxviridae), Paravaccinia virus, (Poxviridae), Parma wallaby herpesvirus, (Herpesviridae), Paroo river virus, (Reoviridae), Parrot herpesvirus, (Herpesviridae), Parry Creek virus, (Rhabdoviridae), Pata virus, (Reoviridae), Pates monkey herpesvirus pH delta, (Herpesviridae), Pathum Thani virus, (Bunyaviridae), Patois virus, (Bunyaviridae), Peaton virus, (Bunyaviridae), Percid herpesvirus, (Herpesviridae), Perdicid herpesvirus, (Herpesviridae), Perinet virus, (Rhabdoviridae), Peripianata fuliginosa densovirus, (Parvoviridae), Peste-des-petits-ruminants virus, (Paramyxoviridae), Petevo virus, (Reoviridae), Phalacrocoracid herpesvirus, (Herpesviridae), Pheasant adenovirus, (Adenoviridae), Phnom-Penh bat virus, (Flaviviridae), Phocid herpesvirus, (Herpesviridae), Phocine (seal) distemper virus, (Paramyxoviridae), Pichinde virus, (Arenaviridae), Picola virus, (Reoviridae), Pieris rapae densovirus, (Parvoviridae), Pigeon herpesvirus, (Herpesviridae), Pigeonpox virus, (Poxviridae), Badnavirus Piry virus, (Rhabdoviridae), Pisum virus, (Rhabdoviridae), Pixuna virus, (Togaviridae), Playas virus, (Bunyaviridae), Pleuronectid herpesvirus, (Nerpesviridae), Pneumonia virus of mice, (Paramyxoviridae), Pongine herpesvirus, (Herpesviridae), Pongola virus, (Bunyaviridae), Ponteves virus, (Bunyaviridae), Poovoot virus, (Reoviridae), Porcine adenoviruses, (Adenoviridae), Porcine astrovirus, (Astroviridae), Porcine circovirus, Circoviridae, Porcine enteric calicivirus, (Caliciviridae), Porcine enterovirus, (Picornaviridae), Porcine epidemic diarrhea virus, (Coronaviridae), Porcine hemagglutinating encephalomyelitis virus, (Coronaviridae), Porcine parvovirus, (Parvoviridae), Porcine respiratory and reproductive syndrome, (Arterivirus), Porcine rubulavirus, (Paramyxoviridae), Porcine transmissible gastroenteritis virus, (Coronaviridae), Porcine type C oncovirus, (Retroviridae), Porton virus, (Rhabdoviridae), Potosi virus, (Bunyaviridae), Powassan virus, (Flaviviridae), Precarious Point virus, (Bunyaviridae), Pretoria virus, (Bunyaviridae), Primate calicivirus, (Caliciviridae), Prospect Hill virus, (Bunyaviridae), Pseudaletia includens densovirus, (Parvoviridae), Pseudocowpox virus, (Poxviridae), Pseudolumpy skin disease virus, (Herpesviridae), Pseudorabies virus, (Herpesviridae), Psittacid herpesvirus, (Herpesviridae), Psittacinepox virus, (Poxviridae), Puchong virus, (Rhabdoviridae), Pueblo Viejo virus, (Bunyaviridae), Puffin Island virus, (Bunyaviridae), Punta Salinas virus, (Bunyaviridae), Punta Toro virus, (Bunyaviridae), Purus virus, (Reoviridae), Puumala virus, (Bunyaviridae), Qalyub virus, (Bunyaviridae), Quailpox virus, (Poxviridae), Quokkapox virus, (Poxviridae), Rabbit coronavirus, (Coronaviridae), Rabbit fibroma virus, (Poxviridae), Rabbit hemorrhagic disease virus, (Caliciviridae), Rabbit kidney vacuolating virus, (Papovaviridae), Rabbit oral papillomavirus, (Papovaviridae), Rabbitpox virus, (Poxviridae), Rabies virus, (Rhabdoviridae), Raccoon parvovirus, (Parvoviridae), Raccoonpox virus, (Poxviridae), Radi virus, (Rhabdoviridae), Rangifer tarandus herpesvirus, (Herpesviridae), Ranid herpesvirus, (Herpesviridae), Raphanus virus, (Rhabdoviridae), Rat coronavirus, (Coronaviridae), Rat cytomegalovirus, (Herpesviridae), Rat virus, R, (Parvoviridae), Raza virus, (Bunyaviridae), Razdan virus, (Bunyaviridae), Red deer herpesvirus, (Herpesviridae), Red kangaroopox virus, (Poxviridae), Reed Ranch virus, (Rhabdoviridae), herpesvirus, (Herpesviridae), Reindeer papillomavirus, (Papovaviridae), Reptile calicivirus, (Caliciviridae), Resistencia virus, (Bunyaviridae), Restan virus, (Bunyaviridae), Reticuloendotheliosis virus, (Retroviridae), Rhesus HHV-like virus, (Herpesviridae), Rhesus leukocyte associated herpesvirus strain, (Herpesviridae), Rhesus monkey cytomegalovirus, (Herpesviridae), Rhesus monkey papillomavirus, (Papovaviridae), Rheumatoid arthritis virus, (Parvoviridae), Rift Valley fever virus, (Bunyaviridae), Rinderpest virus, (Paramyxoviridae), Rio Bravo virus, (Flaviviridae), Rio Grande virus, (Bunyaviridae), RML virus, (Bunyaviridae), Rochambeau virus, (Rhabdoviridae), Rocio virus, (Flaviviridae), Ross River virus, (Togaviridae), Rost Islands virus, (Reoviridae), Rous sarcoma virus, (Retroviridae), Royal farm virus, (Flaviuiridae), RT parvovirus, (Parvoviridae), Rubella virus, (Togaviridae), Russian spring summer encephalitis virus, (Flaviviridae), S-virus, (Reoviridae), SA virus, (Herpesviridae), Sabio virus, (Arenaviridae), Sabo virus, (Bunyaviridae), Saboya virus, (Flaviviridae), Sacbrood virus, (Picornaviridae), Sagiyama virus, (Togaviridae), Saimiriine herpesvirus, (Herpesviridae), SaintAbb's Head virus, (Reoviridae), Saint-Floris virus, (Bunyaviridae), Sakhalin virus, (Bunyaviridae), Sal Vieja virus, (Flaviviridae), Salanga virus, (Bunyaviridae), Salangapox virus, (Poxviridae), Salehabad virus, (Bunyaviridae), Salmonid herpesvirus, (Herpesviridae), Salmonis virus, (Rhabdoviridae), Sambucus vein clearing virus, (Rhabdoviridae), SanAngelo virus, (Bunyaviridae), San Juan virus, (Bunyaviridae), San Miguel sealion virus, (Caliciviridae), San Perlita virus, (Flaviviridae), Sand rat nuclear inclusion agents, (Herpesviridae), Sandfly fever Naples virus, (Bunyaviridae), Sandfly fever Sicilian virus, (Bunyaviridae), Sandjimba virus, (Rhabdoviridae), Sango virus, (Bunyaviridae), Santa Rosa virus, (Bunyaviridae), Santarem virus, (Bunyaviridae), Sapphire II virus, (Bunyaviridae), Saraca virus, (Reoviridae), Sarracenia purpurea virus, (Rhabdoviridae), Sathuperi virus, (Bunyaviridae), Saumarez Reef virus, (Flaviviridae), Sawgrass virus, (Rhabdoviridae), Schistocerca gregaria entomopoxvirus, (Poxviridae), Sciurid herpesvirus, (Herpesviridae), Sciurid herpesvirus, (Herpesviridae), Sealpox virus, (Poxviridae), Seletar virus, (Reoviridae) Semliki Forest virus, (Togaviridae), Sena Madureira virus, (Rhabdoviridae), Sendai virus, (Paramyxoviridae), Seoul Virus, (Bunyaviridae), Sepik virus, (Flaviviridae), Serra do Navio virus, (Bunyaviridae), Shamonda virus, (Bunyaviridae), Shark River virus, (Bunyaviridae), Sheep associated malignant catarrhal fever of, (Herpesviridae), Sheep papillomavirus, (Papovaviridae), Sheep pulmonary adenomatosis associated herpesvirus, (Herpesviridae), Sheeppox virus, (Poxviridae), Shiant Islands virus, (Reoviridae), Shokwe virus, (Bunyaviridae), Shope fibroma virus, (Poxviridae), Shuni virus, (Bunyaviridae), Sibine fusca densovirus, (Parvoviridae), Sigma virus, (Rhabdoviridae), Sikte water-borne virus, (Tombusviridae), Silverwater virus, (Bunyaviridae), virus, (Bunyaviridae), Simian adenoviruses, (Adenoviridae), Simian agent virus, (Papovaviridae), Simian enterovirus, (Picornaviridae), Simian foamy virus, (Retroviridae), Simian hemorrhagic fever virus, (Arterivirus), Simian hepatitis A virus, (Picornaviridae), Simian immunodeficiency virus, (Retroviridae), Simian parainfluenza virus, (Paramyxoviridae), Simian rotavirus SA, (Reoviridae), Simian sarcoma virus, (Retroviridae), Simian T-lymphotropic virus, (Retroviridae), Simian type D virus, (Retroviridae), Simian vancella herpesvirus, (Herpesviridae), Simian virus, (Papovaviridae), Simulium vittatum densovirus, (Parvoviridae), Sindbis virus, (Togaviridae), Sixgun city virus, (Reoviridae), Skunkpox virus, (Poxviridae), Smelt reovirus, (Reoviridae), Snakehead rhabdovirus, (Rhabdoviridae), Snowshoe hare virus, (Bunyaviridae), Snyder-Theilen feline sarcoma virus, (Retroviridae), Sofyn virus, (Flaviviridae), Sokoluk virus, (Flaviviridae), Soldado virus, (Bunyaviridae), Somerville virus, (Reoviridae), Sparrowpox virus, (Poxviridae), Spectacled caimanpox virus, (Poxviridae), SPH virus, (Arenaviridae), Sphenicid herpesvirus, (Herpesviridae), Spider monkey herpesvirus, (Herpesviridae), Spondweni virus, (Flaviviridae), Spring viremia of carp virus, (Rhabdoviridae), Squirrel fibroma virus, (Poxviridae), Squirrel monkey herpesvirus, (Herpesviridae), Squirrel monkey retrovirus, (Retroviridae), SR-virus, (Bunyaviridae), Sripur virus, (Rhabdoviridae), StAbbs Head virus, (Bunyaviridae), St. Louis encephalitis virus, (Flaviviridae), Starlingpox virus, (Poxviridae), Stratford virus, (Flaviviridae), Strigid herpesvirus, (Herpesviridae), Striped bass reovirus, (Reoviridae), Striped Jack nervous necrosis virus, (Nodaviridae), Stump-tailed macaque virus, (Papovaviridae), Suid herpesvirus, (Herpesviridae), Sunday Canyon virus, (Bunyaviridae), Sweetwater Branch virus, (Rhabdoviridae), Swine cytomegalovirus, (Herpesviridae), Swine infertility and respiratory syndrome virus, (Arterivirus), Swinepox virus, (Poxviridae), Tacaiuma virus, (Bunyaviridae), Tacaribe virus, (Arenaviridae), Taggart virus, (Bunyaviridae), Tahyna virus, (Bunyaviridae), Tai virus, (Bunyaviridae), Taiassui virus, (Bunyaviridae), Tamana bat virus, (Flaviviridae), Tamdy virus, (Bunyaviridae), Tamiami virus, (Arenaviridae), Tanapox virus, (Poxviridae), Tanga virus, (Bunyaviridae), Tanjong Rabok virus, (Bunyaviridae), Taro bacilliform virus, (Badnavirus), Tataguine virus, (Bunyaviridae), Taterapox virus, (Poxviridae), Tehran virus, (Bunyaviridae), Telok Forest virus, (Bunyaviridae), Tembe virus, (Reoviridae), Tembusu virus, (Flaviviridae), Tench reovirus, (Reoviridae), Tensaw virus, (Bunyaviridae), Tephrosia symptomless virus, (Tombusviridae), Termeil virus, (Bunyaviridae), Tete virus, (Bunyaviridae), Thailand virus, (Bunyaviridae), Theiler's murine encephalomyelitis virus, (Picornaviridae), Thermoproteus virus, Lipothrixviridae, Thiafora virus, (Bunyaviridae), Thimiri virus, (Bunyaviridae), Thogoto virus, (Orthomyxoviridae), Thormodseyjarklettur virus, (Reoviridae), Thottapalayam virus, (Bunyaviridae), Tibrogargan virus, (Rhabdoviridae), Tick-borne encephalitis virus, (Flaviviridae), Tillamook virus, (Bunyaviridae), Tilligerry virus, (Reoviridae), Timbo virus, (Rhabdoviridae), Tilmboteua virus, (Bunyaviridae), Tilmaroo virus, (Bunyaviridae), Tindholmur virus, (Reoviridae), Tlacotalpan virus, (Bunyaviridae), Toscana virus, (Bunyaviridae), Tradescantia/Zebrina virus, Potyviridae, Trager duck spleen necrosis virus, (Retroviridae), Tree shrew adenovirus, (Adenoviridae), Tree shrew herpesvims, (Herpesviridae), Triatoma virus, (Picornaviridae), Tribec virus, (Reoviridae), Trivittatus virus, (Bunyaviridae), Trombetas virus, (Bunyaviridae), Trubanarnan virus, (Bunyaviridae), Tsuruse virus, (Bunyaviridae), Tucunduba virus, (Bunyaviridae), Tumor virus X, (Parvoviridae), Tupaia virus, (Rhabdoviridae), Tupaiid herpesvirus, (Herpesviridae), Turbot herpesvirus, (Herpesviridae), Turbot reovirus, (Reoviridae), Turkey adenoviruses, (Adenoviridae), Turkey coronavirus, (Coronaviridae), Turkey herpesvirus, (Herpesviridae), Turkey rhinotracheitis virus, (Paramyxoviridae), Turkeypox virus, (Poxviridae), Turlock virus, (Bunyaviridae), Turuna virus, (Bunyaviridae), Tyuleniy virus, (Flaviviridae) Uasin Gishu disease virus, (Poxviridae), Uganda S virus, (Flaviviridae), Ulcerative disease rhabdovirus, (Rhabdoviridae), Umatilla virus, (Reoviridae), Umbre virus, (Bunyaviridae), Una virus, (Togaviridae), Upolu virus, (Bunyaviridae), UR sarcoma virus, (Retroviridae), Urucuri virus, (Bunyaviridae), Usutu virus, (Flaviviridae), Uting a virus, (Bunyaviridae), Utive virus, (Bunyaviridae), Uukuniemi virus, (Bunyaviridae) Vaccinia subspecies, (Poxviridae), Vaccinia virus, (Poxviridae), Vaeroy virus, (Reoviridae), Varicella-zoster virus, (Herpesviridae), Variola virus, (Poxviridae), Vellore virus, (Reoviridae), Venezuelan equine encephalitis virus, (Togaviridae), Vesicular exanthema of swine virus, (Caliciviridae), Vesicular stomatitis Alagoas virus, Rhabdoviridae, Vesicular stomatitis Indiana virus, (Rhabdoviridae), Vesicular stomatitis New Jersey virus, (Rhabdoviridae), Vilyuisk virus, (Picornaviridae), Vinces virus, (Bunyaviridae), Viper retrovirus, (Retroviridae), Viral hemorrhagic septicemia virus, (Rhabdoviridae), Virgin River virus, (Bunyaviridae), Virus III, (Herpesviridae), Visna/maedi virus, (Retroviridae), Volepoxvirus, (Poxviridae), Wad Medani virus, (Reoviridae), Wallal virus, (Reoviridae), Walleye epidermal hyperplasia, (Herpesviridae), Wanowrie virus, (Bunyaviridae), Warrego virus, (Reoviridae), Weddel water-borne virus, Tombusviridae, Weldona virus, (Bunyaviridae), Wesselsbron virus, (Flaviviridae), West Nile virus, (Flaviviridae), Western equine encephalitis virus, (Togaviridae), Wexford virus, (Reoviridae), Whataroa virus, (Togaviridae), Wildbeest herpesvirus, (Herpesviridae), Witwatersrand virus, (Bunyaviridae), Wongal virus, (Bunyaviridae), Wongorr virus, (Reoviridae), Woodchuck hepatitis B virus, (Hepadnaviridae), Woodchuck herpesvirus marmota, (Herpesviridae), Woolly monkey sarcoma virus, (Retroviridae), Wound tumor virus, (Reoviridae), WVU virus, (Reoviridae), WW virus, (Reoviridae), Wyeomyia virus, (Bunyaviridae), Xiburema virus, (Rhabdoviridae), Xingu virus, (Bunyaviridae), Y sarcoma virus, (Retroviridae), Yaba monkey tumor virus, (Poxviridae), Yaba-virus, (Bunyaviridae), Yaba-virus, (Bunyaviridae), Yacaaba virus, (Bunyaviridae), Yaounde virus, (Flaviviridae), Yaquina Head virus, (Reoviridae), Yata virus, (Rhabdoviridae), Yellow fever virus, (Flaviviridae), Yogue virus, (Bunyaviridae), Yokapox virus, (Poxviridae), Yokase virus, (Flaviviridae), Yucca baciliform virus, Badnavirus, Yug Bogdanovac virus, (Rhabdoviridae), Zaliv Terpeniya virus, (Bunyaviridae), Zea mays virus, (Rhabdoviridae), Zegla virus, (Bunyaviridae), Zika virus, (Flaviviridae), and Zirqa virus, (Bunyaviridae).

### Pathogenic Infectious Agents

Pathogenic infectious agents which can be removed, prevented, and/or treated by the air purification system and methods of the disclosure include, for example: an intracellular pathogen, i.e. a pathogen capable of growing and reproducing inside the cells of a host. Bacterial examples which may be prevented and/or treated by the compostions and methods of the disclosure include but are not limited to Francisella tularensis, Listeria monocytogenes, Salmonella, Brucella, Legionella, Mycobacterium, Nocardia, Rhodococcus equi, Yersinia, Neisseria meningitidis, Chlamydia, Rickettsia, Coxiella, Mycobacterium, such as Mycobacterium leprae and Treponema pallidum. Fungal examples include but are not limited to Histoplasma capsulatum, Cryptococcus neoformans and Pneumocystis jirovecii. Examples of protozoa include but are not limited to Apicomplexans (e.g. Plasmodium spp., Toxoplasma gondii and Cryptosporidium parvum) and Trypanosomatids (e.g. Leishmania spp. and Trypanosoma cruzi).

The following is an exemplary but non-limiting discussion of various disease agents that could be the subject of prevention and/or treatment in accordance with the present disclosure.

Bacterial Pathogenic Agents—The air purification system and methods of the disclosure can remove, prevent, and/or treat Bacterial Pathogenic Agents. There are hundreds of bacterial pathogens in both the Gram-positive and Gram-negative families that cause significant illness and mortality around the word, despite decades of effort developing antibiotic agents. Antibiotic resistance is a growing problem in bacterial disease. Bacterial pathogens can be removed, prevented, and/or treated by the air purification system and methods of the disclosure.

One of the bacterial diseases with highest disease burden is tuberculosis, caused by the bacterium Mycobacterium tuberculosis, which kills about 2 million people a year, mostly in sub-Saharan Africa. Pathogenic bacteria contribute to other globally important diseases, such as pneumonia, which can be caused by bacteria such as Streptococcus and Pseudomonas, and food borne illnesses, which can be caused by bacteria such as Shigella, Campylobacter, and Salmonella. Pathogenic bacteria also cause infections such as tetanus, typhoid fever, diphtheria, syphilis, and leprosy.

Conditionally pathogenic bacteria are only pathogenic under certain conditions, such as a wound facilitates entry of bacteria into the blood, or a decrease in immune function. For example, Staphylococcus or Streptococcus are also part of the normal human flora and usually exist on the skin or in the nose without causing disease, but can potentially cause skin infections, pneumonia, meningitis, and even overwhelming sepsis, a systemic inflammatory response producing shock, massive vasodilation and death. Some species of bacteria, such as Pseudomonas aeruginosa, Burkholderia cenocepacia, and Mycobacterium avium, are opportunistic pathogens and cause disease mainly in people suffering from immunosuppression or cystic fibrosis.

Other bacteria invariably cause disease in humans, such as obligate intracellular parasites (e.g., Chlamydophila, Ehrlichia, Rickettsia) that are capable of growing and reproducing only within the cells of other organisms. Still, infections with intracellular bacteria may be asymptomatic, such as during the incubation period. An example of intracellular bacteria is Rickettsia. One species of Rickettsia causes typhus, while another causes Rocky Mountain spotted fever. Chlamydia, another phylum of obligate intracellular parasites, contains species that can cause pneumonia or urinary tract infection and may be involved in coronary heart disease. Mycobacterium, Brucella, Francisella, Legionella, and Listeria can exist intracellular, though they are facultative (not obligate) intracellular parasites.

Gram-positive bacteria include Staphylococcus aureus; Staphylococcus epidermidis; Staphylococcus saprophyticus; Streptococcus pyogenes (Lancefield group A, beta-hemolytic); Streptococcus agalactiae (Lancefield group B, beta-hemolytic); Streptococcus viridans group (most are alpha-hemolytic) including, for example, the Mitus group (S. mitus, S. sanguis, S. parasanguis, S. gordonii, S. crista, S. infantis, S. oralis, S. peroris), the Salivarius group (S. salivarius, S. vestibularis, S. thermophilus), the Mutans group (S. mutans, S. sobrinus, S. criceti, S. rattus, S. downei, S. macacae), and the Anginosus group (S. anginosus, S. constellatus, S. intermedius); Streptococcus, e.g., S. bovis, S. equinus (Lancefield group D, alpha-hemolytic); Streptococcuspneumoniae (no Lancefield antigen; alpha-hemolytic); Peptostreptococcus and Peptococcus; Entercoccus faecalis; Enterococcus faeccium; Cornybacterium diphtheria; Bacillus anthracis; Bacillus cereus; Clostridium C. botulinum (more rarely, C. baratii and C. butyricum); Clostridium tetani; Clostridium perfringens; Clostridium difficile; Clostridium sordellii; Listeria monocytogenes; Actinomyces israelii; Nocardia asteroids; and Streptomyces.

Gram-negative bacteria include Neisseria meningitides; Neisseria gonorrhoeae; Moraxella (subgenera Branhamella) catarrhalis; Kingella (most commonly kingae); Acinetobacter baumannii, Oligella ureolytica; Oligella urethralis; Escherichia coli; Shigella (S. dysenteriae, S. flexneri, S. boydii, S. sonnei); Salmonella non typhoidal, including S. enterica serotype enteritidis, S. enterica serotype typhimurium, S. enterica serotype Choleraesuis, S. bongori, Salmonella S. enterica serotype Typhi; Yersinia enterocolitica, Klebsiella pneumoniae; Proteus mirabilis; Enterobacter; Cronobacter (formerly called Enterobacter sakazakii); Serratia; Edwardsiella; Citrobacter; Hafnia; Providencia; Vibrio cholera; Vibrio parahemolyticus; Campylobacter; Helicobacter (formerly called Campylobacter) pylori, Pseudomonas aeruginosa; Burkholderia cepacia; Burkholderia mallei; Burkholderia pseudomallei; Stenotrophomonas maltophilia; Bacteroides fragilis, Bacteroides melaninogenicus; Fusobacterium; Haemophilus influenza; Haemophilus ducreyi; Gardnerella (formerly called Haemophilus) vaginalis; Bordetella pertussis; Legionella; Yersinia pestis; Francisella tularensis; Brucella B. melitensis (infects sheep/goats); B. abortus (abortions in cows); B. suis (pigs); B. canis (dogs); B. maris (marine animals); Pasteurella multocida; Streptobacillus moniliformis; Spirillum minus; Treponema pallidum; Treponema pallidum subspecies pertenue; Treponema pallidum subspecies endemicum; Treponema pallidum subspecies carateum; Borrelia burgdorferi; Borrelia; Leptospira; Chlamydia trachomatis; Chlamydia pneumonia; Chlamydia psittaci; Rickettsiae rickettsia; Rickettsiae akari; Rickettsiae prowazekii; Rickettsiae typhi; Rickettsiae tsutsugamushi; Rickettsiae parkeri; Rickettsiae africae; Rickettsia conorii; Rickettsia australis; Rickettsia siberica; Rickettsia japonica; Bartonella Quintana; Bartonella henselae; Bartonella bacilliformis; Coxiella burnetii; Ehrlichia; Anaplasma phagocytophilum; Neorickettsia; Orientia; Klebsiella granulomatis (formerly called Calymmatobacterium granulomatis); and Capnocytophaga.

Other bacteria include Mycobacterium tuberculosis; Mycobacterium bovis; Mycobacterium leprae; Mycobacterium avium-intracellulare or avium complex (MAI or MAC); Mycobacterium ulcerans; Mycobacterium kansasii; Mycobacterium marinum; Mycobacterium scrofulaceum; Mycobacterium fortuitum; Mycobacterium chelonei; Mycobacterium abscessus; Mycoplasma pneumonia; and Ureaplasma urealyticum.

Fungal Pathogens—The air purification system and methods of the disclosure can remove, prevent, and/or treat fungal pathogenic agents that cause disease in humans or other organisms. The pathogenic fungi which may be removed, prevented and/or treated by the system and methods of the disclosure include but are not limited to the following Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; and Exserohilum.

Candida species are important human pathogens that are best known for causing opportunist infections in immunocompromised hosts (e.g., transplant patients, AIDS sufferers, and cancer patients). Infections are difficult to treat and can be very serious. Aspergillus can and does cause disease in three major ways: through the production of mycotoxins; through induction of allergenic responses; and through localized or systemic infections. With the latter two categories, the immune status of the host is pivotal. The most common pathogenic species are Aspergillus fumigatus and Aspergillus flavus. Cryptococcus neoformans can cause a severe form of meningitis and meningo-encephalitis in patients with HIV infection and AIDS. The majority of Cryptococcus species lives in the soil and do not cause disease in humans. Cryptococcus laurentii and Cryptococcus albidus have been known to occasionally cause moderate-to-severe disease in human patients with compromised immunity. Cryptococcus gattii is endemic to tropical parts of the continent of Africa and Australia and can cause disease in non-immunocompromised people. Histoplasma capsulatum can cause histoplasmosis in humans, dogs and cats. Pneumocystis jirovecii (or Pneumocystis carinii) can cause a form of pneumonia in people with weakened immune systems, such as premature children, the elderly, transplant patients and AIDS patients. Stachybotrys chartarum or “black mold” can cause respiratory damage and severe headaches. It frequently occurs in houses in regions that are chronically damp.

Parasitic Agents—The air purification systems and methods of the disclosure can remove, prevent, and/or treat parasitic agents. Parasites present a major health issue, particularly in under-developed countries around the world. Significant pathogenic parasites which may be removed, prevented and/or treated by the systems and methods of the disclosure include worms (roundworms, flatworms) and protozoa, Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, e.g., C. parvum, C. hominis; Cystoisospora species (formerly called Isospora species), e.g., C. belli; Naegleria fowleri; Acanthamoeba species; Sappinia diploidea; Sappinia pedata; Balamuthia mandrillaris; Pneumocystis jiroveci (formerly called Pneumocystis carinii); Plasmodium knowlesi; Babesia microti; Babesia divergens; Babesia duncani; Babesia (no species name yet but designated MO-1); Trypanosoma brucei rhodesiense; Trypanosoma brucei gambiense; Balantidium coli; Dientamoeba fragilis; Phylum: Microsporidia; Sarcocystis; Baylisascaris; Necator americanus; Ancylostoma duodenale; Strongloides stercoralis; Trichinella pseudospiralis; Trichinella nelsoni; Trichinella britovi; Trichinella nativa; Trichuris trichiura; Enterobius vermicularis; Anisakis simplex; Pseudoterranova decipiens; Trichostrongylus; Oesophagostomum, e.g., O. bifurcom; Angiostrongylus; Capillaria; Dirofilaria; Loa boa; Onchocerca volvulus; Wuchereria bancrofti; Brugia malayi; Brugia timori; Mansonella, M. perstans; M. streptocerca; M. ozzardi; Dracunculus mediensis; Cutaneous larva migrans (commonly Ancylostoma braziliense=dog hookworm; also A. caninum, A. ceylanicum, and Uncinaria stenocephala); Visceral larva migrans (most commonly Toxocara canis=dog roundworm, less commonly Toxocara cati=cat roundworm, Baylisascaris procyonis=raccoon roundworm) or ocular larva migrans or neural larva migrans (B. procyonis); Gnathostoma G. spinigerum and G. hispidum; Dicrocoelium dendriticum; Echinostoma, e.g., E. hortense, E. macrorchis, E. revolutum, E. ilocanu, and E. perfoliatum; Thelazia; Shistosoma japonicum; Shistosoma mansoni; Shistosoma haematobium; Shistosoma intercalatum; Shistosoma mekongi; Austrobilharzia variglandis and other schistosomes; Taenia solium; Taenia saginata; Taenia multiceps; Taenia serialis; Taenia asiatica; Diphyllobothrium latum; Hymenolepsis nana; Echinoccoccus; Paragonimus; Clonorchis sinensis; Dipylidium caninum; Fasciola, F. hepatica; F. gigantica; Fasciolopsis buski; Heterophyes heterophyes; Hymenolepsis, H. nana, H. dimnuta; Opisthorchis; Bertiella, e.g., B. studeri and B. mucronata; Macracanthorhynchus hirudinaceous; Moniliformis moniliformis; Bolbosoma species; Metagonimus yokogawai; Dioctophyme renale; Mesocestoides, e.g., M. lineatus and M. variabilis; Philophthalmus, e.g., P. lacrymosus, P. gralli, P. palpebrarum; Spirometra, e.g., S. mansoni, S. ranarum, S. mansonoides, S. erinacei; and Sparganum proliferum.

### Chemical Contaminants

Chemical contaminants can be removed, prevented, and/or treated by the air purification systems and methods of the disclosure. Tobacco smoke, engine exhaust, and similar allergens and odors or odor-causing agents can be abated by system and methods of the disclosure, as can VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like. VOCs include organic solvents, certain paint additives, aerosol spray can propellants, fuels (such as gasoline and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone. Odors and odor-causing substances that can be abated include skunk odors, urine, pet odors, and the like.

### Particulate Contaminants

Particulate contaminants can be removed, prevented, and/or treated by the air purification systems and methods of the disclosure. Common household molds that can be remediated by the system and methods of the disclosure include, but are not limited to Acremonium; Alternaria; Aspergillus fumigatus; Aspergillus niger; Aspergillus species Var. 1; Aspergillus species Var. 2; Aureobasidium; Bipolaris; Chaetomium; Cladosporium; Curvularia; Epicoccum; Fusarium; Geotrichum; Memnoniella; Mucor; Mycelia sterilia; Nigrospora; Paecilomyces; Penicillium species Var. 1; Penicillium species Var. 2; Pithomyces; Rhizopus; Sporothrix; Sporotrichum; Stachybotrys; Syncephalastrum; Trichoderma; and Yeast. Molds need high humidity levels and a surface on which to grow. Common areas for mold growth are garbage containers, food storage areas, upholstery, and wallpaper. Molds also commonly grow in damp areas such as basements, shower curtains, window moldings, and window air conditioners.

Indoor allergens that can be remediated by system and methods of the disclosure include dust mite feces. Dust mite feces are the major source of allergic reaction to household dust. The mites thrive on shed human skin and are most commonly found in bedrooms, where skin cells are abundant. Preventive measures include frequently laundering bed linens in hot water and removing carpets from the room. In some cases, homeowners might have to encase the bed mattress, box springs, and pillows in vinyl covers. Other allergens of animal origin include skin scales shed from humans and animals. Commonly called dander, these are another major allergen. Dander from such animals as cats, dogs, horses, and cows can infest a home even if the animal has never been inside. Rodent urine from mice, rats, and guinea pigs is another allergen. Cockroach-derived allergens come from the insect's discarded skins. As the skins disintegrate over time, they become airborne and are inhaled.

### Health Care Facility

The air purification technology, systems, and methods of the disclosure will reduce the levels of airborne biological, chemical, and particulate contamination and thus improve outcomes in all areas of, for example, basic, applied, commercial, industrial, biological, and chemical research and manufacturing, as well as health care facilities, and long-term care facilities, as set forth herein.

The Pennsylvania Healthcare Cost Containment Council (PHC4) Report indicated that hospitals and Medicare spent over $3B and $400M, respectively, towards patients affected by HAIs in the State of Pennsylvania during the reporting year of 2011. HAI infections are reported by the hospitals to the Centers for Disease Control and Prevention's (CDC's) National Healthcare Safety Network (NHSN), which is now the primary data collection tool used for HAI reporting by more than 5,000 hospitals across the country. The information submitted to NHSN from Pennsylvania hospitals is provided to the Pennsylvania Department of Health (PADOH) in order to be compiled, analyzed and published on an annual basis.

HAIs are usually classified into 13 major categories. The 3 most significant categories include Surgical Site Infections (SSI) (26.9%), Urinary Tract Infections (UTI) (22.3%), and Gastrointestinal Infections (17.4%). There are additional HAIs that are classified into the category, “other.” The 2011 PADOH report indicated that of the 13 categories analyzed, the HAI rates increased in 3 categories, demonstrated no change in 5 of the categories, and showed a reduction in HAI rates in 5 of the 13 categories when compared to the 2010 PADOH report data. Continued focus on improvements in infection control strategies and protocol implementation are necessary to reverse the reported trend of a static to a decline in the effectiveness of current infection control strategies.

Historically, it has been understood that the source of the pathogens responsible for HAIs are associated with the patient and surface areas. Infection control protocols, patient preparation protocols and hand-washing protocols have been implemented in most hospitals and have been helpful in reducing overall HAI rates. Recent literature has suggested that significant sources of pathogens responsible for HAIs are airborne. The 2011 PADOH report indicated that SSIs represented 26.9% of the HAIs when analyzed by clinical procedure. Analysis of the pathogens responsible for the SSIs demonstrated that 69% of these pathogens are potentially airborne. There are multiple national standards for air filtration in hospital settings that vary dependent upon the protocols specific to the hospital. The air serving the clinical areas is often a combination of outside air and recirculated air. Outside source air will contribute a variable and dynamic mix of microbials and the contamination level is specific to the location of the hospital. An urban or high-density area will exert a greater biological and chemical contaminant load on the air serving the clinical area. Recirculated air can often carry greater loads of biological and chemical contaminants than the outside source air. Studies have demonstrated that both properly gowned healthcare workers and patients shed approximately 3,000-50,000 microorganisms per minute or 0.12 colony forming units (cfu) per cubic meter per minute. Approximately 10% of the shed microorganisms can be infectious. At this rate of microbial contribution, the recommended level of microbial load within the typical operating room (OR) would be exceeded within 10 minutes. Airborne microbials constitute a large source of pathogens responsible for SSIs. The high number of air changes per hour, laminar flow, and OR personnel movement and traffic within the OR contribute to the maintenance of airborne pathogens. Many pathogens can remain airborne and viable for weeks at a time. It is critical to reduce the constant and high level of microbial contribution within the outside and recirculated air and remediate the threatening biologicals.

In an effort to filter the air, most hospitals employ HEPA filters to reduce levels of biological or microbial contamination. Attempts to control airborne infection rates have included the use of multiple layers of HEPA filtration and the use of UVGI. Several studies were able to document a marked reduction in infection cases following the introduction of UVGI. The system and methods of the disclosure employs the use of UVGI technology. However, a ten-year study of the effectiveness of UVGI on the irradiation of biologicals and the concomitant impact on clinical outcome data led to the development of an advanced and more effective UVGI technology.

The health care facility may be one of a medical unit, a surgical unit, a critical care unit, an intensive care unit, an emergency care unit, a pediatric unit, an emergency unit, an outpatient unit, a specialty care unit, a dermatology unit, an endocrinology unit, a gastroenterology, an internal medicine unit, an oncology unit, a neurology unit, an orthopedic unit, an ophthalmic unit, an ear nose and throat unit, a neonatal unit, an obstetrics and gynecology unit, a cardiac unit, a psychiatric unit, a post-operative recovery unit, a radiology unit, a plastic surgery unit and an urology unit. The health care facility may be one or more of a bed, a room, a ward, a unit, a floor, a facility and a hospital group.

The health care facility may be, for example, a Hospital, a non-teaching Hospital, a teaching Hospital (affiliated with medical school), Rehabilitation Facility, Long Term Care Facility, Free Standing Surgical Center, Medical/physician office Dental office, Veterinarian Office, Adult Critical Care Units, Burn Critical Care, Medical Cardiac Critical Care, Medical Critical Care, Medical/Surgical Critical Care, Neurologic Critical Care, Neurosurgical Critical Care, Prenatal Critical Care, Respiratory Critical Care, Surgical Cardiothoracic Critical Care, Surgical Critical Care, Trauma Critical Care, Pediatric Critical Care Units, Pediatric Burn Critical Care, Pediatric Cardiothoracic Critical Care, Pediatric Medical Critical Care, Pediatric Medical/Surgical Critical Care, Pediatric Neurology Critical Care, Pediatric Neurosurgical Critical Care, Pediatric Respiratory Critical Care, Pediatric Surgical Critical Care, Pediatric Trauma Critical Care, Neonatal Units, Well Baby Nursery (Level I), Step down Neonatal ICU (Level II) Neonatal Critical Care (Level II/III) Neonatal Critical Care (Level III), Inpatient Specialty Care Areas, Long Term Acute Care (LTAC) Bone Marrow Transplant Specialty Care Area, Acute Dialysis Unit, Hematology/Oncology SCA, Solid Organ Transplant SCA, Pediatric Bone Marrow Transplant SCA, Pediatric Dialysis SCA, Pediatric Hematology/Oncology SCA, Pediatric Long-Term Acute Care, Pediatric Solid Organ Transplant SCA, Inpatient Adult Wards, Antenatal Care Ward, Burn Ward, Behavioral Health/Psych Ward, Ear/Nose/Throat Ward, Gastrointestinal Ward, Gerontology Ward, Genitourinary Ward, Gynecology Ward, Jail Unit, Labor and Delivery Ward, Labor, Delivery, Recovery, Postpartum Room (LDRP), Medical Ward, Medical/Surgical Ward, Mixed Acuity Ward, Mixed Age, Mixed Acuity Ward, Neurology Ward, Neurosurgical Ward, Ophthalmology Ward, Orthopedic Trauma Ward, Orthopedic Ward, Plastic Surgery Ward, Postpartum Ward, Pulmonary Ward, Rehabilitation Ward, School Infirmary, Surgical Ward, Stroke (Acute) Unit, Telemetry Unit, Vascular Surgery Ward, Inpatient Pediatric Wards, Adolescent Behavioral Health, Pediatric Burn Ward, Pediatric Behavioral Health, Pediatric Ear, Nose, Throat, Pediatric Genitourinary, Medical Pediatric WardPediatric Med/Surg Ward, Pediatric Mixed Acuity (if patients are of mixed age, use Mixed Age found in Inpatient Adult Wards) Pediatric Neurology Ward, Pediatric Neurosurgical Ward. Pediatric Orthopedic Ward, Pediatric Rehabilitation Ward, Pediatric Surgical WardStep Down Units, Step Down Unit, Pediatric Step Down Unit, Operating Rooms, Cardiac Catheterization Room/Suite, Cesarean Section Room/Suite, Interventional Radiology, Operating Room/Suite, Post Anesthesia Care Unit/Recovery Room, Long Term Care, Inpatient Hospice, Long Term Care Unit, Long Term Care Alzheimer's Unit, Long Term Care Behavioral Health/Psych Unit, Ventilator Dependent Unit, Long Term Care Rehabilitation Unit; Laboratory Identified Event (LabID) Only Facility-wide Inpatient, Facility-wide Outpatient, Miscellaneous Areas—All Inpatient Beds Combined, Float, Sleep Studies (for in and out patients) Pulmonary Function Testing, Transport Service, Treatment Room, Outpatient Locations, Acute Care Settings24-Hour Observation Area, Ambulatory Surgery Center, Facility-wide Outpatient, Mobile Emergency Services/EMS, Outpatient Emergency Department, Outpatient Pediatric Surgery Center, Outpatient Plastic Surgery Center, Outpatient Surgery Recovery Room/Post Anesthesia Care Unit, Pediatric Emergency Department, Therapeutic Apheresis Unit, Urgent Care Center, Clinic (Nonacute) Settings, Allergy Clinic, Behavioral Health Clinic, Blood Collection Center, Cardiac Rehabilitation Center, Cardiology Clinic, Continence Clinic, Dermatology Clinic, Diabetes/Endocrinology Clinic, Ear, Nose, Throat Clinic, Family Medicine Clinic, Genetics Clinic, Gynecology Clinic, Holistic Medicine Center, Hyperbaric Oxygen Center, Infusion Center, Neurology Clinic, Occupational Health Clinic, Occupational Therapy Clinic, Ophthalmology Clinic, Orthopedic Clinic, Ostomy Clinic, Outpatient Dental Clinic, Outpatient GI Clinic, Outpatient Hematology/Oncology Clinic, Outpatient Hemodialysis Clinic, Outpatient HIV Clinic, Outpatient Medical Clinic, Outpatient Rehabilitation Clinic, Pain Clinic, Pediatric Behavioral Health Clinic, Pediatric Cardiology Center, Pediatric Clinic, Pediatric Dental Clinic, Pediatric Dermatology Clinic, Pediatric Diabetes/Endocrinology Clinic, Pediatric Gastrointestinal Clinic, Pediatric Hematology/Oncology Clinic, Pediatric Nephrology Clinic, Pediatric Orthopedic Clinic, Pediatric Rheumatology Clinic, Pediatric Scoliosis Clinic, Physical Therapy Clinic, Physician's Office, Podiatry Clinic, Prenatal Clinic, Pulmonary Clinic, Rheumatology Clinic, School or Prison Infirmary, Specimen Collection Area (Healthcare) Speech Therapy Clinic, Surgical Services Clinic, Well Baby Clinic, Wound Center, Wound Ostomy Continence Clinic, Endoscopy Suite, Radiology, includes Nuclear Medicine Mobile Blood Collection center, Mobile MRI/CTC, Community Locations, Blood Collection (Blood Drive Campaign) Home Care, Home-based Hospice, Location Outside Facility. Specimen Collection Area (Community) Non-Patient Care Locations, Administrative Areas CDC Locations and Descriptions, Assisted Living Area, Blood Bank, Central Sterile Supply Central Trash Area, Clinical Chemistry, Facility Grounds, General Laboratory, Hematology Laboratory, Histology/Surgical Pathology, Housekeeping/Environmental Services, Laundry Room, Microbiology Laboratory, Morgue/Autopsy Room, Pharmacy, Physical Plant Operations Center, Public Area in Facility, Serology Laboratory, Soiled Utility Area, Virology Laboratory, Facility Types—Non Healthcare: School/educational, Correctional, Military, Food service (such as a restaurant), Food processing (such as a manufacturer of food products), Pharmaceutical production, Commercial building/organization (such as a manufacturer where workers must apply protective creams routinely), and other facilities where spread of infections by hands is a concern.

### Long Term Care

More than 1.5 million residents in the United States currently reside in LTCF with an aging baby-boom generation more than 5.3 million LTCF residents are expected by 2030. In recent years, the acuity of illness of LTCF residents has increased, due to the average long-term care (LTC) resident having both advanced in age and having more complex health conditions. Seniors, particularly individuals with vulnerable health conditions that reside in LTCF are particularly susceptible to indoor air quality, and the associated environmental risks, as they may spend more than 95% of their time indoors, thereby increasing their exposure. These seniors are susceptible to airborne environmental risks through; (i) acquiring infections from airborne pathogens, (ii) response to airborne chemical stressors such as VOCs, and (iii) response to airborne particulate matter. Each of these environmental risks has unique response relationships; and the air purification systems and methods of the disclosure will lessen the airborne environmental risks (pathogens, particulates, chemicals) in one LTCF. The team will deploy an advanced air purification system (APS) that has been clinically effective at protecting the human embryo during IVF which represents the most sensitive physiological endpoint as it has no means or mechanisms of defense against environmental toxins. The air purification systems and methods of the disclosure reduce environmental health risks through improved indoor air quality and are an effective intervention as a disease prevention strategy in senior LTCF. Innovative state-of-art air quality control devices can deliver high quality air (total VOCs less than 20 ppb, less than 1 cfu/m³ biologicals, and ISO Class 6 particulates) that prevents disease and improves quality of life in senior LTCF.

Airborne pathogens contribute to the risk of LTCF residents developing HAIs, which now approach the levels seen in acute care hospital patients, impacting resident wellness, quality of life, and overall costs. In U.S. nursing homes an estimated 1.6-3.8 million HAIs occur per year, which result in an estimated $38-$137 million for antimicrobial therapy and $673 million-$2 billion for hospitalizations. In addition, as many as 380,000 people die from infections in LTCF every year. Historically, it has been understood that the source of the pathogens responsible for HAIs and general illnesses (influenza, pneumonia, tuberculosis, etc.) are associated with the patient and surface areas. Infection control protocols, patient preparation and hand washing protocols have been implemented and are helpful in reducing overall infection rates. Studies have demonstrated that even properly gowned healthcare workers and patients shed approximately 3,000-50,000 microorganisms per minute or 0.12 colony forming units (cfu) per cubic meter per minute. Approximately 10% of the shed microorganisms can be infectious. Recent literature indicates that significant sources of pathogens responsible for HAIs are airborne. Specifically, the data shows that pathogens responsible for HAIs, 69-80% are airborne. Infectious airborne pathogens include but are not limited to Clostridium difficile, antibiotic resistant MRSA, aspergillus, aspergillus, pseudomonas, streptococcus, staphylococcus, tuberculosis, smallpox and influenza. Many of the infectious pathogens can remain airborne and viable for weeks to months at a time, which could be eliminated with air purification.

VOC concentrations indoors are often found in levels 3-5 times greater than outdoor background air. Sources of VOCs include cleaners, disinfectants, aerosols, solvents, new plastics, dry cleaned materials, and building materials. Exposure to VOCs can induce a variety of health effects, including nose and throat discomfort, headache, dyspnea, nausea, fatigue, epistaxis, and dizziness, among other effects. It is well documented that the most susceptible populations to indoor VOCs include the elderly, individuals with compromised cardiac systems, individuals with pulmonary disorders, and other health conditions. VOC exposure and individual response is encompassed in clinical entities such as sick building syndrome in which exposure to airborne chemicals gives rise to disease.

Particulate matter (PM) is linked to: irritation of the airways, coughing, decreased lung function, aggravated asthma, irregular heartbeat, nonfatal heart attacks, and premature death for people with heart or lung disease. PM is generally classified by size as course (PM10) less than 10 micrometers, and fine (PM2.5) less than 2.5 micrometers. Some of the most compelling epidemiologic studies of ambient particulate matter have associated both acute and chronic mortality with fine particulate air pollution. Other studies have investigated indoor airborne particulates which suggest the concentrations are higher than outdoor particulates; and these indoor particulates also produced more inflammatory and allergic reactions, and accelerated blood coagulation.

Conventional physical filtration such as HEPA filtration is commonly used to reduce levels of airborne pathogens in healthcare settings, including LTCF. Physical filtration captures only those infectious pathogens of a certain size. Many bacterial and viral pathogens responsible for infections and illness are not captured. Those pathogens captured often continue to proliferate on the filter and thus remain a permanent threat to the patient, resident and staff in the space being protected by HEPA filtration. In addition, HEPA filtration does not remove airborne chemical agents, such as VOCs. Additionally, HEPA filtration does not guarantee the complete removal of particulates.

The air purification systems and methods of the disclosure do not capture pathogens through filtration, it removes all airborne bacterial and viral pathogens by destroying the DNA and RNA associated with the viable particulates. The technology renders the pathogens noninfectious on a single pass through the system while not disturbing patient, resident or personnel operations. Attempts by other researchers to control airborne infection rates have used multiple layers of HEPA filtration and Ultraviolet Germicidal Irradiation (UVGI), documenting a marked reduction in infection cases following the introduction of UVGI. The air purification systems and methods of the disclosure employ an advanced reformulated and optimized UVGI technology that was mathematically and genomically modeled to kill and inactivate infectious biologicals on a single pass through the system. A ten-year study of UVGI effectiveness on biological inactivation has demonstrated a statistical impact on clinical outcome data. The air purification system and methods of the disclosure also employ proprietary and patented technologies that include multiple levels of filtration, purification and inactivation media such that chemical (VOCs) and particulates are removed from the air, as previous research by Dr. Worrilow and others have found embryo sensitivity to VOCs.

Improved environmental air quality is clinically demonstrated with the air purification systems and methods of the disclosure. As evidence of environmental performance, traditional HEPA-filtered clinical environment offers mean viable particulates of 11,642 (0.3 micron) particulates/ft³, biologicals of 1,778 cfu/m³, and chemical TVOCs of 1,372 ppb. Whereas, with the air purification systems and methods of the disclosure the purified clinical environment is 5,410 (0.3 micron) particulates/ft³, <1 cfu/m³, and <5 ppb TVOC. Additionally, the clinical results in IVF outcomes due to air purification systems and methods of the disclosure demonstrate a 20.7% improvement (p=0.0001) in blastocyst conversion rate across 5,319 cycles, and statistical improvement through ongoing pregnancy rate, regardless of maternal age.

It is well established that airborne pathogens, particulates, and compounds such as VOCs are detrimental to human health. It is also established that the most susceptible populations to air pollution include the elderly, young children, individuals with compromised cardiac systems, individuals with pulmonary disorders, and other vulnerable health conditions. Seniors living in LTCF, with existing health conditions are particularly susceptible to indoor air quality. In the case of VOCs, concentrations indoors are often found in levels 3-5 times greater than outdoor background air. Exposure to VOCs can induce a variety of health effects, including nose and throat discomfort, headache, dyspnea, nausea, fatigue, epistaxis, and dizziness, among other effects. Sources of VOCs include cleaners, disinfectants, aerosols, solvents, new plastics, dry cleaned materials, and building materials. VOC concentrations are a key contributor to patient well-being for particularly vulnerable patients in LTCF. Additionally, PM has been linked to: irritation of the airways, coughing, decreased lung function, aggravated asthma, irregular heartbeat, nonfatal heart attacks, and premature death for people with heart or lung disease. Elevated levels of PM in LTCF may trigger health events in susceptible individuals. Airborne pathogens are well-studied as proliferating numerous communicable diseases. Reduced levels of airborne pathogens offer a prevention strategy for individuals living in long-term care facilities.

According to the Environmental Protection Agency (EPA), Americans spend 87% of their time indoors. The research team anticipates for seniors living in LTCF, these numbers likely exceed 95% of their time. While it is well studied that indoor air quality is a significant factor in the well-being of individuals, little has been done to improve indoor air quality in LTC settings. The air delivered through a conventional heating, ventilation, and air conditioning (HVAC) system to rooms with the air purification systems of the disclosure unit possesses UV and molecular media technologies with the capability to neutralize and remove 99.99% of biological and chemical contaminants. In addition, the air purification systems and methods of the disclosure removes PM to an ISO class 6 level.

### Life Sciences and Manufacturing

The air purification system and methods of the disclosure will reduce the levels of airborne biological, chemical, and particulate contamination and thus improve outcomes in all areas of basic, applied, commercial, industrial, biological, chemical research and chemical manufacturing. A technological paradigm shift and 24/7 live kill/remediation of ALL airborne bacterial, viral and chemical pathogens. The air purification system and methods of the disclosure eliminate bacterial, viral and chemical pathogens highly toxic to living cell culture, cell processing and production. The air purification system and methods of the disclosure will remove the variable of airborne pathogens for the optimal life sciences/living cell environment. It will remove 99.99% of biologicals and chemical contaminants with the system and methods of the disclosures. The air purification system and methods of the disclosure will comprehensively kill and remove all airborne pathogens: chemical, biological and particulate on a single pass with reduced air changes per hour (ACH) and energy use. All source and recirculated air is purified by the technology while producing no byproducts or ozone. The air purification system and methods of the disclosure operates 24/7 with full staff occupancy, active living cell culture, transfection and gene therapy processing and cell/vaccine manufacturing with no loss of cell culture, or life sciences processing space.

The air purification system and methods of the disclosure have successfully supported the in vitro culture and growth of hundreds of thousands of human embryos and has significantly impacted clinical outcomes.

The air purification system and methods of the disclosure are able to provide sterile air to the life sciences and clinical environment by killing/removing airborne biological, particulate and chemical pathogens from all source/outside and recirculated air from the space, thereby removing the variable of air from all ongoing processes. The air purification system and methods of the disclosure improve living cell culture, gene therapy and viral production processes. The air purification system and methods of the disclosure removes 99.99% of ALL embryotoxic airborne pathogens.

The air purification system and methods of the disclosure removes VOCs to virtually undetectable levels. The inventor has found that airborne contaminants suppress outcomes even in clean room environments, and even under the best protocols and practices HEPA/(ultra low particulate air) ULPA filtration, increased ACH and controlled air flow alone may not be enough. The system and methods of the disclosure provide a consistent and optimized, in vitro living cell/tissue culture and processing environment thereby improving cell viability, yield and efficacy.

In particular, low levels of VOCs and viable particulates, play a critical role in cellular and molecular toxicology, and in epigenetic processes. Low levels of chemical and biological airborne pathogens are impactful to successful cell culture and human embryogenesis. Good Manufacturing Practice (GMP) metrics do not address levels of toxic VOCs and viable particulates present in the processing space.

Varying partition coefficients of VOCs allow the chemical pathogens to readily enter processing or growth media and tissue culture oil. Varying biochemical structures and polarity create a challenge for remediation and allow a persistent presence in the culture or processing environment. Comprehensive removal of all VOCs in the in vitro culture of the human embryo was concomitant with statistically significant clinical outcomes. PPM levels of alcohol, toluene, styrene, aldehydes, formaldehyde and other toxic VOCs are common in the life sciences environment. PPB levels of VOCs cause toxicity by perturbing the path of DNA replication in living cells.

In addition, common fungal VOCs are toxic to the living cell process: Acetone Ethanol Isopropanol Toluene Styrene Methylene chloride Hexane 2—Heptanone Hexanol 2 Pentanol Methyl acetate Benzene 2—Propanyl acetate 2—Pentanone 2, 2—dimethylpropanol Acetic acid Ethyl acetate 1, and 4—Pentadiene Octanol.

The air purification system and methods of the disclosure provide source and recirculated air such that viable particulates and VOCs are delivered to the space at below detection levels, with comprehensive removal of the variable of airborne pathogens, airborne contamination and its impact on the living cell culture. This leads to protection of the cell culture process, increased process metrics, protection of staff, as well as risk mitigation and lowers the risk of contamination to the cellular product, and patients. The air purification system and methods of the disclosure provide cost avoidance by lessening compromised viability of cells, process, transfusion and associated steps of correction with the comprehensive kill of exhaust biological and viral load.

The system and methods of the disclosure relates to all areas of basic, applied, commercial, industrial, biological, and chemical research and manufacturing including, but not limited to, the following: biochemistry, bioinformatics, biotechnology, cell biology, chemical biology, cell therapy, cell and organ transplantation, developmental biology, ecology, endocrinology, epidemiology, evolution, genetics, gene therapy, genomics, gerontology, immunology, infectious diseases, microbiology, molecular biology, nephrology, neurology, ophthalmology, pediatrics, pharmacology, physiology, plant biology, psychiatry, public health, structural biology, surgery, urology, drug discovery, molecular therapeutics, epidemiology, carcinogenesis, inflammation, pain, nutrition, reproduction, virology, toxicology, pathology, dermatology, gastroenterology, musculoskeletal studies, pregnancy, pulmonary studies, breast cancer, cardiovascular studies, cerebrospinal research, allergy and asthma studies, hepatology, atherosclerosis, diabetes studies, hematology, oncology, osteoporosis studies, rheumatology studies, vaccine studies, circadian rhythms studies, proteome studies, respiratory research, thrombosis studies, anti-viral and anti-microbial and anti-parasite studies gene regulation studies, cell culture studies of all kinds, organ culture and transplant studies of all kinds, protein production studies, and in vitro and in vivo cell and organ growth and differentiation studies of all kinds.

The system and methods of the disclosure relates to all areas of basic, applied, commercial, industrial, biological, and chemical research and manufacturing including, but not limited to, all areas of marine and terrestrial basic, applied, commercial and industrial agricultural research and development. This includes most of the above mentioned areas as well as the following; developing improved viruses, microbes, cells, plants and animals, by natural and genetic engineering means for food production and other purposes; terrestrial and marine virus, microbe, plant, and animal diseases of all kinds; disease mechanisms and host-pathogen interactions; discovery, development, validation, production and use of antiviral agents, anti-microbial agents, antifungal agents, pesticides, vaccines of all kinds, plant and animal growth agents, and agricultural pharmaceutical agents of all kinds; agricultural ecology and toxicology; and products and services which are associated with the above described areas.

The system and methods of the disclosure relates to all areas of basic, applied, commercial, industrial, biological, and chemical research and manufacturing including, to a large number of medical areas, both human and veterinary and these include, but are not limited to, essentially all areas of basic, commercial, industrial, and applied human and veterinary medical research and development. These include, but are not limited to, the following; the pathogenesis and/or prevention and/or diagnosis and/or treatment, and/or cure of: infections and non-infectious diseases of almost all kinds; genetic and non-genetic disease of almost all kinds; nutritional diseases of almost all kinds; central nervous system diseases of almost all kinds, including psychiatric conditions; cancers and tumors of almost all kinds; cardiac diseases of almost all kinds; other tissue, organ, or cell diseases of almost all kinds; inflammation related diseases of almost all kinds; immunologic diseases of almost all kinds; toxic compound related diseases of almost all kinds; addictive diseases of almost all kinds; fetal and developmental diseases of almost all kinds; diagnostic tests for all such diseases; and products and services which are associated with the research and development and commercialization related to the diagnosis, prevention, control, treatment, or cure of such diseases, including vaccine development and commercialization.

The system and methods of the disclosure relates to all areas of basic, applied, commercial, industrial, biological, and chemical research and manufacturing including essentially all areas of human and veterinary medicine including those earlier described basic, commercial, or applied biological research areas. Further said present disclosure is related to essentially all areas of human and veterinary pharmaceutical and basic and applied and commercial and industrial and service research and development, drug discovery and validation and manufacturing, and the re-evaluation of existing drugs or drug rescue, including but not limited to the following areas; allergy and asthma, addiction, anesthesiology, anti-viral and anti-microbial and anti-fungal and anti-parasite agents, atherosclerosis, biochemistry, blood disorders, cancer and carcinogenesis, cardiology and cardiovascular, cerebrospinal, cell culture, cell therapy, dermatology, diabetes, development, dental, diagnostic, ecology, emergency medicine, endocrinology, epidemiology, gastroenterology, genetics, gene therapy, gerontology, hematology, hepatology, hypertension, immunology and autoimmune disorders, microbiology, molecular medicine, musculoskeletal disorders, nephrology, neurology and neuroscience, nuclear medicine, nutrition, oncology, ophthalmology, otolaryngology, pain, parasitology, pediatrics, pharmaceuticals, psychiatry, psychoses, public health, pulmonary medicine, reproduction, rheumatology, sports medicine, surgery, urology, vaccinology, and virology. Note that the above list of disclosure related human and veterinary medicine related areas, is by no means a complete list and is included to illustrate that the disclosure has very broad application in human medicine, veterinary medicine and other areas.

The system and methods of the disclosure relates to all areas of basic, applied, commercial, industrial, biological, and chemical research and manufacturing including essentially all of the steps in the process of human and veterinary drug discovery, characterization, optimization, validation, prescription and use. Here the term drug includes antiviral, and microbial, and antifungal compounds, as well as vaccines and other drug and bioactive molecule types of all kinds. Such disclosure related steps include, but are not limited to, the following: (a) the identification and characterization and development of one or more biological and/or non-biological drug target discovery systems. (b) Establishing quality control (QC) and quality assurance (QA) methods for each drug target discovery system. (c) The identification and characterization and development of one or more drug target candidates. (d) The identification and characterization and development of one or more biological and/or non-biological systems for screening drug candidates for the target. (e) Establishing QC and QA methods for the drug screening systems. (f) The identification and characterization of one or more drug candidates for each drug target and the evaluation of the specificity, and potency or efficacy and toxicity, of the candidate drug in the drug screening system. (g) The optimization of the drug candidate specificity, potency, and toxicity of the drug candidate in the screening system. (h) The identification and development of diagnostic tests for evaluating the drug candidate and target characteristics. (i) Establishing QC and QA methods for the scale up of synthesis of the drug candidate. (j) The identification, characterization, and development of one or more biological and non-biological systems for further evaluating and optimizing the drug candidate specificity, potency, efficacy, and toxicity. (k) Establishing QC and QA methods for the biological and non-biological systems. (l) The identification, characterization, development, and manufacturing of one or more biological systems to evaluate pharmacodynamic and pharmacokinetic characteristics of the candidate drug. (m) Establishing QC and QA methods for this biological system. (n) The use of the biological system to optimize the pharmacodynamic and pharmacokinetic characteristics of the candidate drug. (o) The development of human organism related diagnostic assays for evaluation of the drug candidate pharmacodynamic and pharmacokinetic characteristics, including the specificity, potency, and toxicity, in human organisms. (p) The use of the human related diagnostics to screen human populations for inclusion in clinical trials, and to define the target human population that the drug is effective and safe for. (q) The use of the human organism related diagnostic methods to monitor the pharmacodynamic and pharmacokinetic characteristics of the drug candidate in human trial participants, and for disease monitoring during the clinical trial and after. (t) The use of human related and other diagnostic methods for QC and QA of the drug manufacturing scale up and manufacturing process. (u) The use of human related diagnostic methods to prescribe the drug and monitor the drug effectiveness during treatment and for disease monitoring before, during, and after drug treatment. (v) The use of the human related diagnostic methods to monitor the long term pharmacodynamic and toxicity characteristics.

The system and methods of the disclosure relates to all areas of basic, applied commercial, industrial, biological, and chemical research and manufacturing including and applied and service use of the following: prokaryote cells and cell cultures, and eukaryotic cells and cultured primary and continuous cells. Eukaryotic organisms, organs, and tissues, as well as organs and tissues cultured in vitro. Such disclosure related uses include, but are not limited to, the following: The use of prokaryotic cells grown in large quantities to produce a wide variety of products, including drugs; the use of primary and continuous eukaryotic cell cultures grown in large quantities, to produce a wide variety of products, including drugs; and the use of prokaryotic and eukaryotic cultured cells for a wide variety of basic, commercial, applied, industrial, research, development and service applications. Such eukaryotic cells include primary and continuous stem cell lines and the use of genetically modified microbial, plant, fungal or animal organisms or cells for various aspects of drug, biochemical, and bioproduct or food production. The use of organisms and genetically modified organisms includes interfering RNA treatment of such organisms.

In certain embodiments, the cell type is for example, a eukaryotic cell type, a prokaryotic cell type, a plant cell type, a bacterial cell type, a pathogenic bacterial cell type, a yeast cell type, a fungal cell type, a mammalian cell type, a human cell type, an in vitro grown cell type, an immortalized cell line type, an in vivo grown cell type, an infectious organism or agent infected cell type, a virus infected cell type, a genetically modified cell type, and/or an in vivo or in vitro cell type used for producing or manufacturing a pharmaceutical agent or protein or small molecule or lipid.

The disclosure also relates broadly to basic, commercial, applied, and industrial research and development aspects of toxicology. Many if not most of the above described disclosure related areas also relate directly or indirectly to toxicology, as well as to the areas of quality control and monitoring of water, food quality, nutrition, public health, marine and terrestrial ecology, forensics, and many kinds of technology development, QC, QA, and various services associated with one or more of the above.

Note that the above description of the disclosure related areas is not a complete list, and represents only a small fraction of the actual disclosure related areas.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989; “Methods in Enzymology: Guide to Molecular Cloning Techniques”, Academic Press, Berger, S. L. and A. R. Kimmel eds., 1987; Scopes, Protein Purification: Principles and Practice, Springer-Verlog (1994); Ausubel et al., Current Protocols in Molecular Biology. Greene Publishing Associates and Wiley-Interscience John Wiley and Sons, New York, 1987.

The system and methods of the disclosure relates to all areas of basic, applied, commercial, industrial, biological, and chemical research and manufacturing including applied and service use of the following: “antibody” is used in the broadest sense and specifically covers, for example, monoclonal antibodies (including agonist antibodies), antibody compositions with polyepitopic specificity, single chain antibodies, and fragments of antibodies. The term “monoclonal antibody” as used herein refers to an antibody obtained from a population of substantially homogeneous antibodies, i.e., the individual antibodies comprising the population are identical except for possible naturally-occurring mutations that may be present in minor amounts.

The system and methods of the disclosure further relates broadly to basic, commercial, applied, industrial, research and development, manufacturing, applied and service use of the following: “Biological activity” in the context of an antibody or another agonist that can be identified by the screening assays disclosed herein (e.g., an organic or inorganic small molecule, peptide, etc.) is used to refer to the ability of such molecules to invoke one or more of the effects listed herein in connection with the definition of a “therapeutically effective amount.” In a specific embodiment, “biological activity” is the ability to inhibit neoplastic cell growth or proliferation. A preferred biological activity is inhibition, including slowing or complete stopping, of the growth of a target tumor (e.g., cancer) cell. Another preferred biological activity is cytotoxic activity resulting in the death of the target tumor (e.g., cancer) cell. Yet another preferred biological activity is the induction of apoptosis of a target tumor (e.g., cancer) cell.

The system and methods of the disclosure relates to all areas of basic, applied, commercial, industrial, biological, and chemical research and manufacturing including applied and service use of the following: “immunological activity” means immunological cross-reactivity with at least one epitope of a polypeptide “Immunological cross-reactivity” as used herein means that the candidate polypeptide is capable of competitively inhibiting the qualitative biological activity of a polypeptide having this activity with polyclonal antisera raised against the known active polypeptide. Such antisera are prepared in conventional fashion by injecting goats or rabbits, for example, subcutaneously with the known active analogue in complete Freund's adjuvant, followed by booster intraperitoneal or subcutaneous injection in incomplete Freunds. The immunological cross-reactivity preferably is “specific”, which means that the binding affinity of the immunologically cross-reactive molecule (e.g., antibody) identified, to the corresponding PRO polypeptide is significantly higher (preferably at least about 2-times, more preferably at least about 4-times, even more preferably at least about 6-times, most preferably at least about 8-times higher) than the binding affinity of that molecule to any other known native polypeptide.

The system and methods of the disclosure relates to all areas of basic, applied, commercial, industrial, biological, and chemical research and manufacturing including applied and service use of the following: “cytotoxic agent” as used herein refers to a substance that inhibits or prevents the function of cells and/or causes destruction of cells. The term is intended to include radioactive isotopes (e.g., I131, I125, Y90 and Re186), chemotherapeutic agents, and toxins such as enzymatically active toxins of bacterial, fungal, plant or animal origin, or fragments thereof.

The system and methods of the disclosure relates to all areas of basic, applied, commercial, industrial, biological, and chemical research and manufacturing including applied and service use of the following: A “chemotherapeutic agent” is a chemical compound useful in the treatment of tumor, e.g., cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), doxetaxel (Taxotere, Rhone-Poulenc Rorer, Antony, Rnace), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins, melphalan and other related nitrogen mustards. Also included in this definition are hormonal agents that act to regulate or inhibit hormone action on tumors such as tamoxifen and onapristone.

The system and methods of the disclosure relates to all areas of basic, applied, commercial, industrial, biological, and chemical research and manufacturing including applied and service use of the following: “growth inhibitory agent” when used herein refers to a compound or composition which inhibits growth of a cell, especially tumor, e.g., cancer cell, either in vitro or in vivo. Thus, the growth inhibitory agent is one which significantly reduces the percentage of the target cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al., (WB Saunders: Philadelphia, 1995), especially p. 13.

The system and methods of the disclosure relates to all areas of basic, applied, commercial, industrial, biological, and chemical research and manufacturing including applied and service use of the following: term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine: insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β: insulin-like growth factor-I and -II: erythropoietin (EPO); osteoinductive factors: interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-Iα, IL-2, IL-3, IL-4, IL-5, IL-6, IL-7, IL-8, IL-9, IL-11, IL-12, a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The system and methods of the disclosure relates to all areas of basic, applied, commercial, industrial, biological, and chemical research and manufacturing including applied and service use of the following: “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy”, Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this disclosure include, but are not limited to, phosphate-containing prodrugs, thiophosphate-containing prodrugs, glycosylated prodrugs or optionally substituted phenylacetamide-containing prodrugs, 5-fluorocytosine and other 5-fluorouridine prodrugs which can be derivatized into a prodrug form for use in this disclosure include, but are not limited to, those chemotherapeutic agents described above.

The system and methods of the disclosure relates to all areas of basic, applied, commercial, industrial, biological, and chemical research and manufacturing including applied and service use of the following: “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a polypeptide may comprise contacting a tumor cell with a candidate agonist molecule and measuring the inhibition of tumor cell growth.

The system and methods of the disclosure relates to all areas of basic, applied, commercial, industrial, biological, and chemical research and manufacturing including applied and service use of the following: “label” when used herein refers to a detectable compound or composition which is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition which is detectable. The label may also be a non-detectable entity such as a toxin.

### Air Purifier

The air purification technology and methods of the disclosure will reduce the levels of airborne biological, chemical, and particulate contamination and thus improve outcomes in all areas of basic, applied, commercial, industrial, biological, and chemical research and manufacturing, as well as hospital settings, and long-term care facilities, as set forth herein. Accordingly, air characterized by very high purity and methods of making and using such air, are provided.

In one aspect of the present disclosure, air is provided, characterized by a TVOC content of from less than 5 ppb to about 500 ppb, a Biologicals content of from less than 1 CFU/M³ to 150 CFU/M³ and a Particulate content of from about 1,000 0.3 μm particles per ft³ to about 50,000 0.3 μm particles per ft³, or from about 600 0.5 μm particles per ft³ to about 500,000 0.5 μm particles per ft³.

Another aspect of the present disclosure is a method of achieving an IVF clinical pregnancy rate of at least 50%. The method includes performing multiple WF cycles in an WF laboratory having air characterized by a TVOC content of from less than 5 ppb to about 500 ppb, a Biologicals content of from less than 1 CFU/M³ to 150 CFU/M³ and a Particulate content of from about 1,000 0.3 μm particles per ft³ to about 50,000 0.3 μm particles per ft³, or from about 600 0.5 μm particles per ft³ to about 500,000 0.5 μm particles per ft³.

Another aspect of the present disclosure is a method of purifying air, including providing an air flow path through a housing for the flow of air in a downstream direction, filtering the air through oxidizing and adsorbing VOC pre-filtration within the housing, filtering the air through UV filtration within the housing, downstream from the oxidizing and adsorbing VOC pre-filtration and filtering the air through final particulate filtration within the housing, downstream from the UV filtration.

The disclosure provides a method comprising: providing purified air, wherein the purified air is characterized by: a. a Biologicals content of from less than about 1 CFU/M³ to 150 CFU/M³; and b. a Particulate content of from about 1,000 0.3 μm particles per ft³ of air to about 30,000 0.3 μm particles per ft³ of air, or from about 600 0.5 μm particles per ft³ of air to about 10,000 0.5 μm particles per ft³ of air, and performing at least one WF procedure in said purified air.

The disclosure provides a method comprising: providing purified air, wherein the purified air is characterized by: a. a TVOC content of from less than about 5 ppb to about 500 ppb; and b. a Particulate content of from about 1,000 0.3 μm particles per ft3 of air to about 30,000 0.3 μm particles per ft3 of air, or from about 600 0.5 μm particles per ft3 of air to about 10,000 0.5 μm particles per ft3 of air, and performing at least one WF procedure in said purified air.

The disclosure provides a method comprising: providing purified air, wherein the purified air is characterized by: a. a TVOC content of from less than about 5 ppb to about 500 ppb; and b. a Biologicals content of from less than about 1 CFU/M³ to 150 CFU/M³, and performing at least one IVF procedure in said purified air. The disclosure provides a method wherein the at least one IVF procedure comprises a plurality of IVF procedures.

The disclosure provides a method of purifying air, the method comprising the steps of: providing an air purifier; providing source air which is to be purified to the air purifier; purifying the source air with the air purifier, thereby providing purified air, wherein the purified air has characteristics selected from the group consisting of: a. a TVOC content of less than about 5 ppb; b. a Biologicals content of less than about 1 CFU/M³; c. a Particulate content from about 1,000 0.3 μm particles per ft³ to about 10,500 0.3 μm particles per ft³, or from about 600 0.5 μm particles per ft³ to about 1,000 0.5 μm particles per ft³; and d. combinations thereof. The disclosure provides a method wherein the source air that is to be treated by the air purifier is pre-conditioned and stable. The disclosure provides a method wherein the source air that is to be treated by the air purifier air has a constant air pressure, constant air flow rate, volume, temperature, and/or humidity. The disclosure provides a method wherein the source air that is to be treated by the air purifier has a temperature of between about 50° F. and about 78° F. The disclosure provides a method wherein the source air that is to be treated by the air purifier has a humidity of between about 20% and about 80%. The disclosure provides a method wherein the the source air that is to be treated by the air purifier has a temperature of between about 50° F. and about 78° F. and a humidity of between about 20% and about 80%. The disclosure provides a method wherein the source air that is to be treated by the air purifier has a temperature of between about 50° F. and about 72° F. The disclosure provides a method wherein the source air that is to be treated by the air purifier has a relative humidity of between about 30% and about 70%. The disclosure provides a method wherein the source air that is to be treated by the air purifier has a temperature of between about 50° F. and about 72° F. and a relative humidity of between about 30% and about 70%. The disclosure provides a method wherein the air purifier comprises: a. a housing having an inlet for receiving air and an outlet for exhausting air, the housing providing an air flow path for the flow of air in a downstream direction, from the inlet towards the outlet; b. oxidizing and adsorbing VOC pre-filtration within the housing downstream from the inlet; c. UV filtration within the housing downstream from the VOC pre-filtration; and d. final particulate filtration within the housing downstream from the UV filtration. The disclosure provides a method wherein the purifying step comprises: a. providing an air flow path through a housing for the flow of air in a downstream direction; b. filtering the air through oxidizing and adsorbing VOC pre-filtration within the housing; c. filtering the air through UV filtration within the housing, downstream from the oxidizing and adsorbing VOC pre-filtration; and d. filtering the air through final particulate filtration within the housing, downstream from the UV filtration. The disclosure provides a method further comprising the step of filtering the air through particulate pre-filtration within the housing, upstream from the VOC pre-filtration. The disclosure provides a method wherein the VOC pre-filtration comprises bonded carbon. The disclosure provides a method further comprising the step of filtering the air through oxidizing and adsorbing VOC post-filtration within the housing, downstream from the UV filtration and upstream from the final particulate filtration. The disclosure provides a method wherein both the VOC pre-filtration and VOC post-filtration comprise bonded media. The disclosure provides a method wherein the VOC pre-filtration comprises one or more filters containing media selected from the group consisting of blended carbon, KMnO₄, and combinations thereof. The disclosure provides a method further comprising the steps of filtering the air through particulate pre-filtration within the housing, upstream from the VOC pre-filtration and filtering the air through oxidizing and adsorbing VOC post-filtration within the housing, downstream from the UV filtration and upstream from the final particulate filtration. The disclosure provides a method wherein the VOC pre-filtration and VOC post-filtration comprise one or more filters containing media selected from the group consisting of blended carbon, KMnO₄, and combinations thereof. The disclosure provides a method wherein the final particulate filtration includes one or more filters selected from the group consisting of HEPA filters and ULPA filters. The disclosure provides a method wherein the air purifier comprises: a. a housing having an inlet for receiving air and an outlet for exhausting air, the housing providing an air flow path for the flow of air in a downstream direction, from the inlet towards the outlet; b. UV filtration within the housing downstream from the inlet; c. oxidizing and adsorbing VOC post-filtration within the housing downstream from the UV filtration; and d. final particulate filtration within the housing downstream from the VOC post-filtration. The disclosure provides a method wherein the air purifier further comprises a booster fan within the housing, downstream from the inlet and upstream from the UV filtration. The disclosure provides a method wherein the final particulate filtration includes one or more filters selected from the group consisting of HEPA filters and ULPA filters. The disclosure provides a method wherein the particulate filtration includes one or more filters selected from the group consisting of HEPA filters and ULPA filters. The disclosure provides a method wherein the final particulate filtration includes one or more filters selected from the group consisting of HEPA filters and ULPA filters.

The disclosure provides an air purifier comprising: a. a housing having an inlet for receiving air and an outlet for exhausting air, the housing providing an air flow path for the flow of air in a downstream direction, from the inlet towards the outlet; b. a pre-filter; c. oxidizing and adsorbing VOC filter within the housing; d. particulate filtration; e. UV filtration within the housing; and f. optionally a final particulate filtration within the housing downstream from the UV filtration. The disclosure provides an air purifier wherein the air purifier is an in-room unit. The disclosure provides an air purifier wherein the air purifier is in line with the ductwork of an HVAC system or AHU. The disclosure provides an air purifier wherein the VOC filtration comprises bonded carbon. The disclosure provides an air purifier wherein the VOC filtration comprises bonded media. The disclosure provides an air purifier wherein the VOC filtration comprises one or more filters containing media selected from the group consisting of blended carbon, KMnO₄, and combinations thereof. The disclosure provides an air purifier wherein the UV filtration includes at least one UV source. The disclosure provides an air purifier wherein the UV filtration includes a plurality of UV sources. The disclosure provides an air purifier wherein the UV filtration includes at least one UVC source, capable of generating UV radiation at a wavelength of from 220 nm to 288 nm. The disclosure provides an air purifier wherein the UV filtration includes at least one UVC source capable of generating UV radiation at a wavelength of 254 or 260 nm. The disclosure provides an air purifier wherein the particulate filtration includes one or more filters selected from the group consisting of HEPA filters and ULPA filters. The disclosure provides an air purifier wherein the final particulate filtration includes one or more filters selected from the group consisting of HEPA filters and ULPA filters.

The disclosure provides an air purifier comprising: a. a housing having an inlet for receiving air and an outlet for exhausting air, the housing providing an air flow path for the flow of air in a downstream direction, from the inlet towards the outlet; b. a pre-filter; c. UV filtration within the housing; and d. optionally a final particulate filtration within the housing downstream from the UV filtration. The disclosure provides an air purifier wherein the air purifier is an in-room unit. The disclosure provides an air purifier wherein the air purifier is in line with the ductwork of a HVAC system or AHU. The disclosure provides an air purifier wherein the UV filtration includes at least one UV source. The disclosure provides an air purifier wherein the UV filtration includes a plurality of UV sources. The disclosure provides an air purifier wherein the UV filtration includes at least one UVC source, capable of generating UV radiation at a wavelength of from 220 nm to 288 nm. The disclosure provides an air purifier wherein the UV filtration includes at least one UVC sources are capable of generating UV radiation at a wavelength of 254 or 260 nm. The disclosure provides an air purifier wherein the particulate filtration includes one or more filters selected from the group consisting of HEPA filters and ULPA filters. The disclosure provides an air purifier wherein the final particulate filtration includes one or more filters selected from the group consisting of HEPA filters and ULPA filters.

The disclosure provides an air purifier comprising: a. a housing having an inlet for receiving air and an outlet for exhausting air, the housing providing an air flow path for the flow of air in a downstream direction, from the inlet towards the outlet; b. a pre-filter; c. UV filtration within the housing; d. oxidizing and adsorbing VOC filter within the housing; e. particulate filtration; f. optionally a final particulate filtration within the housing. The disclosure provides an air purifier wherein the air purifier is an in-room unit. The disclosure provides an air purifier wherein the air purifier is in line with the ductwork of a HVAC system or AHU. The disclosure provides an air purifier wherein the VOC filtration comprises bonded carbon. The disclosure provides an air purifier wherein the VOC filtration comprises bonded media. The disclosure provides an air purifier wherein the VOC filtration comprises one or more filters containing media selected from the group consisting of blended carbon, KMnO₄, and combinations thereof. The disclosure provides an air purifier wherein the UV filtration includes at least one UV source. The disclosure provides an air purifier wherein the UV filtration includes a plurality of UV sources. The disclosure provides an air purifier wherein the UV filtration includes at least one UVC source, capable of generating UV radiation at a wavelength of from 220 nm to 288 nm. The disclosure provides an air purifier wherein the UV filtration includes at least one UVC source capable of generating UV radiation at a wavelength of 254 or 260 nm. The disclosure provides an air purifier wherein the particulate filtration includes one or more filters selected from the group consisting of HEPA filters and ULPA filters. The disclosure provides an air purifier wherein the final particulate filtration includes one or more filters selected from the group consisting of HEPA filters and ULPA filters.

The disclosure provides an air purifier comprising: a. a housing having an inlet for receiving air and an outlet for exhausting air, the housing providing an air flow path for the flow of air in a downstream direction, from the inlet towards the outlet; b. a pre-filter; c. oxidizing and adsorbing VOC filter within the housing; d. particulate filtration; e. UV filtration within the housing; f. optionally a final particulate filtration within the housing. The disclosure provides an air purifier wherein the air purifier is an in-room unit. The air purifier of the disclosure wherein the air purifier is in line with the ductwork of a HVAC system or AHU. The disclosure provides an air purifier wherein the VOC filtration comprises bonded carbon. The air purifier of the disclosure wherein the VOC filtration comprises bonded media. The disclosure provides an air purifier wherein the VOC filtration comprises one or more filters containing media selected from the group consisting of blended carbon, KMnO₄, and combinations thereof. The disclosure provides an air purifier wherein the UV filtration includes at least one UV source. The disclosure provides an air purifier wherein the UV filtration includes a plurality of UV sources. The disclosure provides an air purifier wherein the UV filtration includes at least one UVC source, capable of generating UV radiation at a wavelength of from 220 nm to 288 nm. The disclosure provides an air purifier wherein the UV filtration includes at least one UVC source capable of generating UV radiation at a wavelength of 254 or 260 nm. The disclosure provides an air purifier wherein the particulate filtration includes one or more filters selected from the group consisting of HEPA filters and ULPA filters. The disclosure provides an air purifier wherein the final particulate filtration includes one or more filters selected from the group consisting of HEPA filters and ULPA filters.

The disclosure provides an air purifier comprising: a. a housing having an inlet for receiving air and an outlet for exhausting air, the housing providing an air flow path for the flow of air in a downstream direction, from the inlet towards the outlet; b. a pre-filter; c. particulate filtration; d. oxidizing and adsorbing VOC filter within the housing; e. UV filtration within the housing; f. optionally a final particulate filtration within the housing. The disclosure provides an air purifier wherein the air purifier is an in-room unit. The disclosure provides an air purifier wherein the air purifier is in line with the ductwork of a HVAC system or AHU. The disclosure provides an air purifier wherein the VOC filtration comprises bonded carbon. The air purifier of the disclosure wherein the VOC filtration comprises bonded media. The disclosure provides an air purifier wherein the VOC filtration comprises one or more filters containing media selected from the group consisting of blended carbon, KMnO₄, and combinations thereof. The disclosure provides an air purifier wherein the UV filtration includes at least one UV source. The disclosure provides an air purifier wherein the UV filtration includes a plurality of UV sources. The disclosure provides an air purifier wherein the UV filtration includes at least one UVC source, capable of generating UV radiation at a wavelength of from 220 nm to 288 nm. The disclosure provides an air purifier wherein the UV filtration includes at least one UVC source capable of generating UV radiation at a wavelength of 254 or 260 nm. The disclosure provides an air purifier wherein the particulate filtration includes one or more filters selected from the group consisting of HEPA filters and ULPA filters. The disclosure provides an air purifier wherein the final particulate filtration includes one or more filters selected from the group consisting of HEPA filters and ULPA filters.

The disclosure provides an air purifier comprising: a. a housing having an inlet for receiving air and an outlet for exhausting air, the housing providing an air flow path for the flow of air in a downstream direction, from the inlet towards the outlet; b. a pre-filter; c. particulate filtration; d. oxidizing and adsorbing VOC filter within the housing; e. UV filtration within the housing; f. optionally a final particulate filtration within the housing. The disclosure provides an air purifier wherein the air purifier is an in-room unit. The disclosure provides an air purifier wherein the air purifier is in line with the ductwork of a HVAC system or AHU. The disclosure provides an air purifier wherein the VOC filtration comprises bonded carbon. The disclosure provides an air purifier wherein the VOC filtration comprises bonded media. The disclosure provides an air purifier wherein the VOC filtration comprises one or more filters containing media selected from the group consisting of blended carbon, KMnO₄, and combinations thereof. The disclosure provides an air purifier wherein the UV filtration includes at least one UV source. The disclosure provides an air purifier wherein the UV filtration includes a plurality of UV sources. The disclosure provides an air purifier wherein the UV filtration includes at least one UVC source, capable of generating UV radiation at a wavelength of from 220 nm to 288 nm. The disclosure provides an air purifier wherein the UV filtration includes at least one UVC source capable of generating UV radiation at a wavelength of 254 or 260 nm. The disclosure provides an air purifier wherein the particulate filtration includes one or more filters selected from the group consisting of HEPA filters and ULPA filters. The disclosure provides an air purifier wherein the final particulate filtration includes one or more filters selected from the group consisting of HEPA filters and ULPA filters.

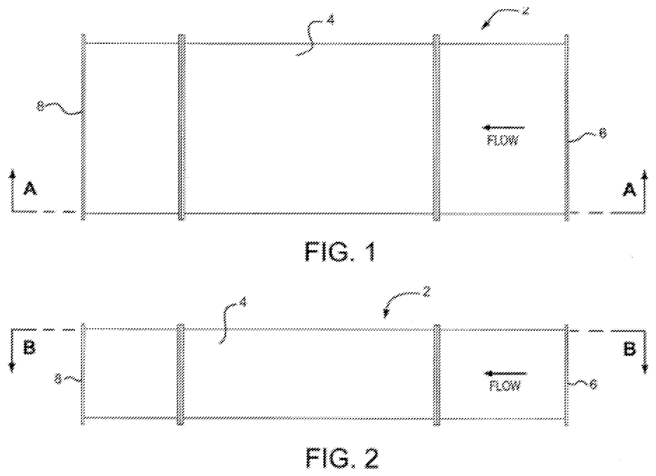

Referring now in detail to the various figures of the drawings wherein like reference numerals refer to like parts, there are shown in FIGS. 1 and 2 top and side views, respectively, of an air purifier 2 according to the present disclosure. As illustrated, the air purifier 2 includes a substantially rectangular cuboid housing 4 having an inlet 6 for receiving air and an outlet 8 for exhausting air. The term “air” as used herein broadly refers to a gas or gaseous mixture that may be safely breathed by mammals and/or that can serve as a source gas or gaseous mixture towards an IVF laboratory. The housing 4 provides an air flow path for the flow of air in a downstream direction, i.e., from the inlet 6 towards the outlet 8. The term “housing” as used herein refers to any conduit, chamber and/or enclosure, or a plurality of conduits, chambers and/or enclosures coupled to one another, providing an air flow path within. Thus, the “housing” could include, e.g., ductwork of an existing HVAC system or AHU. In certain embodiments, the air purifier is a stand-alone unit which purifies in—room air. In certain embodiments, the air purifier is an in-room unit that purifies in-room air.

Although the housing 4 is preferably substantially rectangular cuboid, as shown in FIGS. 1 and 2, it need not be limited to any particular shape. Moreover, it may include inner curves, bends and/or other contours, whereby the air flow path would follow such curves, bends and/or other contours. Preferably, however, the air flow path is substantially straight, as it is in the embodiment of the housing 4 shown in FIGS. 1 and 2.

The air purifier 2 is preferably adapted to be installed into an existing HVAC system or AHU. In an alternative embodiment, an air purifier according to the present disclosure may function as a stand-alone unit, i.e., one that is not part of an HVAC system or AHU. In certain embodiments, the air purifier is a stand-alone unit which purifies in—room air. In certain embodiments, the air purifier is an in-room unit that purifies in-room air. In certain embodiments, the air purifier is an in-room unit that is an in-ceiling unit. In certain embodiments, the air purifier is an in-room unit that is a ceiling unit. In certain embodiments, the air purifier is an in-room unit that is an in-ceiling unit that purifies in-room air. In certain embodiments, the air purifier is an in-room unit that is a ceiling unit that purifies in-room air. An exemplary housing 4 may be a substantially rectangular cuboid having dimensions of approximately 11 ft. long by 4 ft. wide by 2 ft. high. Such dimensions would diffuse or spread out the air through the air purifier 2 so as to provide sufficient resonance time for the air through each of the filtration media discussed infra. A skilled artisan understands, however, that the foregoing exemplary shape and size parameters are merely illustrative, and may be changed, even substantially, depending on the circumstances or application. For example, in some applications, the air purifier 2 may be about 6 ft. long.

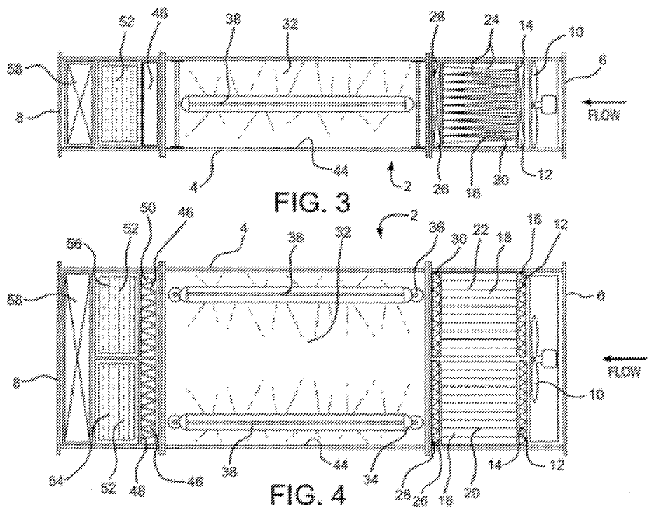

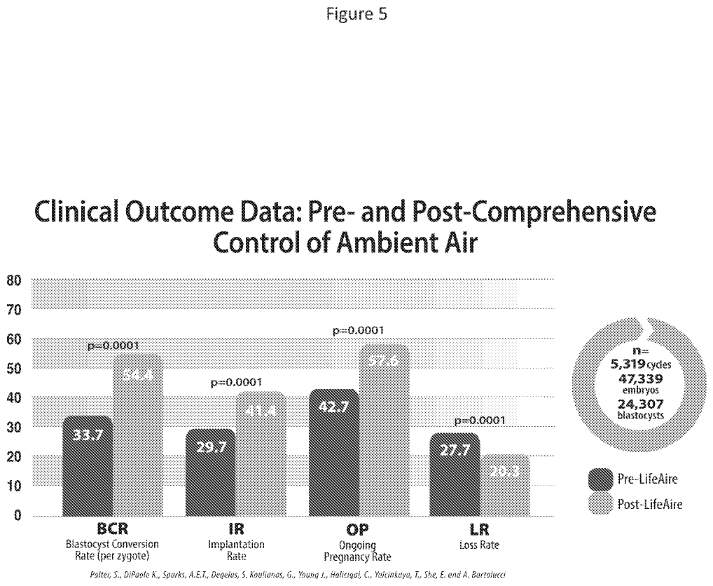

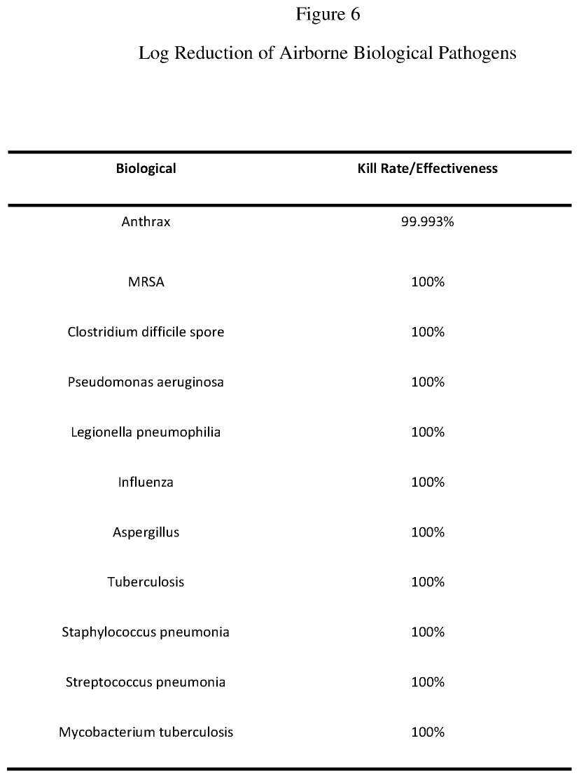

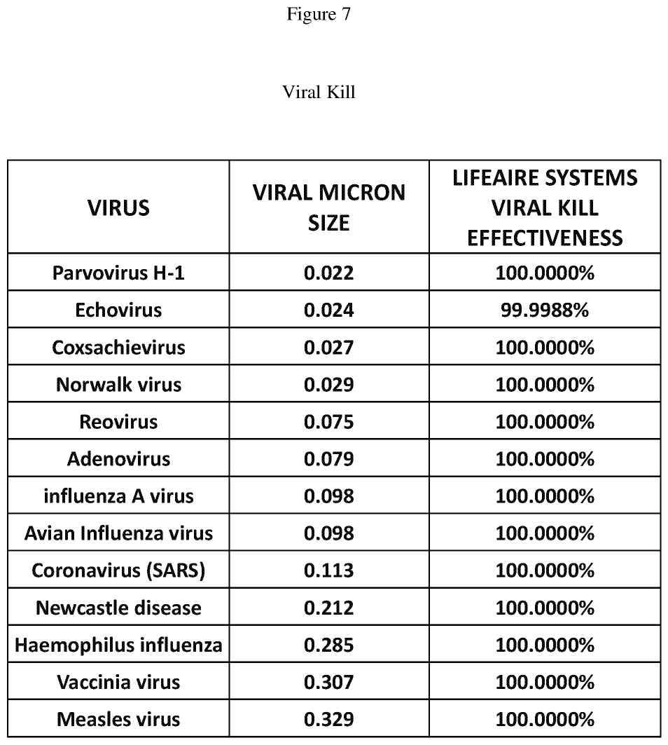

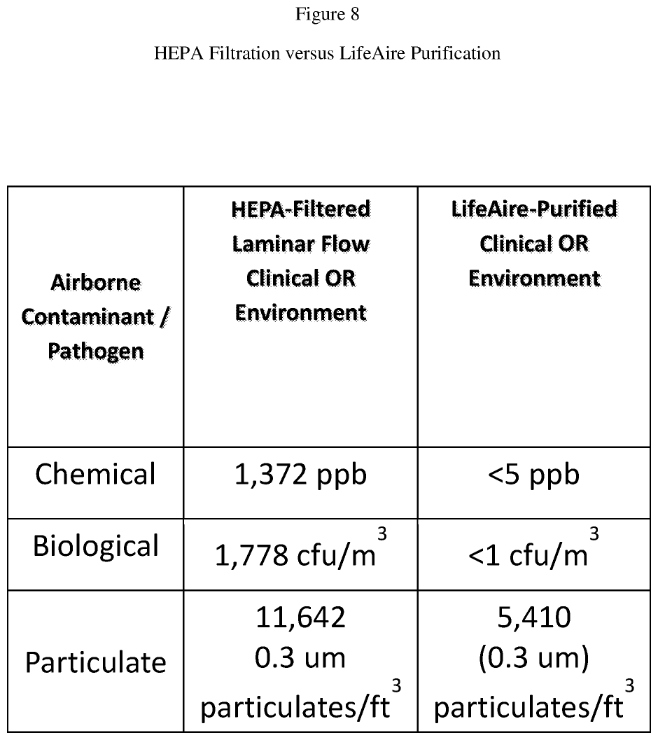

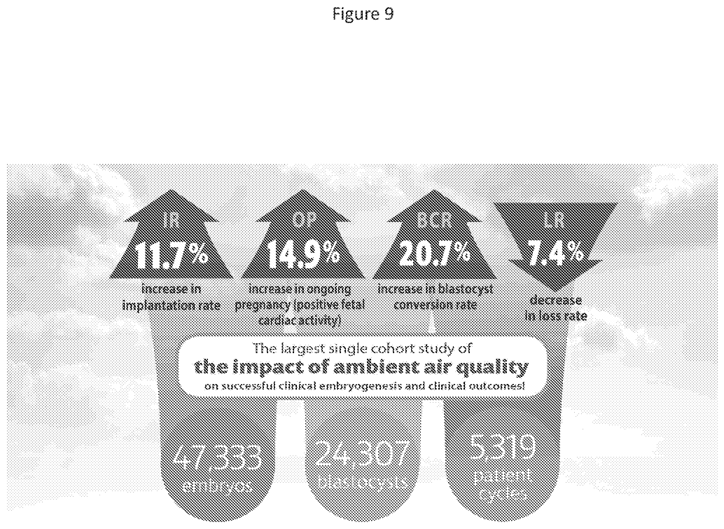

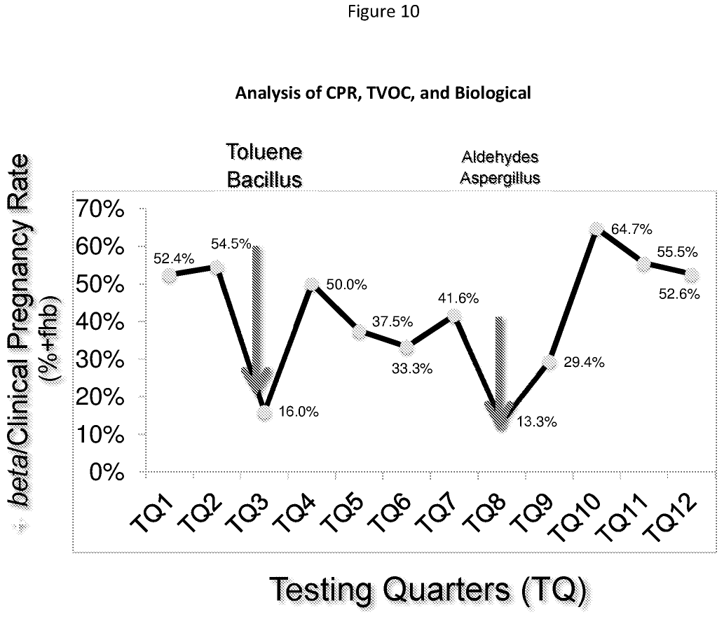

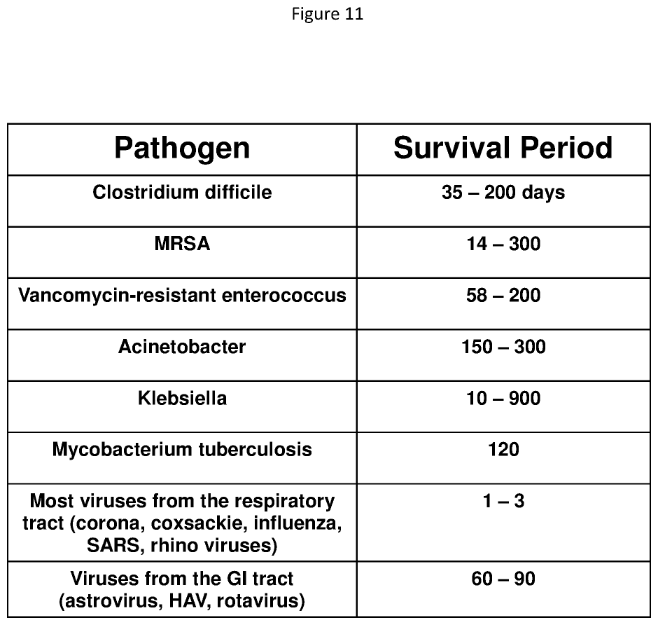

Referring now to FIG. 3, there is shown an internal view of the air purifier 2 along the plane defined by section line A-A of FIG. 1. In FIG. 4, there is shown an internal view of the air purifier 2 along the plane defined by section line B-B of FIG. 2.

To obtain optimal air quality, e.g., suitable for an IVF laboratory, the immediate upstream source air which is entering the air purifier 2 and which is to be treated by the air purifier 2 should be pre-conditioned and stable, i.e., moderate both in terms of, for example, temperature and humidity. In exemplary embodiments, the source air that is to be treated by the air purifier is pre-conditioned and stable. In exemplary embodiments, the source air that is to be treated by the air purifier has a constant air pressure, air flow rate, volume, temperature, and/or humidity. In exemplary embodiments, the source air that is to be treated by the air purifier has has a temperature of between about 50° F. and about 78° F. In certain embodiments, the source air that is to be treated by the air purifier has a humidity of between about 20% and about 80%.

In certain embodiments, the source air that is to be treated by the air purifier has a temperature of between about 50° F. to about 72° F. and/or a humidity of between about 30% to about 70% relative humidity. In exemplary embodiments, the source air that is to be treated by the air purifier has a temperature of between about 68° F. and about 75° F. and a humidity of between about 45% and about 55% relative humidity. In an exemplary embodiment, the source air is recirculated in-room air.

In exemplary embodiments, the source air that is treated by the air purifier 2 should have a temperature of between about 68° F. and about 75° F., and/or a humidity of between about 45% and about 55%. Additionally, the air flow rate through the air purifier 2 should, for example, preferably be between about 250 cubic ft/min (“CFM”) and below 2000 CFM. In exemplary embodiments, the air flow rate is about 200 CFM, about 250 CFM, about 300 CFM, about 350 CFM, about 400 CFM, about 450 CFM, about 500 CFM, about 550 CFM, about 600 CFM, about 650 CFM, about 700 CFM, about 750 CFM, about 800 CFM, about 850 CFM, about 900 CFM, about 950 CFM, about 1000 CFM, about 1500 CFM, about 2000 CFM, about 2500 CFM, about 3000 CFM, about 3500 CFM, about 4000 CFM, about 4500 CFM, about 5000 CFM, or about 5500 CFM. This preferred flow rate is intended to provide sufficient resonance time for the air through each of the filtration media discussed infra. The term “filtration” as used herein, broadly covers one or more devices that treat air, such as by trapping, removing, deactivating and/or destroying contaminants therefrom.

The source air may be provided by, for example, a HVAC system, an AHU, or may be in-room air, which may be optionally recirculated.

In order to provide an adequate air flow rate through the air purifier 2, it may be helpful (although not always necessary) to include a booster fan 10 downstream from the inlet 6. The booster fan 10 may be coupled to a control system (not shown) that measures the air flow rate and triggers the booster fan 10 as needed, to maintain the desired air flow rate. In an alternative embodiment (not shown), a booster fan may not be included, and adequate air flow rate may be provided and maintained by other means, e.g., a blower in an HVAC system or AHU into which the air purifier 2 is installed.

Downstream from the inlet 6 is particulate pre-filtration 12 for the trapping of airborne particulates. The particulate pre-filtration 12 is preferably about 2 inches thick in one embodiment, and includes left and right pleated particulate pre-filters 14,16. The particulate pre-filters 14, 16 trap gross particulates (e.g., dust and bugs) from the outside air before that air reaches the other filtration media in the air purifier 2 discussed infra. Suitable filters for the particulate pre-filtration 12 are those having a Minimum Efficiency Reporting Value (“MERV”) of 5 to 13 with an Average ASHRAE Dust Spot Efficiency (Standard 52.1) of 20% to 80%, optionally from 20% to 95%. In certain embodiments, the upstream MERV is about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%. Particularly preferred filters for the particulate pre-filtration 12 are pleated filters having a MERV of 7 to 8, with an Average ASHRAE Dust Spot Efficiency (Standard 52.1) of 30% to 45%. In certain embodiments the filters may be, for example, HEPA filters. Alternatively, ULPA filters may be suitable.

Proper particulate pre-filter selection should be guided by the need to trap gross-particulates without unduly affecting the air flow rate through the air purifier 2. The particular type of particulate pre-filter(s) selected for particulate pre-filtration depends on various factors, including outside air quality. It is preferred that the particulate pre-filtration 12 is located immediately upstream from the additional filtration media discussed infra, as shown in FIGS. 3 and 4. Alternatively (or in addition), however, particulate pre-filtration may be located further upstream, e.g., in upstream ductwork of an HVAC system or AHU into which the air purifier 2 is installed.

Downstream from the particulate pre-filtration 12 is VOC pre-filtration 18. Once air passes through the particulate pre-filtration 12, the air is effectively free of gross particulate that would otherwise diminish the efficacy and useful life of the VOC pre-filtration 18. VOC pre-filtration ideally includes adsorption media, such as carbon, as well as oxidation media, such as potassium permanganate (“KMnO₄”) or a photocatalytic oxidizer. A particularly preferred type of carbon is virgin coconut shell. In a preferred embodiment, the VOC pre-filtration 18 is a carbon and KMnO₄ blend, e.g., in a 50/50 proportion. In some embodiments, the blend may include additional elements. The proportion of the blend may vary depending on the types and levels of VOCs present in the source air. Ideally, the source air would be tested for VOCs, and, based on test results, a custom blend would be prepared to maximize VOC removal in a given environment. In an alternative embodiment of the VOC pre-filtration (not shown), separate (i.e., non-blended) carbon and KMnO₄ filters are used.

The embodiment of the VOC pre-filtration 18 shown in FIGS. 3 and 4 includes a total of twenty stacked filter trays 20,22, whereby ten such trays 20 are on the left side of the housing 4 and ten such trays 22 are directly adjacent, to the right. The length of the trays, i.e., the longitudinal distance over which the air flows, is preferably about 17 inches in one embodiment, though it may be shorter or longer. Each tray 20, 22 includes two blended carbon and KMnO₄ filters 24, arranged in a V-bank along a vertical plane (e.g., the plane of FIG. 3). The V-bank arrangement increases the surface area of the filters 24 over which air must travel, thereby enhancing the effectiveness of the VOC pre-filtration 18. Once air passes through the VOC pre-filtration 18, the VOC load of the air is effectively reduced.

Downstream from the VOC pre-filtration 18 is particulate post-filtration 26 for the trapping of airborne particulate, e.g., particulate generated by the VOC pre-filtration 18 (such as carbon dusting). The particulate post-filtration 26 includes left and right pleated particulate post-filters 28, 30. The filters used in the particulate post-filtration 26 may be identical or similar to those used in the particulate pre-filtration 12, discussed supra. While particulate post filtration 26 downstream from the VOC pre-filtration 18 is preferred, it may not be necessary in all applications. For example, if the VOC pre-filtration is of a type that does not generate air-borne particulate, such as bonded carbon, particulate post-filtration may be optional.

Downstream from the particulate post-filtration 26 is UV filtration 32 which destroys airborne biological contaminants and, in some embodiments, degrades chemical contaminants. Whether or not particulate post-filtration 26 is used, the air reaching the UV filtration 32 should be effectively free of gross particulates and contain dramatically reduced levels of VOCs so as not to diminish the efficacy of the UV filtration 32.

The UV filtration may include one or more UV sources, although a plurality of UV sources is preferred. It is further preferred that these UV sources are UVC sources, capable of generating UV radiation at a wavelength varying from 220 nm to 288 nm. Most preferably, the UVC sources are capable of generating UV radiation at a wavelength of 260 nm, however commercially available UVC sources capable of generating UV radiation at a wavelength of 254 nm are adequate. In an alternative embodiment described in U.S. Pat. No. 5,833,740 (Brais), which is incorporated herein by reference in its entirety, the UV filtration includes at least one vacuum UV source, capable of generating UV radiation at a wavelength varying from 170 nm to 220 nm (preferably 185 nm) and at least one UVC source, capable of generating UV radiation at a wavelength varying from 220 nm to 288 nm (preferably 260 nm). In that embodiment, the UVC source is preferably downstream from the vacuum UV source. When operating, the vacuum UV source breaks oxygen molecules into mono-atomic oxygen which then reacts with chemical contaminants present in the air and then degrades them by successive oxidation to odorless and inoffensive byproducts. The UVC source kills biological contaminants present in the air by irradiation and degrades residual ozone produced by the vacuum UV source into molecular oxygen.

Particularly preferred UV filtration 32 shown in FIGS. 3 and 4 is the “UV Bio-wall” made by Sanuvox. Alternatively, the “Bio 30GX,” which is also made by Sanuvox, is a preferred type of UV filtration. The UV filtration 32 includes a pair of fixtures 34, 36 each of which has five UV lamps 38 (not all five of which are visible in the Figures). The UV lamps 38 are preferably about 60 inches long and extend longitudinally through the housing 4 so as to maximize exposure time of the air to UV radiation. In one embodiment, the UV lamps are UVC sources, providing UV radiation within the UVC wavelength parameters discussed supra. In an alternative embodiment, described in U.S. Pat. No. 5,833,740 (Brais), each lamp 38 is dual-zoned, having an upstream vacuum UV source and a downstream UVC source. In that alternative embodiment, the upstream vacuum UV source may, e.g., be a high intensity mercury vapor lamp capable of generating UV radiation having a wavelength in a range of about 170 nm to about 220 nm, and the downstream UVC source may, e.g., be a low intensity mercury vapor lamp capable of generating radiation having a wavelength in a range of about 220 nm to about 288 nm. The interior 44 of the housing 4 encasing the UV filtration 32 is highly reflective, with a preferable coefficient of reflection of at least 60%, so as to enhance the effectiveness of the lamps 38.

The kill rate of biological contaminants is a function of the intensity of UVC radiation produced by the UV filtration 32 and reflected by the interior 44 of the housing 4, as well as the exposure time of such contaminants to the UVC radiation. Thus, the higher the intensity of the UVC radiation and the longer the exposure time of such contaminants to the UVC radiation, the greater is the level of sterilization achieved. Depending on factors such as the desired level of sterilization, the amount of space available to house UV filtration, and costs of operating and maintaining UV filtration, the desired total UVC output of the UV filtration 32 may vary. In one actual embodiment, it was found that a total UVC output ranging from about 33,464 μJ/cm² to about 90,165 μJ/cm², with an average total UVC output of about 43,771 μJ/cm², provided a desired level of sterilization, given practical constraints of cost and space. Such total UVC output killed 100% of numerous biological contaminants including, but not limited to smallpox, flu, tuberculosis, anthrax and H1N1 virus.

The UV filtration 32 contained within the housing 4 is likely not visible to a user of the air purifier 2 when in use, because direct UV exposure is harmful to humans. Thus, a user cannot ascertain visually (i.e., by simply looking at the air purifier 2 itself) whether the lamps 38 are operating at a given time. It cannot be assumed that the air purifier 2 is effectively destroying air-borne biological and chemical contaminants, without knowing for sure that the UV filtration is operating properly. Accordingly, it is preferred that the present disclosure include sensors and a monitor (not shown) to detect and indicate, respectively, how much time each UV lamp 38 has been in use and whether each lamp 38 is operating at a given time. The monitor may include, e.g., a scrolling digital clock, which indicates the length of time each lamp 38 has been operating. These sensors and monitor would indicate to a user when it is time to replace any of the lamps 38.

As a general matter, moisture within the housing 4 can foster the growth of biological contaminants. Accordingly, it is preferable to include a UVC source in the vicinity of areas in which moisture is generated or gathers. For example, upstream from the particulate pre-filtration 12 may be one or more cooling coils (not shown) that help to ensure that the air which is treated by the air purifier 2 is moderate in terms of temperature. Such cooling coils tend to generate moisture. It is therefore preferable to include a UVC source adjacent to such cooling coils. Similarly, it may be appropriate to include a UVC source immediately upstream from a filter/diffuser (not shown) from which the air enters into a substantially enclosed space, e.g., an IVF laboratory or other room, after leaving the air purifier 2.

Downstream from the UV filtration 32 is VOC post-filtration 46, which capture, e.g., VOC by-products of the irradiation from the UV filtration 32. Possible embodiments of the VOC post-filtration 46 include any of those discussed supra regarding the VOC pre-filtration 18. The VOC post-filtration 46 shown in FIGS. 3 and 4 includes left and right VOC post-filters 48, 50 that are arranged in a V-bank along a horizontal plane (e.g., the plane of FIG. 4). The VOC post-filters 48, 50, like their upstream counterparts, are preferably blended carbon and KMnO₄. Although VOC post-filtration 46 is preferred, in some applications, it may not be required and may thus be omitted. In certain embodiments, the VOC post-filtration is omitted.

Gametes and the human embryo are highly sensitive to VOCs, even in amounts considered negligible in other applications. Thus, in a preferred embodiment, the VOC filtration (both pre-filtration 18 and/or post-filtration 46) operates effectively to remove VOCs from air that is fed into an environment in which WF is being conducted or to remove VOCs from air that is recirculated through the air purifier in an environment in which WF is being conducted. Accordingly, one or more sensors for detecting VOC levels (not shown), preferably in real time, may be placed in an IVF laboratory and coupled to a monitor (not shown) to indicate the VOC levels in the laboratory at a given time. In optional embodiments, VOC sensors may be placed upstream of the air purifier, downstream of the air purifier or both upstream and downstream of the air purifier. With such in-room VOC detection, a user of the air purifier 2 would know when it is time to replace the VOC pre-filtration 18 and post filtration 46, and/or whether an alternative type or blend of VOC filters would be more suitable. While in-room VOC detection is particularly useful in an WF laboratory, it may be helpful in any environment requiring low VOC levels.

Downstream from the VOC post-filtration 46 is final particulate filtration 52, which traps substantially all remaining particulate in the air before the air exits the outlet 8. Final particulate filtration 52 preferably includes one or more filters capable of trapping fine airborne particulate, e.g., filters having a MERV of 13 or greater with an average ASHRAE Dust Spot Efficiency (Std. 52.1) of 80% or greater. More preferably, such filters have a MERV of 16 or greater with an average ASHRAE Dust Spot Efficiency (Std. 52.1) of 95% or greater. Most preferably, such filters have a MERV of 17 or greater with an average ASHRAE Dust Spot Efficiency (Std. 52.1) of 99.97%, as do HEPA filters. Alternatively, ULPA filters may be suitable. The choice of filter(s) for final particulate filtration should be guided by the potentially competing needs of maintaining an optimal air flow rate and effectively removing particulate from the air.

The final particulate filtration 52 of FIGS. 3 and 4 includes left and right 12-inch thick HEPA filters 54, 56. Preferably, magnehelic gauges (not shown) are placed both upstream and downstream from the HEPA filters 54, 56 to measure the pressure drop across those filters. The degree of pressure drop will assist in the identification of the proper time in which to change the HEPA filters 54, 56, or other filters used for final particulate filtration.

Downstream from the final particulate filtration 52, is an atomizing humidifier 58. The humidifier 58 may or may not be necessary, depending on the needs of the facility in which the air purifier 2 is being used. If a humidifier 52 is needed, it may be placed downstream from the final particulate filtration 52 so that the moisture does not adversely affect the performance of the VOC post-filters 48, 50, the HEPA filters 54, 56, or other filters used for final particulate filtration. In alternative embodiments, the humidifier may be placed upstream of the air purifier system of the disclosure. In alternative embodiments, the humidity of the air entering the air purification system of the disclosure is controlled. Humidified air can contain and support the growth of biological contaminants. Accordingly, if a humidifier 58 is used, an additional UVC source (not shown) to destroy such contaminants may also be included. This additional UVC source may be downstream from the humidifier 58, preferably at the last point in ductwork before entry into a room served by the purified air.

In exemplary embodiments, the air purifier system of the disclosure may comprise sequences and types of air filtration media selected from any one or all of the following, in any order: pre-filtration, HEPA filters, particulate filtration, VOC filtration, post-VOC filtration, UV filtration which may include one or more UV sources, and particulate post filtration. In certain embodiments, the VOC filtration comprises one or more filters containing media selected from the group consisting of blended carbon, KMnO₄, and combinations thereof. In certain embodiments, the particulate filtration includes one or more filters selected from the group consisting of HEPA filters and ULPA filters. In certain embodiments the UV filtration includes at least one UV source. In certain embodiments the UV filtration includes a plurality of UV sources. In certain embodiments the UV filtration includes at least one UVC source, capable of generating UV radiation at a wavelength of from 220 nm to 288 nm. In certain embodiments the UV filtration includes at least one UVC source capable of generating UV radiation at a wavelength of 254 or 260 nm. In optional embodiments, VOC sensors may be placed upstream of the air purifier, downstream of the air purifier or both upstream and downstream of the air purifier. In certain embodiments, the air purifier is a stand-alone unit which purifies in—room air. In certain embodiments, the air purifier is an in-room unit that purifies in-room air. In certain embodiments, the source air that is to be treated by the air purifier has a temperature of between about 50° F. to about 72° F. and/or a humidity of between about 30% to about 70% relative humidity. In exemplary embodiments, the source air that is to be treated by the air purifier has a temperature of between about 68° F. and about 75° F. and a humidity of between about 45% and about 55% relative humidity. In an exemplary embodiment, the source air is recirculated in-room air.

Accordingly, another aspect of the present disclosure includes purified air, such as that attainable using an air purifier as described herein. Ideally, such purified air would be characterized by a high level of purity as measured by any one or more of three parameters: (a) “TVOC,” i.e., total volatile organic compounds, measured in “ppb,” or parts per billion; (b) “Biologicals,” i.e., biological contaminants, including spores, measured in “CFU/M³,” or colony forming units per cubic meter; and/or (c) “Particulate,” i.e., the number of particles per cubic foot having, e.g., nominal sizes of 0.3 μm or 0.5 μm.

TVOC measurements may be made, e.g., using GRAYWOLF SENSING SOLUTIONS, Model No. TG-502 Toxic Gas Probe with Photo Ionization Detector (“PID”) sensors utilizing a 10.6 eV lamp calibrated to Isobutylene. The lowest detectable limit of TVOCs using the TG-502 Toxic Gas Probe is 5 ppb.

To ensure accuracy, measurements of Biologicals are preferably assessed using two complementary methods. According to a first method of measuring Biologicals, ambient air (i.e., the air being tested) is drawn over ALLERGENCO D spore traps using a high volume vacuum pump calibrated to draw 15 liters of air per minute. This is done for 10 minutes, so that a total of 150 liters of air is drawn through the spore trap cassette. The traps are then examined by direct light microscopic observation to determine the identification of some select types of biological contaminants present in terms of CFU/M³. According to a second method of measuring Biologicals, an ANDERSON N6 sampler is utilized to obtain culturable air samples (from the ambient air being tested) on three types of media: malt extract agar, cellulose agar and DG-18. The sampler is calibrated pre- and post-collection to draw a rate of 28.3 liters per minute for a sample time of 5 minutes. Using this second method of measuring Biologicals enables determination of the unique identification of any biological contaminant present in terms of CFU/M³ due to the three different types of growth media.

The particulate measurements may be made, e.g., using a TSI AEROTRAK 9306 Handheld Particle Counter. The particle counter is preferably calibrated with NIST traceable PSL spheres using TSI's Classifier and Condensation Particle Counters, the recognized standard for particle measurements. The particle concentrations in the air are measured at nominal particle sizes of 0.3 μm, 0.5 μm, 1.0 μm, 3.0 μm, 5.0 μm, and 10.0 μm, per cubic foot (ft³).

In a preferred embodiment, it is contemplated that purified air attainable using an air purifier as described herein, is characterized by any one or more of the following parameters: (a) a TVOC content of less than 5 ppb (or below detectable limits using the GRAYWOLF SENSING SOLUTIONS, Model No. TG-502 Toxic Gas Probe with PID sensors described supra, or another instrument with similar measurement capabilities and tolerances); (b) a Biologicals content of less than 1 CFU/M³ (or below detectable limits using the methods of measuring Biologicals described supra, or other methods with similar measurement capabilities and tolerances); (c) a particulate content of from about 1,000 0.3 μm particles per ft³ of air to about 10,500 0.3 μm particles per ft³ of air, or from about 600 0.5 μm particles per ft³ of air to about 1,000 0.5 μm particles per ft³ of air, and (d) combinations thereof.

Depending on the application or environment, acceptable levels of TVOCs, Biologicals and particulates may vary. For example, in one embodiment, the purified air may be characterized by any one or more of the following parameters: (a) a TVOC content of from less than 5 ppb to about 500 ppb; (b) a Biologicals content of from less than 1 CFU/M³ to 150 CFU/M³; (c) a particulate content of from about 1,000 0.3 μm particles per ft³ of air to about 50,000 0.3 μm particles per ft³ of air, or from about 600 0.5 μm particles per ft³ of air to about 500,000 0.5 μm particles per ft³ of air, and (d) combinations thereof. More preferable particulate content is from about 1,000 0.3 μm particles per ft³ of air to about 30,000 0.3 μm particles per ft³ of air, or from about 600 0.5 μm particles per ft³ of air to about 10,000 0.5 μm particles per ft³ of air. Particularly preferred particulate content is from about 1,000 0.3 μm particles per ft³ of air to about 10,500 0.3 μm particles per ft³ of air, or from about 600 0.5 μm particles per ft³ of air to about 1,000 0.5 μm particles per ft³ of air.

In certain embodiments the purified air may have characteristics selected from the group consisting of: i) a TVOC content of from less than 5 ppb to about 500 ppb; ii) a Biologicals content of from less than 1 CFU/M³ to 150 CFU/M³; and combination thereof.

In certain embodiments the purified air may have characteristics selected from the group consisting of be characterized by: i) a TVOC content of from less than 5 ppb to about 500 ppb; ii) a particulate content of from about 1,000 0.3 μm particles per ft³ of air to about 50,000 0.3 μm particles per ft³ of air, or from about 600 0.5 μm particles per ft³ of air to about 500,000 0.5 μm particles per ft³ of air; and combinations thereof.

In certain embodiments the purified air may have characteristics selected from the group consisting of: i) a Biologicals content of from less than 1 CFU/M³ to 150 CFU/M³; and/or ii) a particulate content of from about 1,000 0.3 μm particles per ft³ of air to about 50,000 0.3 μm particles per ft³ of air, or from about 600 0.5 μm particles per ft³ of air to about 500,000 0.5 μm particles per ft³ of air; and combinations thereof.

The disclosure will be illustrated in more detail with reference to the following Examples, but it should be understood that the present disclosure is not deemed to be limited thereto.

### EXAMPLES

### Example 1. Comprehensively Examine Air Quality with and without the Installation of a Transformational Air Purification System Intervention

### Ambient Air Assessment

Air quality testing will be performed to appraise particulates, biologicals and VOCs in areas with an existing air filtration system and all other means of environmental sterilization occur, for 6 months prior to the installation of the APS. The air quality will be appraised in both the control group unit and the experimental group unit settings for this six month window. Following the installation of the APS, the ambient air quality will be appraised for a 30-month period, concurrent with the project timeline. The air quality will be appraised in both the control group and experimental group following the installation of the APS in the experimental group HVAC system, at 24 nodes or locations—which breaks down to 12 unique nodes/locations for both the control group's floor and the experimental group's floor. There will be nodes identified in each of the various resident settings, including resident rooms, hallways, bathrooms, nursing stations, and etc. All 24 nodes on a building plan, so as to always test in the same location through testing, and establish a testing schedule in collaboration with the medical professionals at LTCF. The testing schedule for testing at all nodes is as follows; real-time total VOC concentrations by photoionization detection (4 times/month), quantitative EPA TO-15 VOC, and EPA TO-11 (aldehydes) canister testing (2 times/year), Particle Counting (4 times/month), biologicals (1 time/month per method—4 biological methods per month), and surface assessment (1 time/year). In the event of a viral or bacterial outbreak, the team will opt for more frequent biological testing. The air quality will be appraised for particulates, VOCs, and pathogens as detailed herein. The methods are detailed below, followed by the general methodology for our air sampling protocol.

### General Methodology for Air Sampling

The detailed protocol for air sampling in the LTCF will be followed. The research team will select and procure an appropriate handheld sampling apparatus, air sampler, or growth media in plates for the selected air contaminant to be quantified. The researchers will identify and record the specific location of test. If using a handheld air quality appraisal method, the researchers will record the node and reading in the laboratory notebook. If using an air sampler, it should preferably be placed on the floor, in the center, sides, or corners of the room. If using an air sampler, install the petri dish, filter, or other media in the air sampler. Operate the air sample for the time period specified by the manufacturer instructions for the specific contaminant After the elapsed time period, shut down the air sampler and carefully remove the plate (or filter). The research team will label the plate with an appropriate node or description of the location, the type of sample (Bacteria or Fungi), and date as necessary. Record the sample information on a separate log of all samples. The team will record room occupancy, including brief entrances or exits. If required, the researchers will deliver the sample to the laboratory. If samples have been delivered to a laboratory then the results will be directly inspected. The plate counts should be tabulated with each sample taken and averaged for each specific location. The research team will take digital images of the plates for the record, the digital images may be used for counting and the plates disposed.

### Specific Methods for Air Quality Assessment (Particulates, VOCs, Pathogens/Biologicals) Particulates

A TSI AEROTRAK 9306 particle counter, calibrated with NIST traceable PSL spheres using TSI's Classifier and Condensation Particle Counters (recognized standard for particle measurements), will be used to log six particle sizes. This hand held particle counter is only 1.0 kg in weight and complies with all requirements of ISO 21501-4. The particle counter utilizes a laser diode to detect particles between 0.3-25 □μm. This detection range provides detection to almost an order of magnitude below PM2.5, the fine particulate classification. The battery life provides more than 6 hours of continuous use for the research team. Data will be presented as the differential particle count (the concentration between each particle size range) per cubic foot of air over the sampling period. This particulate count provides the research team with detailed appraisal of the total number of airborne particulates, as well as general size characterization of these particulates. It is expected that the research team will see higher levels of particulates in the control group unit tests. This real-time particulate appraisal will occur four times per month.

### Volatile Organic Compounds (Airborne Chemical)

VOCS will be appraised using EPA TO-15 (all VOCS), EPA TO-11 (aldehydes) and Photo Ionization Detector (PID) (TVOCS) for real time monitoring of individual and total volatile organic compounds. The three methods of assessment will evaluate the presence of parts per billion (ppb) levels of volatile organic compounds in the area collected during a four-hour period. Blank canisters will be used as negative controls during EPA TO-15 and TO-11 testing. The data will provide an accurate assessment of the environmental load of individual and total VOCs at the selected nodes. It is anticipated that the research team will see higher levels of VOCs in the control group unit tests, and lower VOC levels in the experimental group unit.

The U.S. EPA method TO-15 and TO-11 provides a standardized method to analyze indoor VOCs and aldehydes. The method provides guidance on the deployment of specially designed stainless steel canisters. The specially designed steel canisters are designed to be deployed under vacuum, and they slowly infill due to regulators, during the designed 24-hour testing window. The stainless steel canisters then seal, which provides sample stability during transport to the lab. The EPA method also provides specific guidance of sample analysis via gas-chromatography-mass spectrometry (GC-MS). A contract laboratory may be used for U.S. EPA method TO-15, as this allows the added benefit of data using an EPA accredited laboratory. The EPA accredited laboratory will use GC-MS to detect the unique volatile and semi-volatile compounds. The method proposed will analyze for 61 TICs (61 common “Tentatively Identified Compounds”). In addition to the analysis of the 61 TICs, there is a database search performed by software coupled to the GC-MS system which searches nearly 100,000 compounds for a statistically viable match. As an example of detection limits, some likely VOCs in healthcare settings and their detection limits are provided: acetone 25 ppbv, benzene 5.0 ppbv, chloroform 5.0 ppbv, isopropyl alcohol 25 ppbv, styrene 5.0 ppbv, toluene 5.0 ppbv. Following EPA method TO-15 and TO-11, the research team will have detailed VOC concentrations, specific to each compound and part per billion (ppb) level analysis. This detailed VOC analysis will occur twice per year.

In order for the team to appraise real-time total VOC (TVOC) in the LTCF, the team will utilize multiple hand-held parts per billion VOC monitors. The team will use the ppbRAE parts per billion VOC Monitor (Model PGM-7240), which is a portable ion detector (PID) for real-time VOC testing. This handheld ppbRAE is only 19.5 ounces and is easily deployable for up to 10 hours of continuous monitoring before recharging the batteries. The lamp and sensor are calibrated prior to use to enable sensitivity of only a few ppb. While this robust sensing device provides real-time VOC concentrations, when monitoring indoor air quality, it must be pointed out that there is no characterization/speciation of all individual VOC. The real-time VOC testing only provides total VOC concentrations. This real-time TVOC data can be compared with the U.S. EPA TO-15 and TO-11 methods, to provide the research team with insight on total VOC loading and specific compound loadings that are detected during the study. This real-time VOC appraisal will occur 4 times per month.

### Airborne Pathogens/Biologicals

To employ the most robust and sensitive means to assess the presence of environmental biologicals, four tests will be employed. In general, there are two ‘standard’ methods to appraise biologics in air, active air samplers and settle plates. In order to provide the most robust air quality appraisal, the research team aims to use both active methods and settle plates. Biological data identifies the organism and its concentration. Bacterial counts are quantified by the number of colony forming units per cubic meter of air (cfu/m³). Viral counts are quantified by the number of viruses per cubic meter of air. Fungal counts are quantified by the number of spores per cubic meter of air. Sampling also includes settle plate sampling to identify each agent. Surface and duct assessments will attempt to identify the type and quantity of organism or contaminant found but may be limited by the sampling size. The first three methods, will follow the General Air Sampling Protocol detailed above, while the fourth method (settle plates) will follow the specific Protocol for Settle Plate Sampling provided below.

1. Ambient air will be drawn over Allergenco D spore traps using a high volume vacuum pump calibrated to draw 15 liters of air per minute, for 10 minutes, so that a total of 150 liters of air is drawn through the spore trap cassette. The traps will be examined by direct light microscopic observation to determine the unique identification of each biological present in spores/cubic meter.
2. An Anderson N6 sampler will be utilized to obtain culturable air samples on three types of media (malt extract agar, cellulose agar and DG-18). The sampling vehicle will be calibrated pre- and post-collection and will draw a low rate of 28.3 liters per minute for a sample time of 5 minutes. The testing mechanism will be able to determine the unique identification of each biological present in colony forming units/cubic meter.
3. An air sampler coupled with PCR technology will assess the presence and concentration of viruses (i.e. influenza, measles, adenovirus, coxsackievirus, parainfluenza, rotavirus, coronavirus, rhinovirus and RSV) within the environment in colony forming units/cubic meter.
4. The first three tests provide quantitative biological testing. Plate sampling will be used to qualitatively identify pathogens. Triplicate plates should be used at each location in the area. The settle plate media should be selected carefully so as to detect both gram-negative (e.g. vancomycin-resistant enterococci (VRE) and gram-positive bacteria (e.g. Staphylococcus aureus).

### General Method for Plate Sampling

The following test protocol will be used when the settle plate method is used for sampling hospital air for bacteria and/or fungi.

### Preliminary Conditions and Materials

The research team will select and procure appropriate growth media in plates suitable for the intended application: Bacteria and Fungi. For comparative testing three plates will be used at each node. The team will keep one to three plates unused as controls. The team will keep room conditions within normal indoor operating temperature and humidity ranges. Test locations will be selected and will preferably include node/room floor locations at the center, the side, and the corner. The team will ensure that occupancy can be kept as normal or unoccupied rooms can be tested for baseline conditions. The team will number/identify the individual plates before they are placed at test locations. To reiterate, a minimum of three plates (bacteria and/or fungi) is recommended for each test location for comparative testing.

### Test Protocol

Upon entering the node/room, the team will close any window blinds if there is direct sunlight in the room. Then, place the settle plates at the designated locations in the room. Each three-plate set of plates may be placed side by side, at each test location. The team will record each test location and the time of placement. Other notes to be taken by the team include, room occupancy, including brief entrances or exits. After a period of 1-2 hours, the team will cover and remove the settle plates. The team will make sure the plates are labeled or numbered before or at the time they are removed, and record the removal time. The team will deliver them to the laboratory for incubation and counting. Repeat the above procedure for fungi (or bacteria) if necessary. Fungi and bacteria may be tested coincidentally, with six plates placed side by side at each test location.

### Evaluation of Results

The team will deliver to the laboratory for culturing. The team will count the colonies on the plates and record them in their laboratory notebooks. Then, the team will evaluate the counts and determine the mean counts of the settle plates as compared with the control plates. Based on the results, the team will identify the samples and identify any hazardous pathogens. The team will identify all species and record this information. The appearance of dangerous or unusual pathogens will be brought to the attention of LTCF and/or medical authorities (i.e., CDC), if necessary.

### Surface Assessment

In order to comprehensively appraise the environment, the research team will also perform surface sampling will provide qualitative data in terms of contaminating species but will not provide quantitative data due to the absence of absolute references for levels of surface contamination. A large percentage of surface fomites originate from airborne pathogens. Surface sampling will assess the relative contribution of airborne pathogens to the common surfaces such as floors and walls, doorknobs, faucet handles, medical equipment, etc. In addition, the research team will perform surface assessment on ductwork and HVAC final diffusers. The ATP swabbing of surfaces is detailed below.

### General Method for Surface Sampling

The following procedure will be used for surface sampling, including cooling coils. The same procedure will be used to test prior to installation of the inventive system and for each test sample after installation. The research team will procure surface sampling materials as necessary, which include sterile swabs (wet or dry), sterile gloves, and petri dishes. At least 3 plates (or swabs) will be needed for each condition. The researchers will utilize gloves sterilized through the use of a disinfectant such as alcohol. If a sterile template is used to mask off a surface area, then the template must be discarded after each use. The team will keep at least one plate unused as a control for each day of testing.

The research team will first disengage any UVGI systems and any operating equipment in the vicinity that may be a hazard to test personnel. If entry into an AHU is required, the fan should be shut down. The research team will identify and record the location of a suitable surface sampling point within a node/room. The sample location will be approximately the same for subsequent samples. The research team will visually estimate an area of approximately 2 inches² and will use the wet or dry swab, draw it gently across the sample area with a back and forth motion, and either insert the swab back into the sterile container or draw the swab across the petri dish. The research team will cover the plate, seal as necessary, and label the swabs and/or the plates with a node description of the location sampled. The team will repeat this process for all desired nodes. The swabs will be delivered to the laboratory. Digital images of the plates will be made and used for counting. The team will tabulate and summarize the results as necessary and record the data in the laboratory books.

### Clinical Data

All methods of reporting and analyzing clinical data are detailed to fulfill Aim 2, which is:

### Aim 2. Establish the Impact of Comprehensive Remediation of Airborne Environmental Risks on Senior Health Based on Patient Outcomes in a Long-Term Care Facility.

### Overview of Clinical Data Collection

The research team will keep all clinical and demographic data will be anonymous. These data will include gender, race, reason for admission (symptoms), duration of stay in hospital, presence of any HAI, medication use, and patient outcome. The data collection will begin with retrospective data for the year prior to commencement of the study will be collected in the same categories for both the control group and the experimental group. Following the installation of the Air Purification System, prospective data will be collected on a monthly basis for a 30-month period from commencement of the study by independent data collection personnel, entered onto the data collection form and transposed to the database. All entered data will be audited and validated for accuracy before being included in the dataset.

### Clinical Data Analysis

Analyses will be focused on the environmental impact of the air purification systems and methods of the disclosure based on environmental testing as detailed in the section, “Testing of the Environment.” The most direct analyses will involve the comparison of contaminant levels pre-installation and post-installation to quantify the impact of the filtration system on the levels of airborne contaminants in the selected clinical areas. Ongoing analyses will assess the levels of contaminants throughout the 30-month testing period. Clinical analysis will use the monthly data collected as outlined above. The most direct analyses will provide a comparison of pre- and post-installation HAI rates. These data will be further analyzed to determine if there are any other factors that may have caused a decline or no change in HAI such as demographic factors, change in procedures, etc. In addition, the team will seek to understand any correlations between other patient outcomes available in the PHC4 data. The research team will seek to develop interim reports that will be generated quarterly during the study for all partners to review and summarized in annual reports. The annual and final reports will be presented to the LTCF for comments prior to being finalized. The annual reports and final report will become the basis of articles that would be submitted for publication.

### Data Collection, Maintenance and Analysis

All data will be collected following strict HIPAA guidelines and only with the permission and approval of the LTCF. Data will be entered into a computerized database utilizing FilePro version 12 for Mac. This is a relational database that will allow linkage of multiple datasets using key ID fields such as LTCF combined ID. These data sets will be password protected and follow all data security protocol requirements. There are four specific datasets that will be developed through this work, and these include:
(1) LTCF reference site data
(2) Environmental sampling results.
(3) Occupancy rates and characteristics of occupants in Control Group and Experimental Group.
(4) Retrospective and prospective HAI data.

### Example 2. Comprehensive and Live Air Purification as a Key Environmental, Clinical, and Patient Safety Factor

Healthcare organizations strive to provide optimal patient experience by improving the quality of patient care, enhancing clinical outcomes, while at the same time containing associated costs. In the United States, the CDC has estimated that more than 1.7 million people suffer from an infectious complication within the hospital environment annually, representing between 5-10% of all admissions and annual costs ranging between $35B and $88B. It has been demonstrated that the majority of infectious surface fomites originate from the air. Consequently, a reduction in airborne bacterial and fungal pathogens should be associated with a reduction in surface fomites. This CDC study represents the first comprehensive evaluation of infectious and aerosolized pathogens and their speciation, location and concentration within a typical hospital setting. The study provides important data regarding the complex relationship between airborne pathogens and air filtration methodologies in the context of the molecular and microbial epidemiology of illness and infections in the clinical setting. The results from the study demonstrated that use of a transformational air purification system as provided herein provides comprehensive remediation of airborne pathogens and a significant reduction in surface oriented infectious fomites. Overall reduction of airborne and surface bacterial and fungal pathogens responsible for patient illness and infections will result in a reduction of associated illnesses, HAI rates, and improved metrics of patient care inclusive of, but not limited to, length of stay and readmission rates. Improvements in these outcome metrics should, by association, correlate to risk mitigation and cost avoidance.

Historically, it has been understood that the patient, the health-care worker, and various surface areas collectively constitute the primary repositories of pathogens responsible for majority of HAIs [O'Connor, C., The impact of healthcare-associated infections on patient care and the role of diagnostic molecular technology in infection prevention and control practice. 2016]. To that end, infection control protocols, in-room sterilization techniques, patient preparation and hand-washing protocols have been implemented in most hospitals and have been helpful in reducing overall HAI rates [Sharpe, P. A. and M. G. Schmidt, Control and mitigation of healthcare-acquired infections: designing clinical trials to evaluate new materials and technologies. HERD: Health Environments Research & Design Journal, 2011. 5(1): p. 94-1151. Moreover, recent literature suggests that a significant proportion of pathogens responsible for HAIs are airborne [KOWALSKI, W. J., Hospital-Acquired Infections. HPAC Engineering, 2007]. The advanced air purification technology as disclosed herein, as shown in U.S. Pat. Nos. 8,252,099; 8,252,100; 9,522,210; 9,675,725; and 9,980,748, incorporated by reference in their entireties, and as exemplified in FIGS. 1-4 herein, and sold under the trademark Aire˜HCX™ (LifeAire Systems, Allentown, Pa.), was specifically designed to comprehensively address these airborne pathogens (AP) as they are inherently generated during routine clinical operations. In environments where an embodiment of the air purifier described herein is employed, infectious APs are remediated in both the supply and return air before entering the clinical space. The advanced air purification technology as disclosed herein exceeds the limitations associated with commonly utilized mechanisms of air filtration.

Many in-room sterilization technologies require that the clinical space be vacated before use, leading to temporary loss of functional space. The in-room approaches also provide a “static” clean at the exact time of use. Reentry of patients and health-care workers and the initiation of clinical processes inherently serve as a source of rapid reestablishment of pathogen populations. Unlike the “static” clean model of many of the in-room units, the advanced air purification system as disclosed herein (LifeAire Air Purification System, “LAS-APS”) provides real-time remediation of APs as they are generated during clinical operation. In addition, unlike the “capture” model of the commonly used HEPA filters, the LAS-APS uses a “kill” model and is mathematically and genomically modeled to destroy the DNA and RNA of all bacteria and viruses such that they are rendered non-infectious. HEPA filtration is based upon the capture of viable biological particulates, allowing the spores to grow and proliferate above the space being protected. Air flowing over the spores can disturb and dislodge them such that they enter the clinical space [Price, D. L., et al., Mold colonization during use of preservative-treated and untreated airfilters, including HEPA filters from hospitals and commercial locations over an 8-year period (1996-2003). Journal of Industrial Microbiology and Biotechnology, 2005. 32(7): p. 319-321]. The “kill” mechanisms incorporated into the LifeAire Systems' technology eliminate these possibilities.

The advanced air purification system as disclosed herein is installed within the healthcare facility's HVAC ductwork. The system is designed to deliver ultra-pure, contaminant-free air to any clinical environment. Based on over a decade of research, development, and testing, the air purification system as disclosed herein has been tested and proven to deliver air that is 99.99% free of any contaminants, with an associated air purity guarantee. In essence, the air purification system as disclosed herein was designed to remove all airborne biological pathogens and thus enhance patient safety of the intended health-care environment by reducing HAI incidence rates. With 69%-80% of the pathogens responsible for HAIs being airborne at some point, aggressive remediation of all airborne pathogens will provide for improved patient care and outcomes while reducing the financial burden associated with HAIs.

The advanced air purification system as disclosed herein LAS-APS provides extremely high levels of filtration as it was designed to kill the anthrax spore (e.g., the most difficult biological pathogen to kill) [Banerjee, D., et al., “Green” oxidation catalysis for rapid deactivation of bacterial spores. Angewandte Chemie International Edition, 2006. 45(24): p. 3974-3977]. The technology used in the air purification system as disclosed herein has been tested by the National Homeland Security Research Center and by other third parties. Results indicate that the system renders a broad-spectrum of pathogens inert and that it virtually eliminates threatening biological pathogens and volatile organic chemicals from the air—to a level of effectiveness not previously commercially available. Because of its effectiveness towards Bacillus anthracis, the air purification system as disclosed herein is able to remediate airborne pathogens such as Clostridium difficile, Aspergillus, Streptococcus, Pseudomonas, Staphylococcus (including methicillin-resistant variety), smallpox virus, Mycobacterium tuberculosis, influenza virus, etc., each representing a consistent threat to both the hospital environment and rates of HAIs.

### Materials and Methods

The design includes three zones within two medical surgical floors of a hospital campus. The three geographic zones include a control floor with air handling unit (“CF-AHU”) HEPA-filtration remediation (“AHU HEPA”), a zone with mixed AHU HEPA and the air purification system as disclosed herein (“MIXED”) with recirculated air, and a zone with the comprehensive air purification system as disclosed herein. Within each of these zones, two occupied and active patient rooms were selected for air quality testing. Each of two rooms was comprehensively evaluated per zone. The patient rooms were chosen such that the effects of elevators/entrances/exits as well as zone barriers would be minimized Rooms were further chosen to optimize direct comparisons of resulting data. Table 1 illustrates the zones and rooms evaluated during the study.

TABLE 1
HVAC design by study zone

| Zone | HVAC Design |
|---|---|
| CF-AHU | AHU HEPA Remediation |
| MIXED | AHU HEPA and LifeAire Systems Remediation |
| LSAR | LifeAire Systems Air Remediation |

During each testing event, one of the two rooms listed were chosen for the complete suite of particulate, biological and VOC testing. For this study, the two rooms in each zone were considered equivalent. For each specific testing event, room preference was for patient occupancy, followed by consistency within each room between measurements.

Each of the rooms underwent comprehensive evaluation for airborne and surface viable bacterial, fungal and VOC loads. Three commonly touched patient surfaces and two commonly touched clinical surfaces were evaluated per testing assay (Tables 2 and 3). In addition, the final diffuser providing supply air to the patient room and the return vents were swabbed for viable bacteria and fungi (Table 4).

TABLE 2
Patient Surface Sampling Sites

Bedside Table (Directly in front of patient)
IV Support Pole/IV Support Pole
Patient Remote Control - Number Buttons

TABLE 3
Clinical Surface Sampling Sites

IV Control Faceplate
Pressure Cuff Bulb

TABLE 4
HVAC Surface Sampling Sites

HVAC Room Diffuser
HVAC Room Return

### Testing Assay: Viable Bacteria By Air

Air testing was completed using the third-party laboratories, EMSL and Galson Laboratories under their proprietary method MICRO-SOP-132 [AIHA. AIHA Laboratory Accreditation Programs, LLC. 2018 Nov. 12, 2018]; Available from: www.emsl.com/PDFDocuments/Qualification/AIHA %20EMLAP %20-%20Houston.pdf.]. Following the standard operating procedures (SOPs) provided by the third party laboratories and using a Viable Andersen Cascade Impactor and calibrated pump, samples were gathered for 5 minutes at 28 liters per minute onto a soy agar plate. The five most concentrated species were then identified and quantified.

Testing Assay: Viable Bacteria By Swab Surface testing was conducted following all SOPs of the third party laboratories. Using a sterile swab, an area measuring 2-by-2 inch was sampled in each location with a smooth back and forth motion while rolling the swab for 10 seconds. The swab was then capped and sent to the third party laboratory for testing under method MICRO-SOP-132 [AMA. AIHA Laboratory Accreditation Programs, LLC. 2018 Nov. 12, 2018]; Available from: www.emsl.com/PDFDocuments/Qualification/AIHA %20EMLAP %20-%20Houston.pdf.]. The most prominent 5 types of bacteria were identified and quantified.

### Testing Assay: Viable Fungi by Air

Air testing was completed using a third-party laboratory under their proprietary method MICRO-SOP-202. Following the SOPs provided by the third party laboratories and using a Viable Andersen Cascade Impactor and calibrated pump, samples were gathered for 5 minutes at 28 liters per minute onto a MEA agar plate. The five most concentrated species were identified and quantified.

### Testing Assay: Viable Fungi by Swab

Surface testing was conducted following applicable SOPs of the third party laboratories. Using a sterile swab, an area measuring 2-by-2 inches was sampled in each location with a smooth back-and-forth motion while rolling the swab for 10 seconds. The swab was then capped and sent to the third party laboratory for testing under method MICRO-SOP-202 [30]. The 5 most prominent species of viable fungi were identified and quantified.

### Testing Assay: Volatile Organic Compounds (VOC) Testing

The measured VOC load of each room was determined using the methodology described in EPA TO-15. Using an evacuated container, air was captured for 15 minutes. The TO-15 assay determines VOCs in air collected using specially prepared stainless steel canisters and subsequently analyzed by gas chromatography/mass spectrometry (GC/MS). Due to the live hospital setting and available locations to place the testing cylinder, longer sampling times were considered but not employed due to the risk of sample tampering by unmonitored patients, visitors, and clinical staff.

### Testing Assay: Non-Viable Particulate Testing

Particulate testing was conducted using a modified NIOSH 0500 method. Sampling was conducted for 5 minutes at each testing site. The environmental testing was completed each month with sampling beginning in the morning and progressing through early afternoon. Clinical, housekeeping, operational staff and patients were blinded to both the study and zone locations to minimize any biases associated with behaviors or perceptions. Cleaning SOPs, patient care operations, patient appointment schedules, visitation, patient dining, and all operations of the floor remained unchanged. Sampling occurred during normal visitations, staff consultations, and meals to allow data acquisition and flow to simulate full hospital operations.

### Results

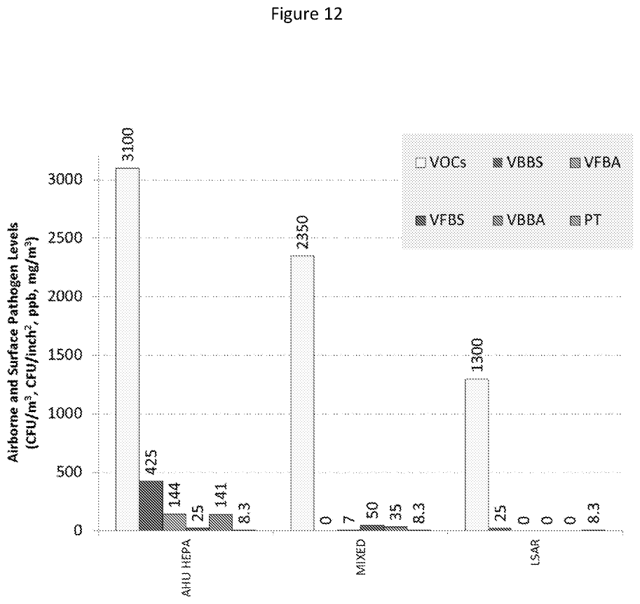

The overall study results are presented below in FIG. 12 and in Tables 5-8. All data were provided by independent third party laboratories after sampling the air and designated surfaces in each patient room associated with the specific study zone, as outlined in the methodology section. A comprehensive environmental assessment of viable bacterial, fungal and VOC pathogens was conducted each month and repeated a total of 4 times.

TABLE 5
Identification of viable bacteria and fungi by air and on surface
within the three study zones (CF-AHU, MIXED, and LSAR).

| Zone | VFBA | VFBS | VBBA | VBBS |
|---|---|---|---|---|
| AHU-HEPA | Aspergillus | Aspergillus | Microcossus | Micrococcus lylae |
| | | | luteus | |
| | Cladosporium | Cladosporium | Staphylococcus | Staphylococcus |
| | | (remote, return) | spp.: capitis, | spp.: capitis, |
| | | | epidermidis, | haemolyticus |
| | | | haemolyticus, | (remote, return), |
| | | | saprophyticus | hominis, |
| | | | | saprophyticus |
| | | | | (return) |
| | | | Gram negative rod | Gram negative rod |
| | | | | (return) |
| | | | Bacillus spp.: | Gram positive |
| | | | clausii, | cocci (remote, |
| | | | licheniformis | return) |
| | | | Dermobacter | |
| | | | hominis | |
| | | | Kocuria palustris | |
| MIXED | Yeast | Rhodotorula | Staphylococcus | Nil |
| | | (return) | haemolyticus | |
| | | | Bacillus | |
| | | | licheniformis | |
| | | | Dietzia cinnamea | |
| | | | Streptococcus | |
| | | | anginosus | |
| LSAR | Nil | Nil | Nil | Staphylococcus |
| | | | | spp.: capitis |
| | | | | (patient remote), |
| | | | | epidermidis |
| | | | | (faceplate) |
| | | | | Corynebacterium |
| | | | | (patient remote) |

TABLE 6
Pathogen Characteristics of Zone CF-AHU (the Control Zone)

| | | Location Within |
|---|---|---|
| Pathogen | Association with Patient Illness | Clinical Space |
| Aspergillus | Associated with pulmonary infections, | Circulating air |
| | infections to skin lesions | in patient room |
| | | Patient Remote |
| | | HVAC Return |
| Cladosporium | Associated with infections to skin, sinuses | Circulating air |
| | and lungs, significant allergens impacting | in patient room |
| | asthmatics and patients with respiratory | Patient Remote |
| | diseases, spores produce toxic VOCs | HVAC Return |
| Staphylococcus | Associated with urinary tract infections | Circulating air |
| saprophyticus | | in patient room |
| | | Return |
| Staphylococcus | Skin flora and low association with HAIs | Circulating air |
| epidermidis | | in patient room |
| Staphylococcus | Natural skin flora often associated with | Circulating air |
| capitis | infections caused by catheters and aortic | in patient room |
| | valves | Patient Remote |
| | | HVAC Return |
| Micrococcus | Source is typically patient-oriented, mouth, | Circulating air |
| luteus | mucosae, oropharynx and upper respiratory | in patient room |
| | tract, often associated with ill patients | |
| Staphylococcus | Antibiotic resistant and associated with | Circulating air |
| haemolyticus | skin flora | in patient room |
| | | Patient Remote |
| | | HVAC Return |
| Bacillus | Associated with respiratory infections and | Circulating air |
| clausii | GI disorders, produces antimicrobial | in patient room |
| | substances active against Staphylococcus | |
| | aureus, C. difficile, Enterococcus faecium | |
| Bacillus | Associated with soil and bird plumage | Circulating air |
| licheniformis | | in patient room |
| Dermabacter | Associated with wound infections, | Circulating air |
| hominis | abscesses, + blood cultures | in patient room |
| Kocuria | Pathogen responsible for UTIs | Circulating air |
| palustris | | in patient room |
| Gram + Cocci | Staphylococcus aureus and Streptococcus | Patient Remote |
| | pyogenes are two of the most common | HVAC Return |
| | causes of hospital-acquired pneumonia, | |
| | septicemia, folliculitis and surgical site | |
| | infections | |
| Gram | Associated with E. coli, Salmonella, | Circulating Air |
| negative rods | Shigella, Pseudomonas, severe GI illness | in Patient Room |
| | | HVAC Return |
| Micrococcus lylae | Associated with skin flora, opportunistic | HVAC Return |
| | pathogen in immunocompromised patients | |
| Staphylococcus | Associated with infections in | HVAC Return |
| hominis | immunocompromised patients | |

Finally, there were 3100 ppb VOCs and <8.3 mg/cubic meter of non-viable particulates in CF-AHU Zone.

TABLE 7
Pathogen Characteristics of MIXED Zone -
PARTIAL REMEDIATION (CF-AHU and 35% LSAR)

| | Association with | Location Within |
|---|---|---|
| Pathogen | Patient Illness | Clinical Space |
| Yeast | Associated with pulmonary | Circulating air |
| | infections, skin lesion | in patient room |
| | infections | |
| Rhodotorula | Common clinical contaminant | HVAC Return |
| | associated with soil and water | |
| Staphylococcus | Antibiotic resistant and | Circulating air |
| haemolyticus | associated with skin flora | in patient room |
| Bacillus | Associated with soil and bird | Circulating air |
| licheniformis | plumage | in patient room |
| Dietzia | Associated with catheter, | Circulating air |
| cinnamea | orthopaedic prosthesis- | in patient room |
| | associated infecgtions in | |
| | immunocompromised patients | |
| Streptococcus | Common cause of absesses, | Circulating air |
| anginosus | abdominal and thoracic | in patient room |
| | infections, endocarditis and | |
| | bacteremia | |

Finally, there were no viable bacteria by swab, 2350 ppb VOCs and <8.3 mg/cubic meter of non-viable particulates in the MIXED Zone.

TABLE 8
Pathogen Characteristics of LSAR Zone Patient Rooms

| | | Location Within |
|---|---|---|
| Pathogen | Association with Patient Illness | Clinical Space |
| Staphylococcus | Skin flora and low association | IV Control |
| epidermidis | with HAIs | Faceplate |
| Corynebacterium | Normal skin flora, low | Patient Remote |
| | association with infections, | |
| | prosthetic devices | |
| Staphylococcus | Natural skin flora often | Patient Remote |
| capitis | associated with infections caused | |
| | by catheters and aortic valves | |

Finally, there were no viable bacteria by air, no viable bacteria by swab, no viable fungi by air, 1300 ppb VOCs and <8.3 mg/cubic meter of non-viable particulates in air purification system of the disclosure Zone. The reduction in VOCs is due to the remediation of viable fungal spores and their concomitant production of fungal VOCs. As there was a HEPA filter in place on the serving air-handling unit, all air was HEPA-filtered. This was confirmed by the non-viable particulate assessment of <8.3 mg/cubic meter in all study zones.

### Discussion

Often neglected, indoor air quality is an important component of ensuring healthy and safe environment across various health-care facilities [Maroni, M., B. Seifert, and T. Lindvall, Indoor air quality: a comprehensive reference book. Vol. 3. 1995: Elsevier.]. It is well established that there exists “strong and sufficient evidence” of the association between ventilation, air movements in buildings, and the transmission of bacterial, fungal, and viral infectious diseases [Li, Y., et al., Role of ventilation in airborne transmission of infectious agents in the built environment—a multidisciplinary systematic review. Indoor air, 2007. 17(1): p. 2-18.1. Consequently, the need for high efficiency/reliabile air filtration becomes a necessity, especially in critical environments such as acute care wards, critical care units, isolation units, and operating rooms [Mahieu, L., et al., A prospective study on factors influencing aspergillus spore load in the air during renovation works in a neonatal intensive care unit. Journal of Hospital Infection, 2000. 45(3): p. 191-197; O'connell, N. and H. Humphreys, Intensive care unit design and environmental factors in the acquisition of infection. Journal of Hospital Infection, 2000. 45(4): p. 255-262; MacDonald, A., Protective air enclosures in health buildings: PAF White The Macmillan Press Ltd., 182 pp., ISBN 0 333 27678 7£ 15.00 (1981). 1981, Pergamon]. The current project highlights the importance of an integrated system, such as the advanced air purification system as disclosed herein, in the modern health-care environment. The subsequent discussion will synthesize our study's results in the context of acute care hospital setting.

Perhaps most importantly, there was a substantial decrease in air contaminants across all measurement categories. As the degree of air remediation increased from CF-AHU Zone or the control floor to comprehensive coverage in the patient rooms in LSAR Zone, a significant decrease in airborne bacterial, fungal and VOC load was observed. The decrease in both bacterial and fungal loads within the air was concomitant with a significant decrease observed on commonly touched clinical and patient surfaces. Within the control zone, many of the pathogens identified in air samples from patient rooms were also found on commonly touched patient surfaces and on the return vents of the room. These data provide a significant contribution to our understanding of the airflow and path of aerosolized pathogens within the typical clinical space.

Previously published data show a strong relationship between the presence of airborne fungal spores and air quality in the hospital setting [Araujo, R., J. P. Cabral, and A. G. Rodrigues, Air filtration systems and restrictive access conditions improve indoor air quality in clinical units: Penicillium as a general indicator of hospital indoor fungal levels. American journal of infection control, 2008. 36(2): p. 129-134]. Viable fungi species of Aspergillus and Cladosporium were speciated and quantitated within the control zone patient rooms. Our results demonstrate a substantial decrease in fungal spore detection rates when using LAS-APS technology, as compared to the other approaches.

The presence of bacteria, both in the air and on various surfaces, has been shown to be deleterious to health-care outcomes. In addition to the fungal species, viable bacterial species were also identified within the patient rooms of the control zone. Staphylococcus saprophyticus, Staphylococcus epidermidis, Staphylococcus capitis, Micrococcus luteus, Staphylococcus haemolyticus, Bacillus clausii, Bacillus licheniformis, Dermabacter hominis, Kocuria palustris, Gram+Cocci, Gram negative rods, Micrococcus lylae and Staphylococcus hominis were found in both the recirculating air of the patient room and on the patient remote and HVAC return. Many of the aerosolized pathogens found within the recirculating air were found on the HVAC return vents. Presence of these pathogens on the return vents confirms their aerosolized nature and threat to the clinical spaces also served by the recirculated air. With the exception of Bacillus licheniformis, each of these pathogens are associated with patient illness and infections. The sources of the above airborne pathogens are most likely the patients, visitors, and healthcare workers.

It is also important to note that patient rooms in the MIXED Zone received approximately 35% of their recirculated air from the rooms from the LSAR Zone and thus benefited from the installed LAS-APS filtration capacity. The zone also served as an “internal control” as it was located on same the floor as LSAR Zone. Viable yeast was found in the circulating air of the patient rooms in LSAR Zone and viable rhodotorula was found on the HVAC return vent. Although at a significantly reduced level from that observed in CF-AHU Zone, viable bacteria was identified within the air of the patient rooms of MIXED Zone. Staphylococcus haemolyticus, Dietzia cinnamea and Streptococcus anginosus, each a potential source of patient illness and infection, were identified in the patient rooms of MIXED Zone. Bacillus licheniformis was also identified but is not associated as a source of patient illness or infection. Interestingly, there were no viable bacteria found on the surfaces swabbed in MIXED Zone. VOCs were reduced over that assessed in CF-AHU Zone. The reduction of viable fungi in MIXED Zone corresponded to the simultaneous reduction in fungal VOC sources.

The patient rooms in LSAR Zone received all of their supply and recirculated air from the LAS-APS installation. There were no viable fungi by air or swab detected in the patient rooms in LSAR Zone. Likewise, there were no viable bacteria by air detected in the patient rooms in LSAR Zone. Low levels of Staphylococcus epidermidis were found on the IV Control Faceplate, and Corynebacterium and Staphylococcus capitis were found on the patient remote. Because no viable bacteria were identified within the air of the patient rooms in LSAR Zone, the surface bacteria identified on the patient remote and IV Control Faceplate were most likely due to direct surface-to-surface contact. The lowest levels of VOCs were found in the patient rooms of LSAR Zone as these rooms demonstrated no viable fungi in the circulating air.

The vast majority of infectious surface fomites originate from the air, and may be directed onto surfaces by air flow generated by in-room fans and air conditioning systems [Boone, S. A. and C. P. Gerba, Significance of fomites in the spread of respiratory and enteric viral disease. Applied and Environmental Microbiology, 2007. 73(6): p. 1687-1696; Lopez, G. U., et al., Transfer efficiency of bacteria and viruses from porous and nonporous fomites to fingers under different relative humidity. Applied and environmental microbiology, 2013: p. AEM. 01030-13; Greene, V., R. Bond, and G. Michaelsen, Air Purification in Hospitals. Cleaning and Purification of Air in Buildings, 1960 (797): p. 161. Consequently, a reduction in airborne bacterial and fungal pathogens should be associated with a reduction in surface fomites. Overall reduction of airborne and surface bacterial and fungal pathogens responsible for patient illness and infections should result in a reduction of associated illnesses, HAI rates, and improve metrics of patient care inclusive of, but not limited to, length of stay and readmission rates. Improvements in these outcome metrics should, by association, correlate to risk mitigation and cost avoidance.

It is important to note that this represents the first comprehensive evaluation of infectious and aerosolized pathogens and their speciation, location and concentration within a typical hospital setting. This provides important data regarding the complex relationship between airborne pathogens and air filtration methodologies in the context of the molecular and microbial epidemiology of illness and infections in the clinical setting. A greater understanding of the role of airborne pathogens in illness in the clinical setting will help facilitate the identification of proper and more optimal levels of remediation.

In the modern healthcare environment, organizations strive to provide optimal patient experience by improving the quality of patient care, enhancing clinical outcomes, while at the same time containing associated costs. Rarely is there an opportunity to utilize technology that positively impacts quality and cost of hospital care without a detrimental “trade off” or major changes in existing behaviors or protocols. The data clearly demonstrates a significant reduction across all forms of air contamination following the installation of the advanced air purification system as disclosed herein. These results represent an important milestone for this critical and often neglected area of health-care facility operations and maintenance.

While embodiments of the disclosure have been described in detail and with reference to specific examples thereof, it will be apparent to one skilled in the art that various changes and modifications can be made therein without departing from the spirit and scope thereof.

1. A method of reducing at least one of airborne biological contaminants, chemical contaminants, and particulate contaminants in a health care facility by providing purified air, the method comprising the steps of:
providing an air purifier in a health care facility;
providing source air to the air purifier;
purifying the source air with the air purifier, thereby providing purified air,
wherein the purified air has characteristics selected from the group consisting of:
a. a TVOC content of less than about 5 ppb;
b. a Biologicals content of less than about 1 CFU/M³;
c. a Particulate content of from about 1,000 0.3 micrometer particles per ft³ of air to about 30,000 0.3 micrometer particles per ft³ of air, or from about 600 0.5 micrometer particles per ft³ of air to about 10,000 0.5 micrometer particles per ft³ of air; and
d. combinations thereof,
supplying the purified air to the health care facility.

2. The method of claim 1, wherein the health care facility is a hospital.

3. The method of claim 1, wherein the health care facility is selected from the group consisting of a medical unit, a surgical unit, a critical care unit, an intensive care unit, an emergency care unit, a pediatric unit, an emergency unit, an outpatient unit, a specialty care unit, a dermatology unit, an endocrinology unit, a gastroenterology unit, an internal medicine unit, an oncology unit, a neurology unit, an orthopedic unit, an ophthalmic unit, an ear nose and throat unit, a neonatal unit, an obstetrics and gynecology unit, a cardiac unit, a psychiatric unit, a post-operative recovery unit, a radiology unit, a plastic surgery unit and an urology unit.

4. The method of claim 1, wherein the airborne biological contaminants are selected from the group consisting of viral contaminants, bacterial contaminants, fungal contaminants, parasitic contaminants, and combinations thereof.

5. The method of claim 1, to wherein the viral contaminants are selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, astrovirus, HAV, rotavirus, and combinations thereof.

6. The method of claim 1, wherein the bacterial contaminants are selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof.

7. The method of claim 1, wherein the fungal contaminants are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof.

8. The method of claim 1, wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof.

9. The method of claim 1, wherein the chemical contaminants are selected from the group consisting of tobacco smoke, engine exhaust, VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like; VOCs including organic solvents, certain paint additives, aerosol spray can propellants, fuels (such as gasoline, and kerosene), petroleum distillates, dry cleaning products, and many other industrial and consumer products ranging from office supplies to building materials; VOCs naturally emitted by a number of plants and trees, VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, methyl ethyl ketone, and combinations thereof.

10. The method of claim 1, wherein the particulate contaminants are selected from the group consisting of indoor allergens, dust mite feces, dander, cockroach-derived allergens, and combinations thereof.

11. The method of claim 1, wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier that is an in-room unit that is an in-ceiling unit that purifies in-room air, and an air purifier which is an in-room unit that is a ceiling unit that purifies in-room air.

12. A method of reducing healthcare acquired infections (HAIs) in a health care facility by providing purified air, the method comprising the steps of:
providing an air purifier in a health care facility;
providing source air to the air purifier;
purifying the source air with the air purifier, thereby providing purified air,
wherein the purified air has characteristics selected from the group consisting of:
a. a TVOC content of less than about 5 ppb;
b. a Biologicals content of less than about 1 CFU/M³;
c. a Particulate content of from about 1,000 0.3 micrometer particles per ft³ of air to about 30,000 0.3 micrometer particles per ft³ of air, or from about 600 0.5 micrometer particles per ft³ of air to about 10,000 0.5 micrometer particles per ft³ of air; and
d. combinations thereof,
supplying the purified air to the health care facility.

13. The method of claim 12, wherein the health care facility is a hospital.

14. The method of claim 12, wherein the health care facility is selected from the group consisting of a medical unit, a surgical unit, a critical care unit, an intensive care unit, an emergency care unit, a pediatric unit, an emergency unit, an outpatient unit, a specialty care unit, a dermatology unit, an endocrinology unit, a gastroenterology unit, an internal medicine unit, an oncology unit, a neurology unit, an orthopedic unit, an ophthalmic unit, an ear nose and throat unit, a neonatal unit, an obstetrics and gynecology unit, a cardiac unit, a psychiatric unit, a post-operative recovery unit, a radiology unit, a plastic surgery unit, and an urology unit.

15. The method of claim 12, wherein the purified air has reduced biological contaminants compared to source air, and wherein the biological contaminants are selected from the group consisting of viral contaminants, bacterial contaminants, fungal contaminants, parasitic contaminants, and combinations thereof.

16. The method of claim 12, wherein the purified air has reduced viral contaminants compared to source air, and wherein the viral contaminants are selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, astrovirus, HAV, rotavirus, and combinations thereof.

17. The method of claim 12, wherein the purified air has reduced bacterial contaminants compared to source air, and wherein the bacterial contaminants are selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof.

18. The method of claim 12, wherein the purified air has reduced fungal contaminants compared to source air, and wherein the fungal contaminants are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof.

19. The method of claim 12, wherein the purified air has reduced parasitic contaminants compared to source air, and wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof.

20. The method of claim 12, wherein the chemical contaminants are selected from the group consisting of tobacco smoke, engine exhaust, VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like; VOCs including organic solvents, certain paint additives, aerosol spray can propellants, fuels (such as gasoline, and kerosene), petroleum distillates, dry cleaning products, and many other industrial and consumer products ranging from office supplies to building materials; VOCs naturally emitted by a number of plants and trees, VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, methyl ethyl ketone, and combinations thereof.

21. The method of claim 12, wherein the purified air has reduced particulate contaminants compared to source air, and wherein the particulate contaminants are selected from the group consisting of indoor allergens, dust mite feces, dander, cockroach-derived allergens, and combinations thereof.

22. The method of claim 12, wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier that is an in-room unit that is an in-ceiling unit that purifies in-room air, and an air purifier which is an in-room unit that is a ceiling unit that purifies in-room air.

23. A method of reducing healthcare acquired infections (HAIs) in a long term care facility by providing purified air, the method comprising the steps of:
providing an air purifier in a health care facility;
providing source air to the air purifier;
purifying the source air with the air purifier, thereby providing purified air,
wherein the purified air has characteristics selected from the group consisting of:
a. a TVOC content of less than about 5 ppb;
b. a Biologicals content of less than about 1 CFU/M³;
c. a Particulate content of from about 1,000 0.3 micrometer particles per ft³ of air to about 30,000 0.3 micrometer particles per ft³ of air, or from about 600 0.5 micrometer particles per ft³ of air to about 10,000 0.5 micrometer particles per ft³ of air; and
d. combinations thereof,
supplying the purified air to the long term care facility.

24. The method of claim 23, wherein the long term care facility is selected from the group consisting of a Inpatient Hospice, Long Term Care Unit, Long Term Care Alzheimer's Unit, Long Term Care Behavioral Health/Psych Unit, Ventilator Dependent Unit, and a Long Term Care Rehabilitation Unit.

25. The method of claim 23, wherein the purified air has reduced biological contaminants compared to source air, and wherein the biological contaminants are selected from the group consisting of viral contaminants, bacterial contaminants, fungal contaminants, parasitic contaminants, and combinations thereof.

26. The method of claim 23, wherein the purified air has reduced viral contaminants compared to source air, and wherein the viral contaminant is selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, astrovirus, HAV, rotavirus, and combinations thereof.

27. The method of claim 23, wherein the purified air has reduced bacterial contaminants compared to source air, and wherein the bacterial contaminants are selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof.

28. The method of claim 23, wherein the purified air has reduced fungal contaminants compared to source air, and wherein the fungal contaminants are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof.

29. The method of claim 23, wherein the purified air has reduced parasitic contaminants compared to source air, and wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof.

30. The method of claim 23, wherein the chemical contaminants are selected from the group consisting of tobacco smoke, engine exhaust, VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like; VOCs including organic solvents, certain paint additives, aerosol spray can propellants, fuels (such as gasoline, and kerosene), petroleum distillates, dry cleaning products, and many other industrial and consumer products ranging from office supplies to building materials; VOCs naturally emitted by a number of plants and trees, VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, methyl ethyl ketone, and combinations thereof.

31. The method of claim 23, wherein the purified air has reduced particulate contaminants compared to source air, and wherein the particulate contaminants are selected from the group consisting of indoor allergens, dust mite feces, dander, cockroach-derived allergens, and combinations thereof.

32. The method of claim 23, wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier that is an in-room unit that is an in-ceiling unit that purifies in-room air, and an air purifier which is an in-room unit that is a ceiling unit that purifies in-room air.

33. A method of reducing at least one of airborne biological contaminants, chemical contaminants, and particulate contaminants in a long term care facility by providing purified air, the method comprising the steps of:
providing an air purifier in a long term care facility;
providing source air to the air purifier;
purifying the source air with the air purifier, thereby providing purified air,
wherein the purified air has characteristics selected from the group consisting of:
a. a TVOC content of less than about 5 ppb;
b. a Biologicals content of less than about 1 CFU/M³;
c. a Particulate content of from about 1,000 0.3 micrometer particles per ft³ of air to about 30,000 0.3 micrometer particles per ft³ of air, or from about 600 0.5 micrometer particles per ft³ of air to about 10,000 0.5 micrometer particles per ft³ of air; and
d. combinations thereof,
supplying the purified air to the long term care facility.

34. The method of claim 33, wherein the long term care facility is selected from the group consisting of a Inpatient Hospice, Long Term Care Unit, Long Term Care Alzheimer's Unit, Long Term Care Behavioral Health/Psych Unit, Ventilator Dependent Unit, and a Long Term Care Rehabilitation Unit.

35. The method of claim 33, wherein the airborne biological contaminants are selected from the group consisting of viral contaminants, bacterial contaminants, fungal contaminants, parasitic contaminants, and combinations thereof.

36. The method of claim 33, wherein the viral contaminants are selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, astrovirus, HAV, rotavirus, and combinations thereof.

37. The method of claim 33, wherein the bacterial contaminant is selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof.

38. The method of claim 33, wherein the fungal contaminant are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof.

39. The method of claim 33, wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof.

40. The method of claim 33, wherein the chemical contaminants are selected from the group consisting of tobacco smoke, engine exhaust, VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like; VOCs including organic solvents, certain paint additives, aerosol spray can propellants, fuels (such as gasoline, and kerosene), petroleum distillates, dry cleaning products, and many other industrial and consumer products ranging from office supplies to building materials; VOCs naturally emitted by a number of plants and trees, VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, methyl ethyl ketone, and combinations thereof.

41. The method of claim 33, wherein the particulate contaminants are selected from the group consisting of indoor allergens, dust mite feces, dander, cockroach-derived allergens, and combinations thereof.

42. The method of claim 33, wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier that is an in-room unit that is an in-ceiling unit that purifies in-room air, and an air purifier which is an in-room unit that is a ceiling unit that purifies in-room air.

43. A method of reducing at least one of airborne biological contaminants, chemical contaminants, and particulate contaminants in a laboratory facility by providing purified air, the method comprising the steps of:
providing an air purifier in a long term care facility;
providing source air to the air purifier;
purifying the source air with the air purifier, thereby providing purified air,
wherein the purified air has characteristics selected from the group consisting of:
a. a TVOC content of less than about 5 ppb;
b. a Biologicals content of less than about 1 CFU/M³;
c. a Particulate content of from about 1,000 0.3 micrometer particles per ft³ of air to about 30,000 0.3 micrometer particles per ft³ of air, or from about 600 0.5 micrometer particles per ft³ of air to about 10,000 0.5 micrometer particles per ft³ of air; and
d. combinations thereof,
supplying the purified air to the laboratory facility.

44. The method of claim 43, wherein the laboratory facility is selected from the group consisting of a laboratory facility for biochemistry, bioinformatics, biotechnology, cell biology, chemical biology, cell therapy, cell and organ transplantation, developmental biology, ecology, endocrinology, epidemiology, evolution, genetics, gene therapy, genomics, gerontology, immunology, infectious diseases, microbiology, molecular biology, nephrology, neurology, ophthalmology, pediatrics, pharmacology, physiology, plant biology, psychiatry, public health, structural biology, surgery, urology, drug discovery, molecular therapeutics, epidemiology, carcinogenesis, inflammation, pain, nutrition, reproduction, virology, toxicology, pathology, dermatology, gastroenterology, musculoskeletal studies, pregnancy, pulmonary studies, breast cancer, cardiovascular studies, cerebrospinal research, allergy and asthma studies, hepatology, atherosclerosis, diabetes studies, hematology, oncology, osteoporosis studies, rheumatology studies, vaccine studies, circadian rhythms studies, proteome studies, respiratory research, thrombosis studies, anti-viral and anti-microbial and anti-parasite studies gene regulation studies, cell culture studies of all kinds, organ culture and transplant studies of all kinds, protein production studies, in vitro and in vivo cell growth, in vitro and in vivo organ growth, and differentiation studies of all kinds.

45. The method of claim 43, wherein the airborne biological contaminants are selected from the group consisting of viral contaminants, bacterial contaminants, fungal contaminants, parasitic contaminants, and combinations thereof.

46. The method of claim 43, wherein the viral contaminant is selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, astrovirus, HAV, rotavirus, and combinations thereof.

47. The method of claim 43, wherein the bacterial contaminants are selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof.

48. The method of claim 43, wherein the fungal contaminants are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof.

49. The method of claim 43, wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof.

50. The method of claim 43, wherein the chemical contaminants are selected from the group consisting of Tobacco smoke, engine exhaust, VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like; VOCs including organic solvents, certain paint additives, aerosol spray can propellants, fuels (such as gasoline, and kerosene), petroleum distillates, dry cleaning products, and many other industrial and consumer products ranging from office supplies to building materials; VOCs naturally emitted by a number of plants and trees, VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, methyl ethyl ketone, and combinations thereof.

51. The method of claim 43, wherein the particulate contaminants are selected from the group consisting of indoor allergens, dust mite feces, dander, cockroach-derived allergens, and combinations thereof.

52. The method of claim 43, wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier which is an in-room unit that is an in-ceiling unit that purifies in-room air, and an air purifier which is an in-room unit that is a ceiling unit that purifies in-room air.

53. A method of providing purified air to a laboratory, the method comprising the steps of:
providing an air purifier in a laboratory facility;
providing source air to the air purifier;
purifying the source air with the air purifier, thereby providing purified air,
wherein the purified air has characteristics selected from the group consisting of:
a. a TVOC content of less than about 5 ppb;
b. a Biologicals content of less than about 1 CFU/M³;
c. a Particulate content of from about 1,000 0.3 micrometer particles per ft³ of air to about 30,000 0.3 micrometer particles per ft³ of air, or from about 600 0.5 micrometer particles per ft³ of air to about 10,000 0.5 micrometer particles per ft³ of air; and
d. combinations thereof,
supplying the purified air to the laboratory facility.

54. The method of claim 53, wherein the laboratory facility is selected from the group consisting of a laboratory facility for biochemistry, bioinformatics, biotechnology, cell biology, chemical biology, cell therapy, cell and organ transplantation, developmental biology, ecology, endocrinology, epidemiology, evolution, genetics, gene therapy, genomics, gerontology, immunology, infectious diseases, microbiology, molecular biology, nephrology, neurology, ophthalmology, pediatrics, pharmacology, physiology, plant biology, psychiatry, public health, structural biology, surgery, urology, drug discovery, molecular therapeutics, epidemiology, carcinogenesis, inflammation, pain, nutrition, reproduction, virology, toxicology, pathology, dermatology, gastroenterology, musculoskeletal studies, pregnancy, pulmonary studies, breast cancer, cardiovascular studies, cerebrospinal research, allergy and asthma studies, hepatology, atherosclerosis, diabetes studies, hematology, oncology, osteoporosis studies, rheumatology studies, vaccine studies, circadian rhythms studies, proteome studies, respiratory research, thrombosis studies, anti-viral and anti-microbial and anti-parasite studies gene regulation studies, cell culture studies of all kinds, organ culture and transplant studies of all kinds, protein production studies, in vitro and in vivo cell growth, in vitro and in vivo organ growth, and differentiation studies of all kinds.

55. The method of claim 53, wherein the purified air has reduced biological contaminants compared to source air, wherein the biological contaminants are selected from the group consisting of viral contaminants, bacterial contaminants, fungal contaminants, parasitic contaminants, and combinations thereof.

56. The method of claim 53, wherein the purified air has reduced viral contaminants compared to source air, wherein the viral contaminants are selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, astrovirus, HAV, rotavirus, and combinations thereof.

57. The method of claim 53, wherein the purified air has reduced bacterial contaminants compared to source air, wherein the bacterial contaminants are selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof.

58. The method of claim 53, wherein the purified air has reduced fungal contaminants compared to source air, wherein the fungal contaminants are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof.

59. The method of claim 53, wherein the purified air has reduced parasitic contaminants compared to source air, wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof.

60. The method of claim 53, wherein the chemical contaminants are selected from the group consisting of tobacco smoke, engine exhaust, VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like; VOCs including organic solvents, certain paint additives, aerosol spray can propellants, fuels (such as gasoline, and kerosene), petroleum distillates, dry cleaning products, and many other industrial and consumer products ranging from office supplies to building materials; VOCs naturally emitted by a number of plants and trees, VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, methyl ethyl ketone, and combinations thereof.

61. The method of claim 53, wherein the purified air has reduced particulate contaminants compared to source air, wherein the particulate contaminants are selected from the group consisting of indoor allergens, dust mite feces, dander, cockroach-derived allergens, and combinations thereof.

62. The method of claim 53, wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier which is an in-room unit that is an in-ceiling unit that purifies in-room air, and an air purifier which is an in-room unit that is a ceiling unit that purifies in-room air.

63. A method of performing a laboratory process in purified air, the method comprising the steps of:
providing an air purifier in a laboratory facility;
providing source air to the air purifier;
purifying the source air with the air purifier, thereby providing purified air,
wherein the purified air has characteristics selected from the group consisting of:
a. a TVOC content of less than about 5 ppb;
b. a Biologicals content of less than about 1 CFU/M³;
c. a Particulate content of from about 1,000 0.3 micrometer particles per ft³ of air to about 30,000 0.3 micrometer particles per ft³ of air, or from about 600 0.5 micrometer particles per ft³ of air to about 10,000 0.5 micrometer particles per ft³ of air; and
d. combinations thereof,
supplying the purified air to the laboratory facility, and performing the laboratory process.

64. The method of claim 63, wherein the laboratory process is selected from the group consisting of cell culture, gene therapy, viral production, biochemistry, bioinformatics, biotechnology, cell biology, chemical biology, cell therapy, cell and organ transplantation, developmental biology, ecology, endocrinology, epidemiology, evolution, genetics, gene therapy, genomics, gerontology, immunology, infectious diseases, microbiology, molecular biology, nephrology, neurology, ophthalmology, pediatrics, pharmacology, physiology, plant biology, psychiatry, public health, structural biology, surgery, urology, drug discovery, molecular therapeutics, epidemiology, carcinogenesis, inflammation, pain, nutrition, reproduction, virology, toxicology, pathology, dermatology, gastroenterology, musculoskeletal studies, pregnancy, pulmonary studies, breast cancer, cardiovascular studies, cerebrospinal research, allergy and asthma studies, hepatology, atherosclerosis, diabetes studies, hematology, oncology, osteoporosis studies, rheumatology studies, vaccine studies, circadian rhythms studies, proteome studies, respiratory research, thrombosis studies, anti-viral and anti-microbial and anti-parasite studies gene regulation studies, cell culture studies of all kinds, organ culture and transplant studies of all kinds, protein production studies, in vitro and in vivo cell growth, in vitro and in vivo organ growth, and differentiation studies of all kinds.

65. The method of claim 63, wherein the purified air has reduced biological contaminants compared to source air, wherein the biological contaminants are selected from the group consisting of viral contaminants, bacterial contaminants, fungal contaminants, parasitic contaminants, and combinations thereof.

66. The method of claim 63, wherein the purified air has reduced viral contaminants compared to source air, wherein the viral contaminants are selected from the group consisting of Influenza, Parvovirus, Echovirus, Coxsachie virus, Norwalk virus, Reovirus, Adenovirus, influenza A virus, Avian Influenza virus, Coronavirus (SARS), Newcastle disease, Haemophilus influenza, Vaccinia virus, Measles virus, Zika virus, Rhinovirus, Norovirus, Respiratory Syncytial Virus, Adenovirus, HPV, astrovirus, HAV, rotavirus, and combinations thereof.

67. The method of claim 63, wherein the purified air has reduced bacterial contaminants compared to source air, wherein the bacterial contaminants are selected from the group consisting of Anthrax, MRSA, Clostridium difficile spore, Pseudomonas aeruginosa, Legionella pneumophilia, Aspergillus, Tuberculosis, Vancomycin-resistant enterococcus, Acinetobacter, Klebsiella, Staphylococcus pneumonia, Streptococcus pneumonia, Mycobacterium tuberculosis, Staphylococcus, Streptococcus, Pseudomonas aeruginosa, Burkholderia cenocepacia, Mycobacterium avium, Chlamydophila, Ehrlichia, Rickettsia, Mycobacterium, Brucella, Francisella, Legionella, Listeria, and combinations thereof.

68. The method of claim 63, wherein the purified air has reduced fungal contaminants compared to source air, wherein the fungal contaminants are selected from the group consisting of Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum, and combinations thereof.

69. The method of claim 63, wherein the purified air has reduced parasitic contaminants compared to source air, wherein the parasitic contaminants are selected from the group consisting of Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, and combinations thereof.

70. The method of claim 63, wherein the chemical contaminants are selected from the group consisting of tobacco smoke, engine exhaust, VOCs from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like; VOCs including organic solvents, certain paint additives, aerosol spray can propellants, fuels (such as gasoline, and kerosene), petroleum distillates, dry cleaning products, and many other industrial and consumer products ranging from office supplies to building materials; VOCs naturally emitted by a number of plants and trees, VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, methyl ethyl ketone, and combinations thereof.

71. The method of claim 63, wherein the purified air has reduced particulate contaminants compared to source air, wherein the particulate contaminants are selected from the group consisting of indoor allergens, dust mite feces, dander, cockroach-derived allergens, and combinations thereof.

72. The method of claim 63, wherein the air purifier is selected from the group consisting of a unit installed into an existing HVAC system or AHU, an air purifier which is a stand-alone unit that is not part of an HVAC system or AHU, an air purifier which is a stand-alone unit which purifies in-room air, an air purifier which is an in-room unit that purifies in-room air, an air purifier which is an in-room unit that is an in-ceiling unit that purifies in-room air, and an air purifier which is an in-room unit that is a ceiling unit that purifies in-room air.
